SEQUENCE LISTING (56 sequences)

SEQ ID NO: 1 - protein, 291 aa, Rattus norvegicus
  REGION 1..291: note = Arylsulfotransferase IV
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 2 - protein, 295 aa, Homo sapiens
  REGION 1..295: note = Arylsulfotransferase
MELIQDTSRPPLEYVKGVPLIKYFAEALGPLQSFQARPDDLLISTYPKSGTTWVSQILDMIYQGGDLEKCHRAPIFMRVPFLEFKAPGIPSGMETLKDTPAPRLLKTHLPLALLPQTLLDQKVKVVYVARNAKDVAVSYYHFYHMAKVHPEPGTWDSFLEKFMVGEVSYGSWYQHVQEWWELSRTHPVLYLFYEDMKENPKREIQKILEFVGRSLPEETVDFVVQHTSFKEMKKNPMTNYTTVPQEFMDHSISPFMRKGMAGDWKTTFTVAQNERFDADYAEKMAGCSLSFRSEL

SEQ ID NO: 3 - protein, 294 aa, Gallus gallus
  REGION 1..294: note = Arylsulfotransferase
MGNDEVIRQDLGCLYDIPLYKCFVAGWPQVEAFQARPDDLLIATYPKSGTTWLSEILDAIYHDGDLEKCRRDAIYNRVPFLEMKAPGILSGVEQLEKIPSPRLVKTHLPVHLLPASFQEKDCKVIYMARNAKDVVISYYYFYQMAKIHPDPGTLSEFLQAFMDGKVAYGSWYKHVKGWWEKRHEKRLLYLFYEDMKKDPRREIQKILQFLGKEVAEETVARILHHTSFQEMKKNPATNYETMPTELMDHSLSPFMRKGISGDWANHFTVAQNERFDQHYQQQMAGSDLCFQMEA

SEQ ID NO: 4 - protein, 295 aa, Bos taurus
  REGION 1..295: note = Arylsulfotransferase
MELIQDTSRPPAKYVKGIPLIKYFAEALGPLESFEAWPDDLLISTYPKSGTTWVSEILDLIYQEGDLEKCQRAPVFLRVPFLEFSAPGVPTGVELLKDTPAPRLLKTHLPLALLPKTLLDQKVKVIYIARNAKDVAVSYYHFYRMAKVHPDPGTWDSFLEKFMAGEVCYGSWYQHVQEWWELSHTHPVLYLFYEDIKEDPKREIQKILEFIGRSLPEETVDHIVQRTSFKEMKKNPMTNYSTIPTAVMDHSISAFMRKGITGDWKSTFTVAQNELFEAHYAKKMAGCKLRFRWEL

SEQ ID NO: 5 - protein, 291 aa, synthetic construct
  REGION 1..291: note = Arylsulfotransferase IV Var01: I17F
MEFSRPPLVHVKGIPLFKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 6 - protein, 291 aa, synthetic construct
  REGION 1..291: note = Arylsulfotransferase IV Var04: F20L
MEFSRPPLVHVKGIPLIKYLAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 7 - protein, 291 aa, synthetic construct
  REGION 1..291: note = Arylsulfotransferase IV Var03: F20I
MEFSRPPLVHVKGIPLIKYIAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 8 - protein, 291 aa, synthetic construct
  REGION 1..291: note = Arylsulfotransferase IV Var05: F138H
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNHYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 9 - protein, 291 aa, synthetic construct
  REGION 1..291: note = Arylsulfotransferase IV Var06: Y236F
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNFTTIPTEIMDHNVSPFMRKGTTGDWKNTFTVAQNERFDAHYAKTMTDCDFKFRCEL

SEQ ID NO: 10 - protein, 291 aa, synthetic construct
  REGION 1..291: note = Arylsulfotransferase IV Var07: M244N
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEINDHNVSPFMRKGTTGDWKNTFTVAQNERFDAHYAKTMTDCDFKFRCEL

SEQ ID NO: 11 - protein, 291 aa, synthetic construct
  REGION 1..291: note = Arylsulfotransferase IV Var02: I17Y
MEFSRPPLVHVKGIPLYKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 12 - protein, 291 aa, synthetic construct
  REGION 1..291: note = Arylsulfotransferase IV Var08: I239D
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTDPTEIMDHNVSPFMRKGTTGDWKNTFTVAQNERFDAHYAKTMTDCDFKFRCEL

SEQ ID NO: 13 - protein, 291 aa, synthetic construct
  REGION 1..291: note = Arylsulfotransferase IV Var09:P6Q-P7D-L8A-V9G-V11L-W33R-K62D-A97S-N195D-T263H
MEFSRQDAGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 14 - protein, 291 aa, synthetic construct
  REGION 1..291: note = Arylsulfotransferase IV Var09-01: P6Q
MEFSRQPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 15 - protein, 291 aa, synthetic construct
  REGION 1..291: note = Arylsulfotransferase IV Var09-02: P7D
MEFSRPDLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 16 - protein, 291 aa, synthetic construct
  REGION 1..291: note = Arylsulfotransferase IV Var09-03: L8A
MEFSRPPAVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 17 - protein, 291 aa, synthetic construct
  REGION 1..291: note = Arylsulfotransferase IV Var09-04: V9G
MEFSRPPLGHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 18 - protein, 291 aa, synthetic construct
  REGION 1..291: note = Arylsulfotransferase IV Var09-05: V11L
MEFSRPPLVHLKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 19 - protein, 291 aa, synthetic construct
  REGION 1..291: note = Arylsulfotransferase IV Var09-06: W33R
MEFSRPPLVHVKGIPLIKYFAETIGPLQNFTARPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 20 - protein, 291 aa, synthetic construct
  REGION 1..291: note = Arylsulfotransferase IV Var09-07: K62D
MEFSRPPLVHVKGIPLIKYFAETIGPLQNFTAWPDDLLISTYPKSGTTWMSEILDMIYQGGDLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 21 - protein, 291 aa, synthetic construct
  REGION 1..291: note = Arylsulfotransferase IV Var09-08: A97S
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 22 - protein, 291 aa, synthetic construct
  REGION 1..291: note = Arylsulfotransferase IV Var09-09: N195D
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 23 - protein, 291 aa, synthetic construct
  REGION 1..291: note = Arylsulfotransferase IV Var09-10: T263H
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 24 - protein, 291 aa, synthetic construct
  REGION 1..291: note = Arylsulfotransferase IV Var09 less P6Q
MEFSRPDAGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 25 - protein, 291 aa, synthetic construct
  REGION 1..291: note = Arylsulfotransferase IV Var09 less P7D
MEFSRQPAGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 26 - protein, 291 aa, synthetic construct
  REGION 1..291: note = Arylsulfotransferase IV Var09 less L8A
MEFSRQDLGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 27 - protein, 291 aa, synthetic construct
  REGION 1..291: note = Arylsulfotransferase IV Var09 less V9G
MEFSRQDAVHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 28 - protein, 291 aa, synthetic construct
  REGION 1..291: note = Arylsulfotransferase IV Var09 less V11L
MEFSRQDAGHVKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 29 - protein, 291 aa, synthetic construct
  REGION 1..291: note = Arylsulfotransferase IV Var09 less W33R
MEFSRQDAGHLKGIPLIKYFAETIGPLQNFTAWPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 30 - protein, 291 aa, synthetic construct
  REGION 1..291: note = Arylsulfotransferase IV Var09 less K62D
MEFSRQDAGHLKGIPLIKYFAETIGPLQNFTARPDDLLISTYPKSGTTWMSEILDMIYQGGKLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 31 - protein, 291 aa, synthetic construct
  REGION 1..291: note = Arylsulfotransferase IV Var09 less A97S
MEFSRQDAGHLKGIPLIKYFAETIGPLQNFTARPDDLLISTYPKSGTTWMSEILDMIYQGGDLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 32 - protein, 291 aa, synthetic construct
  REGION 1..291: note = Arylsulfotransferase IV Var09 less N195D
MEFSRQDAGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 33 - protein, 291 aa, synthetic construct
  REGION 1..291: note = Arylsulfotransferase IV Var09 less T263H
MEFSRQDAGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 34 - protein, 291 aa, synthetic construct
  REGION 1..291: note = Arylsulfotransferase IV Var09 less K62D and less T263H
MEFSRQDAGHLKGIPLIKYFAETIGPLQNFTARPDDLLISTYPKSGTTWMSEILDMIYQGGKLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 35 - protein, 291 aa, synthetic construct
  REGION 1..291: note = Arylsulfotransferase IV Var09 less K62D, less N195D, and lessT263H
MEFSRQDAGHLKGIPLIKYFAETIGPLQNFTARPDDLLISTYPKSGTTWMSEILDMIYQGGKLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 36 - protein, 291 aa, synthetic construct
  REGION 1..291: note = Arylsulfotransferase IV Var09 plus I17F
MEFSRQDAGHLKGIPLFKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 37 - protein, 291 aa, synthetic construct
  REGION 1..291: note = Arylsulfotransferase IV Var09 plus I17Y
MEFSRQDAGHLKGIPLYKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 38 - protein, 291 aa, synthetic construct
  REGION 1..291: note = Arylsulfotransferase IV Var09 plus F20I
MEFSRQDAGHLKGIPLIKYIAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 39 - protein, 291 aa, synthetic construct
  REGION 1..291: note = Arylsulfotransferase IV Var09 plus F20L
MEFSRQDAGHLKGIPLIKYLAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 40 - protein, 291 aa, synthetic construct
  REGION 1..291: note = Arylsulfotransferase IV Var09 plus F138H
MEFSRQDAGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNHYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 41 - protein, 291 aa, synthetic construct
  REGION 1..291: note = Arylsulfotransferase IV Var09 plus Y236F
MEFSRQDAGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNFTTIPTEIMDHNVSPFMRKGTTGDWKNHFTVAQNERFDAHYAKTMTDCDFKFRCEL

SEQ ID NO: 42 - protein, 291 aa, synthetic construct
  REGION 1..291: note = Arylsulfotransferase IV Var09 plus I239D
MEFSRQDAGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTDPTEIMDHNVSPFMRKGTTGDWKNHFTVAQNERFDAHYAKTMTDCDFKFRCEL

SEQ ID NO: 43 - protein, 291 aa, synthetic construct
  REGION 1..291: note = Arylsulfotransferase IV Var09 plus M244N
MEFSRQDAGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEINDHNVSPFMRKGTTGDWKNHFTVAQNERFDAHYAKTMTDCDFKFRCEL

SEQ ID NO: 44 - protein, 291 aa, synthetic construct
  REGION 1..291: note = Arylsulfotransferase IV Var5A
MEFSRQDAGHLKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 45 - protein, 291 aa, synthetic construct
  REGION 1..291: note = Arylsulfotransferase IV Var5B
MEFSRPPLVHVKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 46 - protein, 291 aa, synthetic construct
  REGION 1..291: note = Arylsulfotransferase IV VarA5 plus W33R
MEFSRQDAGHLKGIPLIKYFAETIGPLQNFTARPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 47 - protein, 291 aa, synthetic construct
  REGION 1..291: note = Arylsulfotransferase IV Var5A plus K62D
MEFSRQDAGHLKGIPLIKYFAETIGPLQNFTAWPDDLLISTYPKSGTTWMSEILDMIYQGGDLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 48 - protein, 291 aa, synthetic construct
  REGION 1..291: note = Arylsulfotransferase IV Var5A plus A97S
MEFSRQDAGHLKGIPLIKYFAETIGPLQNFTAWPDDLLISTYPKSGTTWMSEILDMIYQGGKLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 49 - protein, 291 aa, synthetic construct
  REGION 1..291: note = Arylsulfotransferase IV Var5A plus N195D
MEFSRQDAGHLKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 50 - protein, 291 aa, synthetic construct
  REGION 1..291: note = Arylsulfotransferase IV Var5A plus T263H
MEFSRQDAGHLKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 51 - protein, 291 aa, synthetic construct
  REGION 1..291: note = Arylsulfotransferase IV Var5B plus P6Q
MEFSRQPLVHVKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 52 - protein, 291 aa, synthetic construct
  REGION 1..291: note = Arylsulfotransferase IV Var5B plus P7D
MEFSRPDLVHVKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 53 - protein, 291 aa, synthetic construct
  REGION 1..291: note = Arylsulfotransferase IV Var5B plus L8A
MEFSRPPAVHVKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 54 - protein, 291 aa, synthetic construct
  REGION 1..291: note = Arylsulfotransferase IV Var5B plus V9G
MEFSRPPLGHVKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 55 - protein, 291 aa, synthetic construct
  REGION 1..291: note = Arylsulfotransferase IV Var5B plus V11L
MEFSRPPLVHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKEDPKREIKKILEFLGRSLPEETVDSIVHHTSFKKMKENCMTNYTTIPTEIMDHNVSPFMRKGTTGDWKNHFTVAQNERFDAHYAKTMTDCDFKFRCEL

SEQ ID NO: 56 - protein, 291 aa, synthetic construct
  REGION 1..291: note = Arylsulfotransferase IV V09 less N195D plus Y236F
MEFSRQDAGHLKGIPLIKYFAETIGPLQNFTARPDDLLISTYPKSGTTWMSEILDMIYQGGDLEKCGRAPIYARVPFLEFKCPGVPSGLETLEETPSPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNFTTIPTEIMDHNVSPFMRKGTTGDWKNHFTVAQNERFDAHYAKTMTDCDFKFRCEL

(19) United States
(12) Patent Application Publication
(10) Pub. No.: US 2023/0130811 A1
(43) Pub. Date: Apr. 27, 2023

(54) USES AND METHODS FOR SULFATING A SUBSTRATE WITH A MUTATED ARYLSULFOTRANSFERASE

(71) Applicant: SANOFI, Paris (FR)

(72) Inventors: Aymeric DEPLACE, Paris (FR); Emanuele MONZA, Barcelona (ES); Davide PANIGADA, Paris (FR); Anke STEINMETZ, Paris (FR)

(21) Appl. No.: 17/936,598

(22) Filed: Sep. 29, 2022

(30) Foreign Application Priority Data
Sep. 30, 2021 (EP) 21306358.9

(51) Int. Cl. C12P 19/04; C12N 9/10; C12P 11/00

(52) U.S. Cl. CPC C12P 19/04; C12N 9/13; C12Y 208/02022; C12P 11/00

(57) ABSTRACT

The invention relates to uses and methods implementing a non-naturally occurring mutated arylsulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, and (ii) an amino acid sequence having at least 60% sequence identity with amino acids sequence SEQ ID NO: 1 for sulfating a substrate. The mutated arylsulfotransferase may have a sulfotransferase activity for converting adenosine 3′,5′-bisphosphate (PAP) into 3′-phosphoadenosine-5′-phosphosulfate (PAPS) enhanced compared to the wild-type enzyme.

### RELATED APPLICATIONS

This application claims priority to European Patent Application No. 21306358.9, filed Sep. 30, 2021, the entire disclosure of which is hereby incorporated herein by reference.

### SEQUENCE LISTING SUBMITTED ELECTRONICALLY

The instant application contains a Sequence Listing which has been submitted electronically in XML format and is hereby incorporated by reference in its entirety. Said XML copy, created on Sep. 20, 2022, is named 733806_SA9-346_ST26.xml and is 87,525 bytes in size.

### TECHNICAL FIELD

The invention relates to mutant enzymes with enhanced properties. The invention further relates to mutated, or non-naturally occurring, arylsulfotransferase with enhanced sulfation activity. Also, the invention relates to methods for sulfation of substrates using these mutants. Methods and systems for synthesizing heparin compounds are also provided.

### TECHNICAL BACKGROUND

Sulfation is a conjugation process involved in numerous biological processes including synthesis of proteins, peptides or glycosaminoglycans (GAGs), detoxification, hormone regulation, molecular recognition, cell signaling, or viral entry into cells.

The sulfation reaction needs a sulfotransferase (SULT) enzyme as a catalyst and a co-substrate as a sulfuryl (or sulfo group) donor. A universal donor for these reactions is 3′-phosphoadenosine 5′-phosphosulfate (PAPS). Sulfotransferases (SULTS) are a family of enzymes that transfer the sulfate group from PAPS onto usually a hydroxyl group of a target substrate.

Among the sulfonated glycosaminoglycans (GAGs) issued from sulfation processes, are heparan sulfate (HS) and heparin. Those GAGs are closely related highly sulfated polysaccharides consisting of repeating disaccharide units of glucuronic acid or iduronic acid linked to glucosamine and involved in a number of important biological and pharmacological activities.

HS is a component of the cell surface and extracellular matrix and is involved in a wide range of physiologic and pathophysiologic functions, such as blood coagulation and viral infection (Esko and Selleck (2002) Annu. Rev. Biochem. 71, 435-471; Liu and Thorp (2002) Med. Res. Rev. 22, 1-25). It is a highly charged polysaccharide comprising 1→4-linked glucosamine and glucuronic/iduronic acid units that contain both N- and O-sulfo groups.

Heparin, a specialized form of heparan sulfate, is found primarily intracellulariy in the granules of mast cells and is a commonly used anticoagulant drug. Three forms of heparin can be found on the market: unfractionated (UF) heparin (MW_{avg} ˜14000 Da); a low molecular weight heparin (MW_{avg}˜6000 Da); and the synthetic ULMW heparin pentasaccharide (MW 1508.3 Da). UF heparin is used in surgery and kidney dialysis due to its relatively short half-life while LMW heparins and the ULMW heparin are intended for preventing venous thrombosis among high-risk patients.

In the body, HS and heparin are biosynthesized in the endoplasmic reticulum (ER) and the Golgi compartments. Glycosyltransferase enzymes catalyze the alternating addition of UDP-activated β-D-glucuronic acid (GlcA) and N-acetylglucosamine (GlcNAc) residues to generate a polysaccharide chain, which is then modified by N-deacetylase, C5-epimerase and sulfotransferase enzymes. N-deacetylase/N-sulfotransferases (NDST) replace N-acetyl groups with an N-sulfo group, and C5-epimerase and O-sulfotransferases (OSTs) work together to convert GlcA into α-L-iduronic acid (IdoA), and then into IdoA2S (addition of a 2-O-sulfo group). D-glucosamine residues are then modified by 6-O-sulfotransferases (6OSTs), followed by 3-O-sulfotransferases (3OSTs). Tissue specific expression of different enzyme isoforms fine-tunes the synthesis of HP and HS to produce different structures, allowing adaptation of function to the local cellular environment (Fu et al., Adv Drug Deliv Rev. 2016; 97:237-249).

Application of HS biosynthetic enzymes for generating large heparin and HS oligosaccharides with desired biological activities is now possible with the successful expression of recombinant heparin biosynthetic enzymes (Fu et al., Adv Drug Deliv Rev. 2016:97:237-249).

In the bioprocesses developed for synthesizing HS and heparin, OSTs act on N-sulfoheparosan in the presence of the cofactor 3′-phosphoadenosine-5′-phosphosulfate (PAPS) (Fu et al., Adv Drug Deliv Rev. 2016; 97:237-249). 3′-Phosphoadenosine-5′-phosphosulfate (PAPS) is a derivative of adenosine monophosphate that is phosphorylated at the 3′ position and has a sulfate group attached to the 5′ phosphate. It is the most common coenzyme involved in sulfotransferase reactions.

A cofactor recycling system, involving arylsulfotransferase-IV (AST-IV), may be used to convert, expensive cofactor 3′-phosphoadenosine-5′-phosphate (PAP) to PAPS by transferring a sulfo group from an inexpensive sacrificial donor, p-nitrophenyl sulfate (pNPS), to PAP, regenerating PAPS (Burkart et al., J Org Chem. 2000; 65(18):5565-5574; Xiong et al., J Biotechnol. 2013; 167(3):241-247). Such system has been used to produced heparan sulfate (Chen et al., J Biol Chem. 2005; 280(52):42817-42825) and heparin (WO 2010/040973). The reaction also produces p-nitrophenol (PNP) that can be recovered and chemically sulfonated. This cofactor regeneration system saves on cost, since PAPS is nearly 1000-fold more expensive than pNPS (Fu et al., Adv Drug Deliv Rev. 2016; 97:237-249).

PAPS, a universal sulfate donor and source of sulfate for all sulfotransferases, is a highly expensive and unstable molecule that has been an obstacle to the large-scale production of enzymatically sulfated products.

Therefore, there is a need to optimize the yield of the conversion, or recycling, of PAP into PAPS.

Introducing mutations in amino acids sequences of enzymes is known to affect negatively or positively the catalytic activity of the enzyme.

Guo et al. (Chem Biol Interact. 1994; 92(1-3):25-31) and Sheng et al. (Drug Metab Dispos. 2004; 32(5):559-565) describe mutated phenol sulfotransferases IV in which mutations induced a change of its relative specific activities or stereospecificity with respect to different substrate.

Marshall et al. (J Biol Chem. 1997; 272(14):9153-9160) and Lin et al. (Biochem Pharmacol. 2012; 84(2):224-23) disclose rat phenol sulfotransferase (rSULT1A1) mutants with various redox regulation capacities.

Berger et al. (PLoS One. 2011; 6(11):e26794) and Zhou et al. (3 Biotech. 2019; 9(6):246) describe human aryl sulfotransferase SULTA1 mutants with enhanced catalytic activity.

Sulfotransferase enzyme activity may be measured with various assays known in the art (Paul et al., Anal Bioanal Chem. 2012; 403(6):1491-1500).

There is a need to have enzyme usable in bioprocess to convert PAP into PAPS.

There is a need to have enzyme with an enhanced catalytic activity to convert PAP into PAPS.

There is a need to have arylsulfotransferase, such as rat arylsulfotransferase IV, with enhanced catalytic activity to convert PAP into PAPS.

There is a need to have arylsulfotransferase, such as rat arylsulfotransferase IV, with enhanced thermal stability.

There is a need to have methods for sulfation of substrate with lower cost and/or improved yield.

There is a need to have methods for sulfation of a N-sulfated heparosan, heparan sulfate or heparosan sulfate with lower cost and/or improved yield.

There is a need to have a method for biosynthesis heparin with lower cost and/or improved yield.

There is a need to have a method for biosynthesis heparin which can use a recycling system to convert 3′-phosphoadenosine-5′-phosphate (PAP) to 3′-phosphoadenosine-5′-phosphosulfate (PAPS).

The present has for purpose to satisfy all or part of these needs.

### SUMMARY

According to one of its objects, the present invention relates to a non-naturally occurring mutated arylsulfotransferase comprising (i) an amino acid substitution in at least one amino acid position selected among positions 6, 7, 8, 9, 11, 17, 20, 33, 62, 97, 138, 195, 236, 239, 244, 263 and combinations thereof, wherein the position is relative to the amino acids sequence of rat arylsulfotransferase IV SEQ ID NO: 1, and (II) an amino acid sequence having at least 60% sequence identity with amino acids sequence SEQ ID NO: 1, with the proviso that when said arylsulfotransferase is the rat arylsulfotransferase IV, the mutations are not F138A and/or Y236A.

As shown in the Examples illustrating the present disclosure, the inventors have surprisingly obtained a series of mutated arylsulfotransferases, in which some amino acids have been substituted, with enhanced catalytic activity to convert 3′,5′-adenosine-phosphate (PAP) into 3′-phosphoadenosine-5′-phosphosulfate (PAPS).

The mutated arylsulfotransferases as disclosed herein have a PAP→PAPS converting activity enhanced from at least 1.3-folds to up to 7-folds greater than the corresponding activity of the wild-type rat arylsulfotransferase.

The mutated arylsulfotransferases as disclosed herein can be advantageously used in sulfation bioprocess systems.

The mutated arylsulfotransferases as disclosed herein can be advantageously used in a recycling system of sulfation bioprocess systems for enhancing the conversion activity of PAP to PAPS used as a coenzyme cofactor for other sulfotransferase activities. The mutated arylsulfotransferases can be advantageously used in sulfation bioprocess systems for reducing the inhibitory effects of PAP accumulation on other sulfotransferase activity while also constantly feed the system with the primary sulfur donor molecule, PAPS.

The mutated arylsulfotransferases as disclosed herein can be advantageously used in heparin synthesis bioprocess systems for enhancing the conversion activity of PAP to PAPS used as a coenzyme cofactor in a recycling system involving other sulfotransferase activities. In other words, the mutated arylsulfotransferases as disclosed herein can be advantageously used in heparin synthesis bioprocess systems for reducing the inhibitory effects of PAP accumulation on other sulfotransferase activity while also constantly feed the system with the primary sulfur donor molecule, PAPS.

The present disclosure provides advantageously a source of PAPS at low cost and high yield, allowing the large-scale synthesis of sulfated substrate such as heparan sulfate and heparin.

Furthermore, the present disclosure provides mutated arylsulfotransferases with enhanced activity to convert PAP into PAPS which can be easily recombinantly obtained.

The mutated non-naturally occurring arylsulfotransferases disclosed herein have an enhanced thermal and/or structural stability resulting in a more sustainable and/or enhanced catalytic activity.

The present disclosure provides advantageously methods for obtaining sulfated substrate, such as heparan sulfate and heparin, at high-yield and low cost, allowing an efficient industrial scale-up.

A non-naturally occurring mutated arylsulfotransferase disclosed herein may have a sulfotransferase activity for converting adenosine 3′,5′-bisphosphate (PAP) into 3′-phosphoadenosine-5′-phosphosulfate (PAPS) at least 1.3 times greater than the said activity of the rat arylsulfotransferase IV of SEQ ID NO: 1. The increase of activity of non-naturally occurring mutated arylsulfotransferase of at least about 1.3 activity times compared with the activity of the rat arylsulfotransferase IV of SEQ ID NO: 1 may be measured with a colorimetric method as described hereafter.

According to one of its object, the present invention relates to a non-naturally occurring mutated arylsulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, (ii) an amino acid sequence having at least 60% sequence identity with SEQ ID NO: 1, and (iii) having a sulfotransferase activity for converting adenosine 3′,5′-bisphosphate (PAP) into 3′-phosphoadenosine-5′-phosphosulfate (PAPS) at least 1.3 times greater than the said activity of the rat arylsulfotransferase IV of SEQ ID NO: 1. The increase of activity of non-naturally occurring mutated arylsulfotransferase of at least about 1.3 activity times compared with the activity of the rat arylsulfotransferase IV of SEQ ID NO: 1 may be measured with a colorimetric method as described hereafter.

According to one of its object, the present invention relates to a non-naturally occurring mutated arylsulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, (ii) an amino acid sequence having at least 60% sequence identity with SEQ ID NO: 1, and (iii) having a sulfotransferase activity for converting adenosine 3′,5′-bisphosphate (PAP) into 3′-phosphoadenosine-5′-phosphosulfate (PAPS) at least substantially similar to or greater than the said activity of a non-naturally occurring mutated arylsulfotransferase having an amino acid sequence selected among SEQ ID NO: 5 to 23, 25-35, 41, 45-47, and 49-56.

A non-naturally occurring mutated arylsulfotransferase as disclosed herein may comprise an amino acid substitution in at least 2, or at least 3, or at least 4, or at least 5, or at least 6, or at least 7, or at least 8, or at least 9, or 10 amino acid positions selected among positions 6, 7, 8, 9, 11, 33, 62, 97, 195, and/or 263.

A non-naturally occurring mutated arylsulfotransferase may comprise an amino acid substitution in no more than 2, or no more than 3, or no more than 4, or no more than 5, or no more than 6, or no more than 7, or no more than 8, or no more than 9 amino acids positions selected among positions 6, 7, 8, 9, 11, 33, 62, 97, 195, and/or 263.

A non-naturally occurring mutated arylsulfotransferase may comprise an amino acid substitution in the amino acid positions 6, 7, 8, 9, and 11. In such embodiment, a non-naturally occurring mutated arylsulfotransferase may further comprise an amino acid substitution in at least one amino acid position selected among positions 33, 62, 97, 195, and/or 263.

A non-naturally occurring mutated arylsulfotransferase may comprise an amino acid substitution in the amino acid positions 33, 62, 97, 195, and 263. In such embodiment, a non-naturally occurring mutated arylsulfotransferase may further comprise an amino acid substitution in at least one amino acid position selected among positions 6, 7, 8, 9, and/or 11.

A non-naturally occurring mutated arylsulfotransferase may comprise an amino acid substitution in the amino acid positions 6, 7, 8, 9, 11, 33, 62, 97, 195, and 263, and optionally in position 236.

A non-naturally occurring mutated arylsulfotransferase may comprise an amino acid substitution in the amino acid positions 6, 7, 8, 9, 11, 33, 62, 97, 263, and 236. In such embodiment, a non-naturally occurring mutated arylsulfotransferase may not comprise an amino acid substitution in the amino acid position 195.

A non-naturally occurring mutated arylsulfotransferase may further comprise an amino acid substitution in at least 1, or at least 2, or at least 3, or at least 4, or at least 5, or 6 amino acid position selected among positions 17, 20, 138, 236, 239, and/or 244.

A non-naturally occurring mutated arylsulfotransferase may further comprise an amino acid substitution in no more than 1, or no more than 2, or no more than 3, or no more than 4, or no more than 5 amino acid position(s) selected among positions 17, 20, 138, 236, 239, and/or 244.

A non-naturally occurring mutated arylsulfotransferase may comprise as substituting amino acid:
in position 6 a glutamine (Q), or an asparagine (N), and in some embodiments, a substituting amino acid in position 6 may be a glutamine (Q),
in position 7 an aspartate (D), or a glutamate (E), and in some embodiments, a substituting amino acid in position 7 may be an aspartate (D),
in position 8 an alanine (A), a glycine (G), or a valine (V), and in some embodiments, a substituting amino acid in position 8 may be an alanine (A),
in position 9 a glycine (G), an alanine (A) or a valine (V), and in some embodiments, a substituting amino acid in position 9 may be a glycine (G),
in position 11 a leucine (L), a valine (V) or an isoleucine (1), and in some embodiments, a substituting amino acid in position 11 may be a leucine (L),
in position 17 a phenylalanine (F) or a tyrosine (Y),
in position 20 an isoleucine (1) or a leucine (L),
in position 33 an arginine (R), an histidine (H) or a lysine (K), and in some embodiments, a substituting amino acid in position 33 may be an arginine (R),
in position 62 an aspartate (D), or a glutamate (E), and in some embodiments, a substituting amino acid in position 62 may be an aspartate (D),
in position 97 a serine (S), or a threonine (T), and in some embodiments, a substituting amino acid in position 97 may be a serine (S),
in position 138 an histidine (H), a lysine (K) or an arginine (R), and in some embodiments, a substituting amino acid in position 138 may be an histidine (H),
in position 195 an aspartate (D), or a glutamate (E), and in some embodiments, a substituting amino acid in position 195 may be an aspartate (D),
in position 236 a phenylalanine (F), or a tryptophan (W), and in some embodiments, a substituting amino acid in position 236 may be a phenylalanine (F),
in position 239 an aspartate (D), or a glutamate (E), and in some embodiments, a substituting amino acid in position 239 may be an aspartate (D),
in position 244 an asparagine (N), or a glutamine (Q), and/or and in some embodiments, a substituting amino acid in position 244 may be an asparagine (N),
in position 263 an histidine (H), a lysine (K) or an arginine (R), and in some embodiments, a substituting amino acid in position 263 may be an histidine (H).

A non-naturally occurring mutated arylsulfotransferase may comprise at least one amino acid substitution selected among P60, P7D, L8A, V9G, V11L, I17F, I17Y, F20L, F20I, W33R, K62D, A97S, F138H, N195D, Y236F, I239D, M244N, T263H, and combinations thereof.

A non-naturally occurring mutated arylsulfotransferase may comprise at least the amino acid substitution P60.

A non-naturally occurring mutated arylsulfotransferase may further comprise an amino acid substitution selected among W33R, K62D, and combination thereof.

A non-naturally occurring mutated arylsulfotransferase may comprise the amino acid substitutions W33R, K62D, A97S, N195D, and T263H. In such embodiment, a non-naturally occurring mutated arylsulfotransferase may further comprise at least one amino acid substitution selected among P60, P7D, L8A, V9G, V11L, and combinations thereof.

A non-naturally occurring mutated arylsulfotransferase may comprise at least the amino acid substitutions P60, P7D, L8A, V9G, V11L, W33R, K62D, A97S, N195D, and T263H.

A non-naturally occurring mutated arylsulfotransferase may comprise at least the amino acid substitutions P60, P7D, L8A, V9G, V11L, W33R, K62D, A97S, and T263H. Optionally, a non-naturally occurring mutated arylsulfotransferase does not comprise the substitution N195D.

A non-naturally occurring mutated arylsulfotransferase may further comprise the amino acid substitution Y236F.

A non-naturally occurring mutated arylsulfotransferase may comprise at least, or may comprise only, the amino acid substitutions P60, P7D, L8A, V9G, V11L, W33R, K62D, A97S, Y236F, and T263H.

A non-naturally occurring mutated arylsulfotransferase may have an amino acids sequence selected among SEQ ID NO: 5 to 23, 25-35, 41, 45-47, and 49-56. A non-naturally occurring mutated arylsulfotransferase may have an amino acids sequence having at least 60% identity with a sequence selected among SEQ ID NO: 5 to 23, 25-35, 41, 45-47, and 49-56 and a sulfotransferase activity for converting adenosine 3′,5′-bisphosphate (PAP) into 3′-phosphoadenosine-5′-phosphosulfate (PAPS) at least about 1.3 times greater than the said activity of the rat arylsulfotransferase IV of SEQ ID NO: 1. The increase of activity of non-naturally occurring mutated arylsulfotransferase of at least about 1.3 activity times compared with the activity of the rat arylsulfotransferase IV of SEQ ID NO: 1 may be measured with a colorimetric method as described hereafter.

A non-naturally occurring mutated arylsulfotransferase may have an amino acids sequence having at least 60% identity with a sequence selected among SEQ ID NO: 5 to 23, 25-35, 41, 45-47, and 49-56 and a sulfotransferase activity for converting adenosine 3′,5′-bisphosphate (PAP) into 3′-phosphoadenosine-5′-phosphosulfate (PAPS) substantially similar or greater than the said activity of a non-naturally occurring mutated arylsulfotransferase having an amino acid sequence selected among SEQ ID NO: 5 to 23, 25-35, 41, 45-47, and 49-56.

A non-naturally occurring mutated arylsulfotransferase may have a sulfotransferase activity for converting adenosine 3′,5′-bisphosphate (PAP) into 3′-phosphoadenosine-5′-phosphosulfate (PAPS) being at least 1.5 times, or at least 1.8, or at least 1.9, or at least 2.0, or at least 2.2, or at least 2.5, or at least 3.0, or at least 3.2, or at least 3.5, or at least 4.0, or at least 4.5, or at least 5.0, or at least 5.5, or at least 6.0, or at least 6.5, or at least 7.0 times greater than of the said activity of the rat arylsulfotransferase IV of SEQ ID NO: 1.

According to one of its objects, the present invention relates to an isolated nucleic acid encoding a non-naturally occurring mutated arylsulfotransferase as disclosed herein.

According to one of its objects, the present invention relates to a recombinant expression vector comprising a nucleic acid as disclosed herein.

According to one of its objects, the present invention relates an in vitro or a recombinant host cell comprising a nucleic acid or a recombinant expression vector as disclosed herein.

According to one of its objects, the present invention relates a kit for sulfating a substrate, the kit comprising at least:

one non-naturally occurring mutated arylsulfotransferase as disclosed herein in a first container; and

a sulfo group donor in a second container.

In some embodiments, in a kit as disclosed herein a sulfo group donor may be an aryl sulfate compound.

In some embodiments, in a kit as disclosed herein the aryl sulfate compound is p-Nitrophenyl sulfate (pNPS).

In some embodiments, a kit as disclosed herein may further comprise a buffer.

In some embodiments, in a kit as disclosed herein the buffer may be selected in the group comprising TRIS-buffer, sodium phosphate buffer, and potassium phosphate buffer.

According to one of its objects, the present invention relates a method of selecting a non-naturally occurring mutated arylsulfotransferase comprising at least one amino acid substitution and comprising a sulfotransferase activity for converting adenosine 3′,5′-bisphosphate (PAP) into 3′-phosphoadenosine-5′-phosphosulfate (PAPS) being at least 1.3 times greater than the said activity of the rat arylsulfotransferase IV of SEQ ID NO: 1 or being at least substantially the same or greater than said activity of a non-naturally occurring mutated arylsulfotransferase having an amino acids sequence selected in the group comprising SEQ ID NO: 5 to 23, 25-35,41, 45-47, and 49-56, said method comprising at least the steps of:

a) contacting a non-naturally occurring mutated arylsulfotransferase candidate comprising at least one amino acid substitution with a sulfo group donor in conditions suitable for a transfer of the sulfo group from the sulfo group donor to PAP to obtain PAPS,

b) detecting a rate or an amount of formation of PAPS,

c) comparing the rate or amount of formation of PAPS obtained at step b) with a rate or an amount of reference obtained with a rat arylsulfotransferase IV of SEQ ID NO: 1 or obtained with a non-naturally occurring mutated arylsulfotransferase having an amino acids sequence selected in the group comprising SEQ ID NO: 5 to 23, 25-35, 41, 45-47, and 49-56, and

d) selecting any non-naturally occurring mutated arylsulfotransferase candidate comprising at least one amino acid substitution and comprising a sulfotransferase activity for converting adenosine 3′,5′-bisphosphate (PAP) into 3′-phosphoadenosine-5′-phosphosulfate (PAPS) being at least 1.3 times greater than the said activity of the rat arylsulfotransferase IV of SEQ ID NO: 1 or being at least substantially the same or greater than said activity of a non-naturally occurring mutated arylsulfotransferase having an amino acids sequence selected in the group comprising SEQ ID NO: 5 to 23, 25-35, 41, 45-47, and 49-56.

The increase of activity of non-naturally occurring mutated arylsulfotransferase of at least about 1.3 activity times compared with the activity of the rat arylsulfotransferase IV of SEQ ID NO: 1 may be measured with a colorimetric method as described hereafter.

In some embodiments, in a method as disclosed herein a sulfo group donor may be p-nitrophenyl sulfate.

According to one of its objects, the present invention relates a non-naturally occurring mutated arylsulfotransferase comprising at least one amino acid substitution and comprising a sulfotransferase activity for converting adenosine 3′,5′-bisphosphate (PAP) into 3′-phosphoadenosine-5′-phosphosulfate (PAPS) being at least 1.3 times greater than the said activity of the rat arylsulfotransferase IV of SEQ ID NO: 1 or being at least substantially the same or greater than said activity of a non-naturally occurring mutated arylsulfotransferase having an amino acids sequence selected in the group comprising SEQ ID NO: 5 to 23, 25-35, 41, 45-47, and 49-56 identified by a method as disclosed herein. The increase of activity of non-naturally occurring mutated arylsulfotransferase of at least about 1.3 activity times compared with the activity of the rat arylsulfotransferase IV of SEQ ID NO: 1 may be measured with a colorimetric method as described hereafter.

According to one of its objects, the present invention relates a use of a non-naturally occurring mutated arylsulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, and (ii) an amino acid sequence having at least 60% sequence identity with amino acids sequence SEQ ID NO: 1, with the proviso that when said arylsulfotransferase is the rat arylsulfotransferase IV, the mutations are not F138A and/or Y236A, for sulfating a substrate.

In a use as disclosed herein a non-naturally occurring mutated arylsulfotransferase may have a sulfotransferase activity for converting adenosine 3′,5′-bisphosphate (PAP) into 3′-phosphoadenosine-5′-phosphosulfate (PAPS) at least 1.3 times greater than the said activity of the rat arylsulfotransferase IV of SEQ ID NO: 1. The increase of activity of non-naturally occurring mutated arylsulfotransferase of at least about 1.3 activity times compared with the activity of the rat arylsulfotransferase IV of SEQ ID NO: 1 may be measured with a colorimetric method as described hereafter.

According to one of its objects, the present invention relates a use of a non-naturally occurring mutated arylsulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, (ii) an amino acid sequence having at least 60% sequence identity with amino acids sequence SEQ ID NO: 1, and (iii) having a sulfotransferase activity for converting adenosine 3′,5′-bisphosphate (PAP) into 3′-phosphoadenosine-5′-phosphosulfate (PAPS) at least 1.3 times greater than the said activity of the rat arylsulfotransferase IV of SEQ ID NO: 1, for sulfating a substrate. The increase of activity of non-naturally occurring mutated arylsulfotransferase of at least about 1.3 activity times compared with the activity of the rat arylsulfotransferase IV of SEQ ID NO: 1 may be measured with a colorimetric method as described hereafter.

Alternatively, in the uses as disclosed herein a non-naturally occurring mutated arylsulfotransferase may have a sulfotransferase activity for converting adenosine 3′,5′-bisphosphate (PAP) into 3′-phosphoadenosine-5′-phosphosulfate (PAPS) substantially similar or greater than the said activity of a non-naturally occurring mutated arylsulfotransferase having an amino acid sequence selected among SEQ ID NO: 5 to 23, 25-35, 41, 45-47, and 49-56.

According to one of its objects, the present invention relates a method for sulfating a substrate, comprising at least a step of contacting said substrate to be sulfated with:

a) a non-naturally occurring mutated arylsulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, and (ii) an amino acid sequence having at least 60% sequence identity with amino acids sequence SEQ ID NO: 1, with the proviso that when said arylsulfotransferase is the rat arylsulfotransferase IV, the mutations are not F138A and/or Y236A, and

b) a sulfo group donor in conditions suitable for a transfer of the sulfo group from the sulfo group donor to said substrate.

According to one of its objects, the present invention relates a method for sulfating a substrate, comprising at least a step of contacting said substrate to be sulfated with:

a) a non-naturally occurring mutated arylsulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, (ii) an amino acid sequence having at least 60% sequence identity with amino acids sequence SEQ ID NO: 1 and (iii) having a sulfotransferase activity for converting adenosine 3′,5′-bisphosphate (PAP) into 3′-phosphoadenosine-5′-phosphosulfate (PAPS) at least 1.3 times the said activity of the rat arylsulfotransferase IV of SEQ ID NO: 1, and

b) a sulfo group donor in conditions suitable for a transfer of the sulfo group from the sulfo group donor to said substrate. The increase of activity of non-naturally occurring mutated arylsulfotransferase of at least about 1.3 activity times compared with the activity of the rat arylsulfotransferase IV of SEQ ID NO: 1 may be measured with a colorimetric method as described hereafter.

Alternatively, in the methods as disclosed herein a non-naturally occurring mutated arylsulfotransferase may have a sulfotransferase activity for converting adenosine 3′,5′-bisphosphate (PAP) into 3′-phosphoadenosine-5′-phosphosulfate (PAPS) substantially similar or greater than the said activity of a non-naturally occurring mutated arylsulfotransferase having an amino acid sequence selected among SEQ ID NO: 5 to 23, 25-35, 41, 45-47, and 49-56.

According to one of its objects, the present invention relates a method for sulfating a substrate with a sulfotransferase and PAPS in conditions suitable to transfer a sulfo group from PAPS to the substrate to be sulfated and to obtain a sulfated substrate and PAP, comprising at least a step of converting the PAP so-obtained into PAPS by contacting the PAP with:

(i) a non-naturally occurring mutated arylsulfotransferase comprising (1) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, and (2) an amino acid sequence having at least 60% sequence identity with amino acids sequence SEQ ID NO: 1, and

(ii) a sulfo group donor

in conditions suitable for a transfer of the sulfo group from the sulfo group donor to PAP to obtain PAPS.

In the uses or methods as disclosed herein a non-naturally occurring mutated arylsulfotransferase may have a sulfotransferase activity for converting adenosine 3′,5′-bisphosphate (PAP) into 3′-phosphoadenosine-5′-phosphosulfate (PAPS) at least 1.3 times the said activity of the rat arylsulfotransferase IV of SEQ ID NO: 1. The increase of activity of non-naturally occurring mutated arylsulfotransferase of at least about 1.3 activity times compared with the activity of the rat arylsulfotransferase IV of SEQ ID NO: 1 may be measured with a colorimetric method as described hereafter.

Alternatively, in the uses or methods as disclosed herein a non-naturally occurring mutated arylsulfotransferase may have a sulfotransferase activity for converting adenosine 3′,5′-bisphosphate (PAP) into 3′-phosphoadenosine-5′-phosphosulfate (PAPS) substantially similar or greater than the said activity of a non-naturally occurring mutated arylsulfotransferase having an amino acid sequence selected among SEQ ID NO: 5 to 23, 25-35, 41, 45-47, and 49-56.

In a method as disclosed herein a substrate may be sulfated with one or a plurality of sulfotransferases to carry out a plurality of sulfation.

In a method as disclosed herein a plurality of sulfation may be carried out concomitantly or sequentially.

In a method as disclosed herein a step of converting PAP into PAPS may be carried out concomitantly with the sulfation or separately.

In a method as disclosed herein a step of sulfation and a step of converting PAP into PAPS may be carried out concomitantly in a same reaction mixture.

A method as disclosed herein may further comprise a step of recovering the sulfated substrate.

In uses or methods as disclosed herein a substrate may be selected in a group comprising adenosine 3′,5′-bisphosphate (PAP), a polysaccharide, an heparan, an heparosan sulfate, a chemically desulfated N-sulfated (CDSNS) heparin, a glycosaminoglycan (GAG), an heparan sulfate or a sulfated heparin.

A use or a method as disclosed herein may be for converting adenosine 3′,5′-bisphosphate (PAP) into 3′-phosphoadenosine-5′-phosphosulfate (PAPS).

A use or a method as disclosed herein may be for preparing a heparin.

According to one of its objects, the present invention relates a method for recycling PAP into PAPS, comprising at least a step of contacting said PAP with:

a) a non-naturally occurring mutated aryl sulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, and (ii) an amino acid sequence having at least 60% sequence identity with amino acids sequence SEQ ID NO: 1, and

b) a sulfo group donor in conditions suitable for a transfer of the sulfo group from the sulfo group donor to PAP to obtain PAPS.

In a method as disclosed herein a sulfo group donor may be an aryl sulfate compound.

An aryl sulfate compound may be p-Nitrophenyl sulfate (pNPS).

In uses or methods as disclosed herein a mutated non-naturally occurring arylsulfotransferase may be grafted onto a support.

In uses or methods as disclosed herein a non-naturally occurring mutated arylsulfotransferase may have an amino acids sequence selected among SEQ ID NO: 5 to 23, 25-35, 41, 45-47, and 49-56.

In uses or methods as disclosed herein a non-naturally occurring mutated arylsulfotransferase may have an amino acids sequence having at least 60% identity with a sequence selected among SEQ ID NO: 5 to 23, 25-35, 41, 45-47, and 49-56 and a sulfotransferase activity for converting adenosine 3′,5′-bisphosphate (PAP) into 3′-phosphoadenosine-5′-phosphosulfate (PAPS) at least about 1.3 times greater than the said activity of the rat arylsulfotransferase IV of SEQ ID NO: 1. The increase of activity of non-naturally occurring mutated arylsulfotransferase of at least about 1.3 activity times compared with the activity of the rat arylsulfotransferase IV of SEQ ID NO: 1 may be measured with a colorimetric method as described hereafter.

Alternatively, in the uses or methods as disclosed herein a non-naturally occurring mutated arylsulfotransferase may have an amino acids sequence having at least 60% identity with a sequence selected among SEQ ID NO: 5 to 23, 25-35, 41, 45-47, and 49-56 and a sulfotransferase activity for converting adenosine 3′,5′-bisphosphate (PAP) into 3′-phosphoadenosine-5′-phosphosulfate (PAPS) at least substantially similar or greater than the said activity of a non-naturally occurring mutated arylsulfotransferase having an amino acid sequence selected among SEQ ID NO: 5 to 23, 25-35, 41, 45-47, and 49-56.

### DESCRIPTION OF THE FIGURES

FIGS. 1A-1B: FIG. 1A represents the rat AST IV sulfation activity converting PAP in PAPS measured by absorbance at 404 nm on pNP production and obtained with wild-type (AST IV) and mutants Var01 to Var09 10 minutes after initiation of the reaction. FIG. 1B represents the rat AST IV sulfation activity converting PAP in PAPS and measured by absorbance at 404 nm on pNP production and obtained with wild-type (AST IV) and mutants Var01 to Var09 at 30 minutes after initiation of the reaction.

FIG. 2: represents the rat AST IV sulfation activity converting PAP in PAPS measured by absorbance at 404 nm on pNP production and obtained with wild-type (AST IV) and mutants Var09−1 to Var09−10, and Var09 at 90 minutes after initiation of the reaction

FIG. 3: represents the rat AST IV sulfation activity converting PAP in PAPS measured by absorbance at 404 nm on pNP production and obtained with wild-type (AST IV) and mutants Var09−P6Q, Var09−P7D, Var09−L8A, Var09−V9G, Var09−V11L, Var09−W33R, Var09−K62D, Var09−A97S, Var09−N195D, Var09−T263H, VAR09−K62D-T263H, VAR09−K62D-N195D-T263H, and Var09 at 10 minutes after initiation of the reaction.

FIG. 4: represents the rat AST IV sulfation activity converting PAP in PAPS measured by absorbance at 404 nm on pNP production and obtained with wild-type (AST IV) and mutants Var09+I17F, Var09+I17Y, Var09+F20I, Var09+F20L, Var09+F138H, Var09+Y236F, Var09+I239D, Var09+M244N, and Var09 at 10 minutes after initiation of the reaction.

FIG. 5: represents the rat AST IV sulfation activity converting PAP in PAPS measured by absorbance at 404 nm on pNP production and obtained with wild-type (AST IV) and mutants Var09, Var5A (P6Q, P7D, L8A, V9G, V11L), Var5B (W33R, K62D, A97S, N195D, T263H), Var5A+W33R, Var5A+K62D, Var5A+A97S, Var5A+N195D, Var5A+T263H, Var5B+P6Q, Var5B+P7D, Var5B+L8A, Var5B+V9G, and Var5B+V11L at 10 minutes after initiation of the reaction.

FIG. 6: represents the alignment of sequences or the arylsulfotransferase (AST) from Gallus gallus (SEQ ID NO: 3), Rattus norvegicus (SEQ ID NO: 1), Homo sapiens (SEQ ID NO: 2), and Bos taurus (SEQ ID NO: 4). In the AST sequence from the rat are indicated in bold and underlined the positions which can be mutated by amino acid substitution.

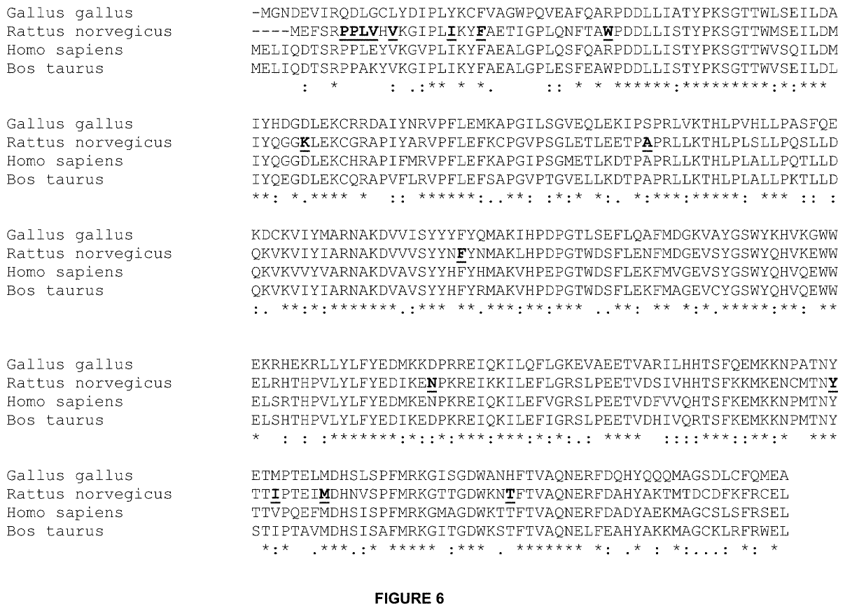

FIGS. 7A-7B: represent 2-O sulfation activities on N-Sulfated heparosan (NS heparosan) in presence of C5-epimerase and different AST-IV WT and variants [“Var09” (SEQ ID NO: 13), “Var09−N195D” (SEQ ID NO: 32), and “Var09+Y236F” (SEQ ID NO: 41)] on two experiments using two different AST-IV enzyme quantities, respectively 0.1 g/L (FIG. 7A) and 0.03 g/L (FIG. 7B).

### DESCRIPTION OF THE SEQUENCES

SEQUENCE ID NO: 1 represents the amino acids sequence of the rat arylsulfotransferase IV.

SEQUENCE ID NO: 2 represents the amino acids sequence of the arylsulfotransferase from Homo sapiens.

SEQUENCE ID NO: 3 represents the amino acids sequence of the arylsulfotransferase from Gallus gallus.

SEQUENCE ID NO: 4 represents the amino acids sequence of the arylsulfotransferase from Bos taurus.

SEQUENCE ID NO: 5 represents the amino acids sequence of the rat arylsulfotransferase IV comprising the mutation I17F (Var01).

SEQUENCE ID NO: 6 represents the amino acids sequence of the rat arylsulfotransferase IV comprising the mutation F20L (Var04).

SEQUENCE ID NO: 7 represents the amino acids sequence of the rat arylsulfotransferase IV comprising the mutation F20I (Var03).

SEQUENCE ID NO: 8 represents the amino acids sequence of the rat arylsulfotransferase IV comprising the mutation F138H (Var05).

SEQUENCE ID NO: 9 represents the amino acids sequence of the rat arylsulfotransferase IV comprising the mutation Y236F (Var06).

SEQUENCE ID NO: 10 represents the amino acids sequence of the rat arylsulfotransferase IV comprising the mutation M244N (Var07).

SEQUENCE ID NO: 11 represents the amino acids sequence of the rat arylsulfotransferase IV comprising the mutation I17Y (Var02).

SEQUENCE ID NO: 12 represents the amino acids sequence of the rat arylsulfotransferase IV comprising the mutation I239D (Var08).

SEQUENCE ID NO: 13 represents the amino acids sequence of the rat arylsulfotransferase IV comprising the mutations P60, P7D, L8A, V9G, V11L, W33R, K62D, A97S, N195D, and T263H (Var09).

SEQUENCE ID NO: 14 represents the amino acids sequence of the rat arylsulfotransferase IV comprising the mutations P60 (Var09−1).

SEQUENCE ID NO: 15 represents the amino acids sequence of the rat arylsulfotransferase IV comprising the mutations P7D (Var09−2).

SEQUENCE ID NO: 16 represents the amino acids sequence of the rat arylsulfotransferase IV comprising the mutations L8A (Var09−3).

SEQUENCE ID NO: 17 represents the amino acids sequence of the rat arylsulfotransferase IV comprising the mutations V9G (Var09−4).

SEQUENCE ID NO: 18 represents the amino acids sequence of the rat arylsulfotransferase IV comprising the mutations V11L (Var09−5).

SEQUENCE ID NO: 19 represents the amino acids sequence of the rat arylsulfotransferase IV comprising the mutations W33R (Var09−6).

SEQUENCE ID NO: 20 represents the amino acids sequence of the rat arylsulfotransferase IV comprising the mutations K62D (Var09−7).

SEQUENCE ID NO: 21 represents the amino acids sequence of the rat arylsulfotransferase IV comprising the mutations A97S (Var09−8).

SEQUENCE ID NO: 22 represents the amino acids sequence of the rat arylsulfotransferase IV comprising the mutations N195D (Var09−9).

SEQUENCE ID NO: 23 represents the amino acids sequence of the rat arylsulfotransferase IV comprising the mutations T263H (Var09−10).

SEQUENCE ID NO: 24 represents the amino acids sequence of the rat arylsulfotransferase IV comprising the mutations P7D-L8A-V9G-V11L-W33R-K62D-A97S-N195D-T263H (Var09 less mutation P6Q: “Var09−P6Q”).

SEQUENCE ID NO: 25 represents the amino acids sequence of the rat arylsulfotransferase IV comprising the mutations P6Q-L8A-V9G-V11L-W33R-K62D-A97S-N195D-T263H (Var09 less mutation P7D: “Var09−P7D”).

SEQUENCE ID NO: 26 represents the amino acids sequence of the rat arylsulfotransferase IV comprising the mutations P6Q-P7D-V9G-V11L-W33R-K62D-A97S-N195D-T263H (Var09 less mutation L8A: “Var09−L8A”).

SEQUENCE ID NO: 27 represents the amino acids sequence of the rat arylsulfotransferase IV comprising the mutations P6Q-P7D-L8A-V11L-W33R-K62D-A97S-N195D-T263H (Var09 less mutation V9G: “Var09−V9G”).

SEQUENCE ID NO: 28 represents the amino acids sequence of the rat arylsulfotransferase IV comprising the mutations P6Q-P7D-L8A-V9G-W33R-K62D-A97S-N195D-T263H (Var09 less mutation V11L: “V11L”).

SEQUENCE ID NO: 29 represents the amino acids sequence of the rat arylsulfotransferase IV comprising the mutations P6Q-P7D-L8A-V9G-V11L-A97S-N195D-T263H (Var09 less mutation W33R: “Var09−W33R”).

SEQUENCE ID NO: 30 represents the amino acids sequence of the rat arylsulfotransferase IV comprising the mutations P6Q-P7D-L8A-V9G-V11 L-W33R-A97S-N195D-T263H (Var09 less mutation K62D: “Var09−K62D”).

SEQUENCE ID NO: 31 represents the amino acids sequence of the rat arylsulfotransferase IV comprising the mutations P6Q-P7D-L8A-V9G-V11L-W33R-K62D-N195D-T263H (Var09 less mutation A97S: “Var09−A97S”).

SEQUENCE ID NO: 32 represents the amino acids sequence of the rat arylsulfotransferase IV comprising the mutations P6Q-P7D-L8A-V9G-V11L-W33R-K62D-A97S-T263H (Var09 less mutation N195D: “Var09−N195D”).

SEQUENCE ID NO: 33 represents the amino acids sequence of the rat arylsulfotransferase IV comprising the mutations P6Q-P7D-L8A-V9G-V11L-W33R-K62D-A97S-N195D (Var09 less mutation T263H: “Var09−T263H”).

SEQUENCE ID NO: 34 represents the amino acids sequence of the rat arylsulfotransferase IV comprising the mutations P6Q-P7D-L8A-V9G-V11L-W33R-A97S-N195D (Var09 less mutations K62D and T263H: “Var09−K62D-T263H”).

SEQUENCE ID NO: 35 represents the amino acids sequence of the rat arylsulfotransferase IV comprising the mutations P6Q-P7D-L8A-V9G-V11L-W33R-A97S (“Var09 less mutations K62D, N195D and T263H:Var09−K62D-N195D-T263H”).

SEQUENCE ID NO: 36 represents the amino acids sequence of the rat arylsulfotransferase IV comprising the mutations P60, P7D, L8A, V9G, V11L, I17F, W33R, K62D, A97S, N195D, and T263H (Var09 plus mutation I17F: “Var09+I17F”).

SEQUENCE ID NO: 37 represents the amino acids sequence of the rat arylsulfotransferase IV comprising the mutations P60, P7D, L8A, V9G, V11L, I17Y, W33R, K62D, A97S, N195D, and T263H (Var09 plus mutation I17Y: “Var09+I17Y”).

SEQUENCE ID NO: 38 represents the amino acids sequence of the rat arylsulfotransferase IV comprising the mutations P60, P7D, L8A, V9G, V11L, F20I, W33R, K62D, A97S, N195D, and T263H (“Var09 plus mutation F20I: Var09+F20I”).

SEQUENCE ID NO: 39 represents the amino acids sequence of the rat arylsulfotransferase IV comprising the mutations P60, P7D, L8A, V9G, V11L, F20L, W33R, K62D, A97S, N195D, and T263H (Var09 plus mutation F20L: “Var09+F20L”).

SEQUENCE ID NO: 40 represents the amino acids sequence of the rat arylsulfotransferase IV comprising the mutations P60, P7D, L8A, V9G, V11L, W33R, K62D, A97S, F138H, N195D, and T263H (Var09 plus mutation F138H: “Var09+F138H”).

SEQUENCE ID NO: 41 represents the amino acids sequence of the rat arylsulfotransferase IV comprising the mutations P60, P7D, L8A, V9G, V11L, W33R, K62D, A97S, N195D, Y236F, and T263H (Var09 plus mutation Y236F: “Var09+Y236F”).

SEQUENCE ID NO: 42 represents the amino acids sequence of the rat arylsulfotransferase IV comprising the mutations P60, P7D, L8A, V9G, V11L, W33R, K62D, A97S, N195D, I239D, and T263H (Var09 plus mutation I239D: “Var09+I239D”).

SEQUENCE ID NO: 43 represents the amino acids sequence of the rat arylsulfotransferase IV comprising the mutations P60, P7D, L8A, V9G, V11L, W33R, K62D, A97S, N195D, M244N, and T263H (“Var09 plus mutation M244N: Var09+M244N”).

SEQUENCE ID NO: 44 represents the amino acids sequence of the rat arylsulfotransferase IV comprising the mutations P60, P7D, L8A, V9G, and V11L, (“Var5A”).

SEQUENCE ID NO: 45 represents the amino acids sequence of the rat arylsulfotransferase IV comprising the mutations W33R, K62D, A97S, N195D, and T263H (“Var5B”).

SEQUENCE ID NO: 46 represents the amino acids sequence of the rat arylsulfotransferase IV comprising the mutations P60, P7D, L8A, V9G, V11L, and W33R (“Var5A+W33R”).

SEQUENCE ID NO: 47 represents the amino acids sequence of the rat arylsulfotransferase IV comprising the mutations P60, P7D, L8A, V9G, V11L, and K62D (“Var5A+K62D”).

SEQUENCE ID NO: 48 represents the amino acids sequence of the rat arylsulfotransferase IV comprising the mutations P60, P7D, L8A, V9G, V11L, and A97S (“Var5A+A97S”).

SEQUENCE ID NO: 49 represents the amino acids sequence of the rat arylsulfotransferase IV comprising the mutations P60, P7D, L8A, V9G, V11L, and N195D (“Var5A+N195D”).

SEQUENCE ID NO: 50 represents the amino acids sequence of the rat arylsulfotransferase IV comprising the mutations P60, P7D, L8A, V9G, V11L, and T263H (“Var5A+T263H”).

SEQUENCE ID NO: 51 represents the amino acids sequence of the rat arylsulfotransferase IV comprising the mutations P60, W33R, K62D, A97S, N195D, and T263H (“Var5B+P6Q”).

SEQUENCE ID NO: 52 represents the amino acids sequence of the rat arylsulfotransferase IV comprising the mutations P7D, W33R, K62D, A97S, N195D, and T263H (“Var5B+P7D”).

SEQUENCE ID NO: 53 represents the amino acids sequence of the rat arylsulfotransferase IV comprising the mutations L8A, W33R, K62D, A97S, N195D, and T263H (“Var5B+L8A”).

SEQUENCE ID NO: 54 represents the amino acids sequence of the rat arylsulfotransferase IV comprising the mutations V9G, W33R, K62D, A97S, N195D, and T263H (“Var5B+V9G”).

SEQUENCE ID NO: 55 represents the amino acids sequence of the rat arylsulfotransferase IV comprising the mutations V11L, W33R, K62D, A97S, N195D, and T263H (“Var5B+V11L”).

SEQUENCE ID NO: 56 represents the amino acids sequence of the rat arylsulfotransferase IV comprising the mutations P60, P7D, L8A, V9G, V11L, W33R, K62D, A97S, T263H and Y236F (Var09 less N195D and plus Y236F: “Var09−N195D+Y236F”).

### DETAILED DESCRIPTION

### Definitions

It must be noted that as used herein and in the appended claims, the singular forms “a”, “an”, and “the” include plural referents unless the context clearly dictates otherwise. “A” and “an” mean “at least one”, unless the content clearly dictates otherwise

The terms “about” or “approximately” as used herein refer to the usual error range for the respective value readily known to the skilled person in this technical field. Reference to “about” a value or parameter herein includes (and describes) embodiments that are directed to that value or parameter per se. In some embodiments, the term “about” refers to ±10% of a given value. However, whenever the value in question refers to an indivisible object, such as a molecule or other object that would lose its identity once subdivided, then “about” refers to ±1 of the indivisible object.

Within the disclosure, the expressions “substitution” and “amino acid substitution” are used interchangeably and intend to refer to a substitution of one amino acid residue for another. An amino acid substitution may be conservative or not. A “conservative amino acid substitution” refers to a substitution of one amino acid residue for another sharing chemical and physical properties of the amino acid side chain, e.g., charge, size, hydrophobicity/hydrophilicity. An amino acid which is replaced by another is named a substituted amino acid. An amino acid replacing another one is named a substituting amino acid.

Within the disclosure, the expression “arylsulfotransferase” intends to refer to an enzyme that catalyzes the sulfate conjugation of a product. For example, an arylsulfotransferase may catalyze the transfer of a sulfo group on an aryl moiety, such as a phenol, in the presence of a sulfate donor (or sulfo donor), such as 3′-phosphoadenytylsulfate or 3′-phosphoadenosine-5′-phosphosulfate (PAPS), to yield an aryl sulfate and a metabolite of the sulfate donor, such as adenosine 3′,5′-bisphosphate or (PAP). In suitable conditions, the arylsulfotransferase may also catalyze the reverse of this reaction so as to generate PAPS from PAP.

Within the disclosure, the expression “arylsulfotransferase activity” intends to refer to the catalytic activity of an arylsulfotransferase transferring a sulfate group on PAP to generate PAPS. The sulfotransferase activity may result in the production of PAPS, the disappearance of PAP, the consumption of the sulfo donor group used in the reaction, or the production of the metabolite coming from the sulfo donor as a result of the reaction.

It is understood that aspects and embodiments of the present disclosure described herein include “having,” “comprising,” “consisting of,” and “consisting essentially of” aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic characteristic(s) of the disclosure. It is understood that the different embodiments of the disclosure using the term “comprising” or equivalent cover the embodiments where this term is replaced with “comprising only”, “consisting of” or “consisting essentially of”.

The expression “enhanced activity” with regard to a non-naturally occurring enzyme intends to mean that the enzyme has a catalytic activity, or a thermal stability or a structure stability which is enhanced compared to a wild-type enzyme.

Within the disclosure, the expression “Isolated” with regard to a compound or entity, such as an enzyme, refers to this compound or entity in an environment different from the one in which the compound or entity may naturally occur. “Isolated” is meant to include compound or entity in samples which are substantially enriched for this compound or entity and/or in which this compound or entity is partially or substantially purified. In some cases, an isolated compound or entity (e.g., a protein, such as a mutated arylsulfotransferase; a nucleic acid; a recombinant vector) is purified, e.g., it is at least 80%, at least 85%, at least 90%, at least 95%, at least 98%, at least 99%, or greater than 99%, pure.

Within the disclosure, the expression “non-naturally occurring” as used herein with regard to a nucleic acid, a peptide, polypeptide, or protein refers to any nucleic acid, peptide, polypeptide, or protein which are not found in nature.

Within the disclosure, the expression “mutant” as used herein with regard to a peptide, polypeptide, or protein refers to any peptide, polypeptide, or protein comprising at least one amino acid mutation. “Amino acid mutation” and “mutation” are used interchangeably and intend to refer to a substitution, a deletion, or an insertion of an amino acid, as compared to a wild-type, or naturally occurring, counterpart. In particular, a mutant peptide, polypeptide, or protein may comprise at least one amino acid substitution.

A “recombinant protein” as used herein intends to refer to a protein produced with a recombinant DNA. A “recombinant DNA” refers to a genetically-engineered DNA molecule formed by splicing fragments of DNA from different sources or from another part of the same source, and then introduced into the recipient (host) cell. For example, a recombinant protein may be produced by inserting the corresponding coding nucleic acid in a plasmid vector and delivering the vector in a host cell suitable for the expression of the protein.

Within the disclosure, the term “significantly” used with respect to change intends to mean that the observe change is noticeable and/or it has a statistic meaning.

Within the disclosure, the term “substantially” used in conjunction with a feature of the disclosure intends to define a set of embodiments related to this feature which are largely but not wholly similar to this feature. The difference between the set of embodiments related to the given feature and the given feature is such that in the set of embodiments, the nature and function of the given feature is not materially affected.

Within the disclosure, the expression “substantially the same or greater than” used to qualify the catalytic activity of a given enzyme with respect to the catalytic activity of a reference enzyme intends to define (i) that the catalytic activity of both enzymes, when measured with same protocol and conditions, are not significantly different or (ii) that the catalytic activity of the given enzyme is significantly above the catalytic activity of the reference enzyme, when both measured with same protocol and conditions. A catalytic activity significantly above a reference catalytic activity may be, for example, at least 1.3 times greater than the reference catalytic activity, for example at least 2-, 3- or 4-folds greater than the reference catalytic activity.

The term “sulfation” as used herein refers to a transfer of a sulfonate or sulfuryl group from one molecule to another.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” the list of items “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

Arylsulfotransferase Mutants

A non-naturally occurring mutated arylsulfotransferase as disclosed herein comprises, or consists of, an amino acids sequence which has at least 60% identity with the amino acids sequence SEQ ID NO:1 (the sequence of the rat arylsulfotransferase IV, or rat AST IV) and comprises an amino acid substitution in at least one amino acid position selected among positions 6, 7, 8, 9, 11, 17, 20, 33, 62, 97, 138, 195, 236, 239, 244, 263, and combinations thereof, the amino acid position being relative to the rat arylsulfotransferase IV of SEQ ID NO: 1.

In some embodiments, a non-naturally occurring mutated arylsulfotransferase as disclosed herein may be a rat arylsulfotransferase IV of SEQ ID NO: 1 comprising the amino acid substitutions and combinations thereof as disclosed herein.

In some embodiments, a non-naturally occurring mutated arylsulfotransferase as disclosed herein may comprise further mutations that the ones above indicated, provided that the additional mutations do not negatively affect the properties of the mutants disclosed herein, in particular the enhanced sulfation activity displayed compared to the sulfation activity of the rat arylsulfotransferase IV of SEQ ID NO: 1.

A non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least about 1.3 times greater than the said activity of the rat arylsulfotransferase IV of SEQ ID NO: 1.

An arylsulfotransferase activity may be detected and measured according to any known method in the art. In some embodiments, the increase of activity of non-naturally occurring mutated arylsulfotransferase of at least about 1.3 times compared with the activity of the rat arylsulfotransferase IV of SEQ ID NO: 1 may be measured with a colorimetric method. In some embodiments, the colometric method allows measuring the amount of p-Nitrophenyl (pNP) released (or produced) by the transfer of the sulfuryl group from p-Nitrophenyl sulfate (pNPS) to 3′,5′-adenosine-phosphate (PAP) for the production of 3′-phosphoadenosine-5′-phosphosulfate (PAPS) according to the following scheme reaction:

PAP+pNPS→PAPS+pNP

The method may comprise the steps of:

a) contacting a non-naturally occurring mutated arylsulfotransferase, for example expressed in bacteria or provided in a lysate of bacteria expressing said non-naturally occurring mutated arylsulfotransferase, or provided in a purified form, with a sufficient amount of pNPS and PAP, in a suitable buffer,

b) acquiring a measure representative of pNP produced at step a),

c) contacting a rat arylsulfotransferase IV of SEQ ID NO: 1, for example expressed in bacteria or provided in a lysate of bacteria expressing said non-naturally occurring mutated arylsulfotransferase, or provided in a purified form, with a sufficient amount of pNPS and PAP, in a suitable buffer,

d) acquiring a measure representative of pNP produced at step c), and

e) comparing the measures obtained at step b) and at step d).

Bacteria suitable for the expression of a mutated or a wild-type arylsulfotransferase (such as the rat arylsulfotransferase IV of SEQ ID NO: 1) may be E. coli BL21 DE3. The amount of enzyme suitable for the reaction, whatever the manner it is provided, may be of about 30 ng/μL.

Sufficient amounts of pNPS and PAP, when 30 ng/μL of enzyme are used, may be, respectively of about 1 mM and of about 0.23 mM.

A measure representative of pNP produced during the reaction may be obtained by a measure of the optical density at 404 nm, for example using a SpectraMax® 190 from Molecular Devices according to manufacturer's recommendations. The obtained measure may be expressed in arbitrary Unit of absorbance.

A suitable buffer for the reaction may be a phosphate buffer at pH 7.0 comprising glycerol at 10%.

A suitable temperature of reaction may be about 37° C.

The acquisition of the measure may be carried out 10, 30 or 90 minutes after initiation of the reaction, for example 10 minutes after initiation of the reaction.

In some embodiments, a blank may be subtracted to normalize the acquired measures. A blank may be water or a buffer without enzyme and substrates.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of anyone of the mutated arylsulfotransferases of SEQ ID NO: 5 to 23, 25-35, 41, 45-47, and 49-56.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 5.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 6.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 7.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 8.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 9.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 10.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 11.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 12.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 13.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 14.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 15.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 16.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 17.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 18.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 19.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 20.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 21.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 22.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 23.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 25.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 26.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 27.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 28.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 29.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 30.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 31.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 32.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 33.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 34.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 35.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 41.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 45.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 46.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 47.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 49.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 50.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 51.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 52.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 53.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 54.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 55.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 56.

A non-naturally occurring mutated arylsulfotransferase as disclosed herein comprises, or consists of, an amino acids sequence which has at least 60% identity with the amino acids sequence SEQ ID NO:1 (the sequence of the rat arylsulfotransferase IV, or rat AST IV) and comprises an amino acid substitution in at least one amino acid position selected among positions 6, 7, 8, 9, 11, 17, 20, 33, 62, 97, 138, 195, 236, 239, 244, 263, and combinations thereof, the amino acid position being relative to the rat arylsulfotransferase IV of SEQ ID NO: 1, and has a sulfotransferase activity for converting adenosine 3′,5′-bisphosphate (PAP) into 3′-phosphoadenosine-5′-phosphosulfate (PAPS) at least 1.3 times greater than the said activity of the rat arylsulfotransferase IV of SEQ ID NO: 1. The increase of activity of at least about 1.3 times may be measured with a colorimetric method as described herein.

In some other embodiments, a non-naturally occurring mutated arylsulfotransferase as disclosed herein does not comprise mutations in other positions that the ones above indicated.

In the description, the positions of the substituted amino acids are given with respect to the position of the amino acids of the rat arylsulfotransferase IV of amino acids sequence SEQ ID NO: 1.

The non-naturally occurring mutated arylsulfotransferases disclosed herein are isolated proteins.

The non-naturally occurring mutated arylsulfotransferases disclosed herein are recombinant proteins.

In some embodiments, the sequences of the non-naturally occurring mutated arylsulfotransferases may have at least 65%, 70%, 75%, 80%, 85%, 90%, 95%, or 99% identity over the entire sequence of SEQ ID NO:1.

In some embodiments, the sequences of the non-naturally occurring mutated arylsulfotransferases may have at least 75%, 80%, 85%, 90%, 95%, or 99% identity over the entire sequence of SEQ ID NO:1.

In some embodiments, the sequences of the non-naturally occurring mutated arylsulfotransferases may have at least 85%, 90%, 95%, or 99% identity over the entire sequence of SEQ ID NO:1.

In some embodiments, the sequences of the non-naturally occurring mutated arylsulfotransferases may have at least 90%, 95%, or 99% identity over the entire sequence of SEQ ID NO:1.

In some embodiments, the sequences of the non-naturally occurring mutated arylsulfotransferases may have at least 90% identity over the entire sequence of SEQ ID NO:1.

In some embodiments, the sequences of the non-naturally occurring mutated arylsulfotransferases may have at least 95% identity over the entire sequence of SEQ ID NO:1.

In some embodiments, the sequences of the non-naturally occurring mutated arylsulfotransferases may have at least 99% identity over the entire sequence of SEQ ID NO:1.

Homology or identity of sequence may be measured using known methods. For example, the UWGCG Package provides the BESTFIT program which can be used to calculate homology (for example used on its default settings) (Devereux et al (1984) Nucleic Acids Research 12, 387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10).

Software for performing BLAST analyses is publicly available through the National Center for Biotechnology Information (www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al, supra). These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Aced. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

While mutations are defined by reference to an amino acid position in the rat arylsulfotransferase IV of amino acids sequence SEQ ID NO: 1, equivalent substitutions at a homologous or corresponding position in the polypeptide chain of a homologue of arylsulfotransferase which shares at least 60%, or least 65%, 70%, 75%, 80%, 85%, 90%, 95%, or 99% amino acid identity with SEQ ID NO:1 are also encompassed. An equivalent position is determined by reference to the amino acid sequence SEQ ID NO: 1. The homologous or corresponding position can be readily deduced by lining up the sequence of the homologue and SEQ ID NO: 1 based on the homology between the sequences. The PILEUP and BLAST algorithms can be used to line up the sequences.

As examples of homologous sequences suitable for the present disclosure, one may cite the sequences of the arylsulfotransferase of Homo sapiens (SEQ ID NO: 2), Gallus gallus (SEQ ID NO: 3) or of Bos taurus (SEQ ID NO: 4).

In some embodiments, when a non-naturally occurring arylsulfotransferase is the rat arylsulfotransferase IV the substitutions are not F138A and/or Y236A. For example, when a non-naturally occurring arylsulfotransferase is the rat arylsulfotransferase IV and comprises one or two mutations, those are not F138A and/or Y236A.

In some embodiments, an enzyme mutant does not comprise any of the following substitutions: I239M, F138A, Y236A, the amino acid position being relative to the rat arylsulfotransferase IV of SEQ ID NO: 1.

A non-naturally occurring mutated arylsulfotransferase is not of any of the following sequences: SEQ ID NO: 1, SEQ ID NO: 2, SEQ ID NO: 3, or SEQ ID NO: 4.

The non-naturally occurring mutated arylsulfotransferases may comprise an amino acid substitution in any amino acid position or any combination of amino acid positions selected among positions 6, 7, 8, 9, 11, 17, 20, 33, 62, 97, 138, 195, 236, 239, 244, 263. They may comprise from only 1 to up to the 16 substitutions.

A non-naturally occurring mutated arylsulfotransferase may comprise an amino acid substitution in at least one amino acid position selected among positions 6, 7, 8, 9, 11, 17, 20, 33, 62, 97, 138, 195, 236, 239, 244, 263, and combinations thereof, and have an activity which is enhanced compared to the wild-type arylsulfotransferase of sequence SEQ ID NO: 1. An enhanced activity may be enhanced catalytic activity, or thermal stability or structure stability.

A non-naturally occurring mutated arylsulfotransferase may comprise an amino acid substitution in at least one amino acid position selected among positions 6, 7, 8, 9, 11, 17, 20, 33, 62, 97, 138, 195, 236, 239, 244, 263, and combinations thereof, and have an enhanced sulfotransferase catalytic activity compared to the wild-type arylsulfotransferase of sequence SEQ ID NO: 1.

Amino acid substitutions in anyone of positions 6, 7, 8, 9, 11, 33, 62, 97, 195, 263, and combinations thereof may advantageously impact the sulfotransferase catalytic activity of the mutants. Mutants with such mutations may have a sulfotransferase activity enhanced by at least 1.3 times compared to the wild-type arylsulfotransferase of sequence SEQ ID NO: 1.

Amino acid substitutions in anyone of positions 6, 7, 8, 9, 11, 33, 62, 97, 195, 263, and combinations thereof may have an enhanced stability, thermal and/or structural, compared to the wild-type arylsulfotransferase of sequence SEQ ID NO: 1. An amino acid substitution in anyone of those positions may advantageously impact thermal stability to the mutants. Amino acid substitutions in anyone of positions 33, 62, 97, 195, 263, and combinations thereof, for example in all the positions, may have an enhanced thermal stability. The thermal stability of the mutants may be higher than a wild-type arylsulfotransferase by at least about 1° C., about 2° C., about 3° C., about 4° C., about 5° C., about 6° C., about 10° C., about 15° C., about 20° C., or more, e.g., higher by about 1° C.-30° C., about 2° C.-25° C., about 3° C.-20° C., about 4° C.-15° C., about 5° C.-10° C., or about 6° C. The thermal stability of the mutants may be higher than a wild-type arylsulfotransferase by at least about 1° C. to about 6° C., or about 1° C., 2° C., 3° C., 4° C. 5° C., or about 6° C. As used herein, “thermal stability” refers to stability of a protein when exposed to higher temperature; a thermally stable mutant protein maintains its conformation at a higher temperature than a wild-type protein.

In other embodiments, amino acids substitutions may be in anyone of positions 17, 20, 138, 236, 239, 244, and combinations thereof. A substitution, alone or in combination, taken from this group of substituted positions may advantageously impact the sulfotransferase activity of the mutants. Mutants with such mutations may have a sulfotransferase activity enhanced by at least 1.3 times compared to the wild-type arylsulfotransferase of sequence SEQ ID NO: 1.

A non-naturally occurring mutated arylsulfotransferase may comprise at least 2, or at least 3, or at least 4, or at least 5, or at least 6, or at least 7, or at least 8, or at least 9, or 10 amino acid(s) substitution(s) in positions selected among positions 6, 7, 8, 9, 11, 33, 62, 97, 195, and 263.

In some embodiments, a non-naturally occurring mutated arylsulfotransferase may comprise no more than 2, or no more than 3, or no more than 4, or no more than 5, or no more than 6, or no more than 7, or no more than 8, or no more than 9 amino acid substitutions in positions selected among positions 6, 7, 8, 9, 11, 33, 62, 97, 195, and 263.

A non-naturally occurring mutated arylsulfotransferase may comprise at least 5 amino acid(s) substitution(s) in positions selected among positions 6, 7, 8, 9, 11, 33, 62, 97, 195, and 263.

A non-naturally occurring mutated arylsulfotransferase may comprise at least an amino acid substitution at least in position 6.

A non-naturally occurring mutated arylsulfotransferase may comprise at least an amino acid substitution in positions selected among positions 33, 62, 97, 195, and 263.

A non-naturally occurring mutated arylsulfotransferase may comprise at least an amino acid substitution in positions selected among positions 6, 33, 62, 97, 195, and 263.

In some embodiments, a non-naturally occurring mutated arylsulfotransferase may comprise an amino acid substitution at least in all the amino acid positions 6, 7, 8, 9, and 11. A non-naturally occurring mutated arylsulfotransferase comprising an amino acid substitution in all the positions 6, 7, 8, 9, and 11 further comprise an amino acid substitution in at least one, or in at least two, amino acid position(s) selected among positions 33, 62, 97, 195, and 263. A non-naturally occurring mutated arylsulfotransferase comprising an amino acid substitution in all the positions 6, 7, 8, 9, and 11 further comprise an amino acid substitution in at least one amino acid position(s) selected among positions 33, 62, 195, and 263, and does not comprise an amino acid substitution in position 97.

A non-naturally occurring mutated arylsulfotransferase may comprise an amino acid substitution at least in all the amino acid positions 6, 7, 8, 9, 11, and 33, and optionally an amino acid substitution in at least one amino acid position selected among positions 62, 97, 195, and 263.

A non-naturally occurring mutated arylsulfotransferase may comprise an amino acid substitution at least in all the amino acid positions 6, 7, 8, 9, 11, and 62, and optionally an amino acid substitution in at least one amino acid position selected among positions 33, 97, 195, and 263.

A non-naturally occurring mutated arylsulfotransferase may comprise at an amino acid substitution least in all the amino acid positions 6, 7, 8, 9, 11, and 97, and an amino acid substitution in at least one amino acid position selected among positions 33, 62, 195, and 263.

A non-naturally occurring mutated arylsulfotransferase may comprise an amino acid substitution at least in all the amino acid positions 6, 7, 8, 9, 11, and 195, and optionally an amino acid substitution in at least one amino acid position selected among positions 33, 62, 97, and 263.

A non-naturally occurring mutated arylsulfotransferase may comprise an amino acid substitution at least in all the amino acid positions 6, 7, 8, 9, 11, and 263, and optionally an amino acid substitution in at least one amino acid position selected among positions 33, 62, 97, and 195.

A non-naturally occurring mutated arylsulfotransferase comprising an amino acid substitution at least in all the positions 6, 7, 8, 9, and 11 may further comprise an amino acid substitution in at least one amino acid position selected among positions 33, 62, 195, and 263.

A non-naturally occurring mutated arylsulfotransferase comprising an amino acid substitution at least in all the amino acid positions 6, 7, 8, 9, and 11, may further comprise an amino acid substitution in at least one amino acid position selected among positions 33, 62, and 263.

A non-naturally occurring mutated arylsulfotransferase may comprise an amino acid substitution at least in all the amino acid positions 6, 7, 8, 9, and 11, and an amino acid substitution in at least one amino acid position selected among positions 33 and 62.

In some embodiment, a non-naturally occurring mutated arylsulfotransferase may comprise an amino acid substitution at least in all the amino acid positions 6, 7, 8, 9, and 11, does not comprise an amino acid substitution in position 97.

In some embodiment, a non-naturally occurring mutated arylsulfotransferase does not comprise an amino acid substitution in position 97.

In some embodiments, a non-naturally occurring mutated arylsulfotransferase may comprise an amino acid substitution at least in all the amino acid positions 33, 62, 97, 195, and 263. A non-naturally occurring mutated arylsulfotransferase comprising an amino acid substitution in all the amino acid positions 33, 62, 97, 195, and 263 may further comprise at least one amino acid substitution mutation in an amino acid position selected in the group of positions 6, 7, 8, 9, and 11.

A non-naturally occurring mutated arylsulfotransferase may comprise an amino acid substitution at least in all the amino acid positions 33, 62, 97, 195, 263, and 6, and optionally an amino acid substitution in at least one amino acid position selected among positions 7, 8, 9, and 11.

A non-naturally occurring mutated arylsulfotransferase may comprise an amino acid substitution at least in all the amino acid positions 33, 62, 97, 195, 263, and 7, and optionally an amino acid substitution in at least one amino acid position selected among positions 6, 8, 9, and 11.

A non-naturally occurring mutated arylsulfotransferase may comprise an amino acid substitution at least in all the amino acid position 33, 62, 97, 195, 263, and 8, and optionally an amino acid substitution at least in one amino acid position selected among positions 6, 7, 9, and 11.

A non-naturally occurring mutated arylsulfotransferase may comprise an amino acid substitution at least all the amino acid positions 33, 62, 97, 195, 263, and 9, and optionally an amino acid substitution in at least one amino acid position selected among positions 6, 7, 8, and 11.

A non-naturally occurring mutated arylsulfotransferase may comprise an amino acid substitution at least in all the amino acid positions 33, 62, 97, 195, 263, and 11, and optionally an amino acid substitution in at least one amino acid position selected among positions 6, 7, 8, and 9.

In some embodiments, a non-naturally occurring mutated arylsulfotransferase may comprise an amino acid substitution at least in all the amino acid positions 6, 33, 62, 97, 195, and 263.

In some embodiments, a non-naturally occurring mutated arylsulfotransferase may comprise an amino acid substitution at least in all the amino acid positions 6, 33, 62, 97, 195, 263, and 236 and optionally an amino acid substitution in at least one amino acid position selected among positions 7, 8, 9, and 11.

In some embodiments, a non-naturally occurring mutated arylsulfotransferase may comprise an amino acid substitution at least in all the amino acid positions 6, 33, 62, 195, 263, and 236 and optionally an amino acid substitution in at least one amino acid position selected among positions 7, 8, 9, and 11.

In some embodiments, a non-naturally occurring mutated arylsulfotransferase may comprise an amino acid substitution at least in all the amino acid positions 6, 33, 62, 195, 263, and 236 and optionally an amino acid substitution in at least one amino acid position selected among positions 7, 8, 9, and 11, and does not comprise an amino acid substitution in position 97.

In some embodiments, a non-naturally occurring mutated arylsulfotransferase may comprise an amino acid substitution at least in all the amino acid positions 6, 33, 62, 97, 263, and 236 and optionally an amino acid substitution in at least one amino acid position selected among positions 7, 8, 9, and 11, and does not comprise an amino acid substitution in position 195.

In some embodiments, a non-naturally occurring mutated arylsulfotransferase may comprise an amino acid substitution in the amino acid position 6, 7, 8, 9, 11, 33, 62, 97, and 263.

In some embodiments, a non-naturally occurring mutated arylsulfotransferase may comprise an amino acid substitution at least in all the amino acid position 6, 7, 8, 9, 11, 33, 62, 97, 195, and 263.

A non-naturally occurring mutated arylsulfotransferase may comprise an amino acid substitution in all the amino acid positions 6, 8, 9, 11, 33, 62, 97, 195, and 263, and optionally does not comprise an amino acid substitution in position 7.

A non-naturally occurring mutated arylsulfotransferase may comprise an amino acid substitution in all the amino acid positions 6, 7, 9, 11, 33, 62, 97, 195, and 263, and optionally does not comprise an amino acid substitution in position 8.

A non-naturally occurring mutated arylsulfotransferase may comprise an amino acid substitution in all the amino acid positions 6, 7, 8, 11, 33, 62, 97, 195, and 263, and optionally does not comprise an amino acid substitution in position 9.

A non-naturally occurring mutated arylsulfotransferase may comprise an amino acid substitution in all the amino acid positions 6, 7, 8, 9, 33, 62, 97, 195, and 263, and optionally does not comprise an amino acid substitution in position 11.

A non-naturally occurring mutated arylsulfotransferase may comprise an amino acid substitution in all the amino acid positions 6, 7, 8, 9, 11, 62, 97, 195, and 263, and optionally does not comprise an amino acid substitution in position 33.

A non-naturally occurring mutated arylsulfotransferase may comprise an amino acid substitution in all the amino acid positions 6, 7, 8, 9, 11, 33, 97, 195, and 263, and optionally does not comprise an amino acid substitution in position 62.

A non-naturally occurring mutated arylsulfotransferase may comprise an amino acid substitution in all the amino acid positions 6, 7, 8, 9, 11, 33, 62, 195, and 263, and optionally does not comprise an amino acid substitution in position 97.

A non-naturally occurring mutated arylsulfotransferase may comprise an amino acid substitution in all the amino acid positions 6, 7, 8, 9, 11, 33, 62, 97, and 263, and optionally does not comprise an amino acid substitution in position 195.

A non-naturally occurring mutated arylsulfotransferase may comprise an amino acid substitution in all the amino acid positions 6, 7, 8, 9, 11, 33, 62, 97, and 195, and optionally does not comprise an amino acid substitution in position 263.

A non-naturally occurring mutated arylsulfotransferase may comprise an amino acid substitution in all the amino acid positions 6, 7, 8, 9, 11, 33, 97, and 195, and optionally does not comprise an amino acid substitution in position 62 and/or 263.

A non-naturally occurring mutated arylsulfotransferase may comprise an amino acid substitution in all the amino acid positions 6, 7, 8, 9, 11, 33, and 97, and optionally does not comprise an amino acid substitution in position 62, 195 and/or 263.

In some embodiment, a non-naturally occurring mutated arylsulfotransferase does not comprise an amino acid substitution in position 195.

In some embodiment, a non-naturally occurring mutated arylsulfotransferase does not comprise an amino acid substitution in position 97.

In further embodiments, a non-naturally occurring mutated arylsulfotransferase may further comprise an amino acid substitution in at least 1, or at least 2, or at least 3, or at least 4, or at least 5, or at least 6 amino acid positions selected among positions 17, 20, 138, 236, 239, and 244. Alternatively, a non-naturally occurring mutated arylsulfotransferase may further comprise an amino acid substitution in no more than 1, or no more than 2, or no more than 3, or no more than 4, or no more than 5 amino acid positions selected among positions 17, 20, 138, 236, 239, and 244. In a further embodiment, a non-naturally occurring mutated arylsulfotransferase may comprise an amino acid substitution in at least one amino acid position selected among positions 17, 20, 138, 236, 239, and 244, independently of (or without) an amino acid substitution in amino acid positions 6, 7, 8, 9, 11, 33, 62, 97, 195, and 263.

In some embodiments, a non-naturally occurring mutated arylsulfotransferase may comprise an amino acid substitution at least in all the amino acid position 6, 7, 8, 9, 11, 33, 62, 97, 195, and 263, and possibly an amino acid substitution in at least one amino acid position selected among positions 17, 20, 138, 236, 239, and 244.

A non-naturally occurring mutated arylsulfotransferase may comprise an amino acid substitution at least in positions 6 and 236.

A non-naturally occurring mutated arylsulfotransferase may comprise an amino acid substitution at least in positions 6 and 236 and does not comprise an amino acid substitution in positions 97 and/or 195.

A non-naturally occurring mutated arylsulfotransferase may comprise an amino acid substitution at least in all the amino acid positions 6, 7, 8, 9, 11, 33, 62, 97, 195, 263, and j, and an amino acid substitution at least in one amino acid position selected among positions 20, 138, 236, 239, and 244.

A non-naturally occurring mutated arylsulfotransferase may comprise an amino acid substitution at least in all the amino acid positions 6, 7, 8, 9, 11, 33, 62, 97, 195, 263, and 20, and an amino acid substitution in at least one amino acid position selected positions 17, 138, 236, 239, and 244.

A non-naturally occurring mutated arylsulfotransferase may comprise an amino acid substitution at least in all the amino acid positions 6, 7, 8, 9, 11, 33, 62, 97, 195, 263, and 138, and an amino acid substitution in at least one amino acid position selected among positions 17, 20, 236, 239, and 244.

A non-naturally occurring mutated arylsulfotransferase may comprise an amino acid substitution at least in all the amino acid positions 6, 7, 8, 9, 11, 33, 62, 97, 195, 263, and 236, and optionally an amino acid substitution in at least one amino acid position selected among positions 17, 20, 138, 239, and 244.

A naturally occurring mutated arylsulfotransferase may comprise an amino acid substitution at least in all the amino acid positions 6, 7, 8, 9, 11, 33, 62, 97, 263 and 236, and optionally an amino acid substitution in at least one amino acid position selected among positions 17, 20, 138, 239, and 244.

A naturally occurring mutated arylsulfotransferase may comprise an amino acid substitution at least in all the amino acid positions 6, 7, 8, 9, 11, 33, 62, 97, 263 and 236, and optionally an amino acid substitution in at least one amino acid position selected among positions 17, 20, 138, 239, and 244, and does not comprise an amino acid substitution in position 195.

A naturally occurring mutated arylsulfotransferase may comprise an amino acid substitution at least in all the amino acid positions 6, 7, 8, 9, 11, 33, 62, 97, 263 and 236, and does not comprise an amino acid substitution in position 195.

A non-naturally occurring mutated arylsulfotransferase may comprise an amino acid substitution at least in all the amino acid positions 6, 7, 8, 9, 11, 33, 62, 97, 195, 263, and 239, and an amino acid substitution in at least one amino acid position selected among positions 17, 20, 138, 236, and 244.

A non-naturally occurring mutated arylsulfotransferase may comprise an amino acid substitution at least in all the amino acid positions 6, 7, 8, 9, 11, 33, 62, 97, 195, 263, and 244, and an amino acid substitution at least in one amino acid position selected among positions 17, 20, 138, 236, and 239.

In some embodiments, a substitution may be conservative, that is it replaces an amino acid with another amino acid of similar chemical structure, similar chemical properties or similar side-chain volume. The amino acids introduced may have similar polarity, hydrophilicity or hydrophobicity to the amino acids they replace. Conservative amino acid changes are well known in the art. Conservative amino acid changes may also be determined by reference to the Point Accepted Mutation (PAM) or BLOcks SUbstitution Matrix (BLOSUM) family of scoring matrices for conservation of amino acid sequence. Thus, conservative amino acid changes may be members of an equivalence group, being a set of amino acids having mutually positive scores in the similarity representation of the scoring matrix selected for use in an alignment of the reference and mutant polypeptide chains.

For example, a conservative substitution may be a substitution of an amino acid of one class by an amino acid of the same class:

TABLE 1
Classes of amino acids

| Class | Amino acids | 1-letter code |
|---|---|---|
| Aliphatic | Glycine, Alanine, Valine, Leucine, | G, A, V, L, |
| | Isoleucine | I |
| Hydroxyl | Serine, Cysteine, Selenocysteine, | S, C, U, T, |
| or sulfur/selenium- | Threonine, Methionine | M |
| containing | | |
| Cyclic | Proline | P |
| Aromatic | Phenylalanine, Tyrosine, Tryptophan | F, Y, W |
| Basic | Histidine, Lysine, Arginine | H, K, R |
| Acidic and their | Aspartate, Glutamate, Asparagine, | D, E, N, Q |
| amides | Glutamine | |

Alternatively, a conservative substitution may be a substitution of an amino acid of one class by an amino acid of another class but with a similar chemical structure, a similar chemical property and/or a similar side-chain volume.

Alternatively, in some embodiments, a substitution mutation may be a non-conservative mutation, which replaces the amino acid of one class with an amino acid of non-similar chemical structure, non-similar chemical property and/or non-similar side-chain volume.

As example, it is given a table of possible conservative mutations:

TABLE 2
Conservative amino acid substitutions

| Amino acid | |
|---|---|
| (1-letter code) | Conservative substitution |
| A | G, S, T, V |
| C | S, T, M |
| D | S, K, Q, H, N, E |
| E | P, D, S, R, K, Q, H, N |
| F | M, V, I, L, W, Y |
| G | A, S, N |
| H | D, E, N, M, R, Q |
| I | M, V, Y, F, L |
| K | D, E, N, Q, R |
| L | M, V, I, Y, F |
| M | H, Q, Y, F, L, I, V |
| N | G, D, E, T, S, R, K, Q, H |
| P | E, A, T, G |
| Q | D, E, N, H, M, S, R, K |
| R | E, N, H, Q, K |
| S | G, D, E, N, Q, A, T |
| T | N, S, V, A |
| V | T, A, M, F, L, I |
| W | F, Y |
| Y | H, M, I, L, F, W |

In some embodiments, a mutated arylsulfotransferase disclosed herein may comprise conservative and non-conservative substitutions.

A substituting amino acid in position 6 may be a glutamine (Q), or an asparagine (N). In some embodiments, a substituting amino acid in position 6 may be a glutamine (Q).

A substituting amino acid in position 7 may be an aspartate (D), or a glutamate (E). In some embodiments, a substituting amino acid in position 7 may be an aspartate (D).

A substituting amino acid in position 8 may be an alanine (A), a glycine (G), or a valine (V). In some embodiments, a substituting amino acid in position 8 may be an alanine (A).

A substituting amino acid in position 9 may be a glycine (G), an alanine (A) or a valine (V). In some embodiments, a substituting amino acid in position 9 may be a glycine (G).

A substituting amino acid in position 11 may be a leucine (L), a valine (V) or an isoleucine (1). In some embodiments, a substituting amino acid in position 11 may be a leucine (L).

A substituting amino acid in position 17 may be a phenylalanine (F) or a tyrosine (Y).

A substituting amino acid in position 20 may be an isoleucine (1) or a leucine (L).

A substituting amino acid in position 33 may be an arginine (R), an histidine (H) or a lysine (K). In some embodiments, a substituting amino acid in position 33 may be an arginine (R).

A substituting amino acid in position 62 may be an aspartate (D), or a glutamate (E). In some embodiments, a substituting amino acid in position 62 may be an aspartate (D).

A substituting amino acid in position 97 may be a serine (S), or a threonine (T). In some embodiments, a substituting amino acid in position 97 may be a serine (S).

A substituting amino acid in position 138 may be an histidine (H), a lysine (K) or an arginine (R). In some embodiments, a substituting amino acid in position 138 may be an histidine (H).

A substituting amino acid in position 195 may be an aspartate (D), or a glutamate (E). In some embodiments, a substituting amino acid in position 195 may be an aspartate (D).

A substituting amino acid in position 236 may be a phenylalanine (F), or a tryptophan (W). In some embodiments, a substituting amino acid in position 236 may be a phenylalanine (F).

A substituting amino acid in position 239 may be an aspartate (D), or a glutamate (E). In some embodiments, a substituting amino acid in position 239 may be an aspartate (D).

A substituting amino acid in position 244 may be an asparagine (N), or a glutamine (Q). In some embodiments, a substituting amino acid in position 244 may be an asparagine (N).

A substituting amino acid in position 263 may be an histidine (H), a lysine (K) or an arginine (R). In some embodiments, a substituting amino acid in position 263 may be an histidine (H).

In some embodiments, an amino acid substitution is not F138A and/or Y236A.

A non-naturally occurring mutated arylsulfotransferase may comprise at least one amino acid substitution selected in the group comprising P6Q, P7D, L8A, V9G, V11L, I17F, I17Y, F20L, F20I, W33R, K62D, A97S, F138H, N195D, Y236F, I239D, M244N, and T263H, and combination thereof.

A non-naturally occurring mutated arylsulfotransferase may comprise at least one amino acid substitution selected in the group comprising P6Q, P7D, L8A, V9G, V11L, W33R, K62D, A97S, N195D, T263H, and combination thereof.

A non-naturally occurring mutated arylsulfotransferase may comprise at least the amino acid substitution P6Q.

A non-naturally occurring mutated arylsulfotransferase may comprise all the amino acid substitutions P6Q, P7D, L8A, V9G, and V11L. A non-naturally occurring mutated arylsulfotransferase comprising all the amino acid substitutions P6Q, P7D, L8A, V9G, and V11L, may further comprise at least one, or in at least two, amino acid substitution(s) selected among W33R, K62D, A97S, N195D, and T263H. A non-naturally occurring mutated arylsulfotransferase comprising all the amino acid substitutions P6Q, P7D, L8A, V9G, and V11L, may further comprise at least one amino acid substitution selected among W33R, K62D, N195D, and T263H, and does not comprise the amino acid substitution A97S.

A non-naturally occurring mutated arylsulfotransferase may comprise all the amino acid substitutions P60, P7D, L8A, V9G, V11L, and W33R, and optionally at least one amino acid substitution selected among K62D, A97S, N195D, and T263H.

A non-naturally occurring mutated arylsulfotransferase may comprise all the amino acid substitutions P60, P7D, L8A, V9G, V11L, and K62D, and optionally at least one amino acid substitution selected among W33R, A97S, N195D, and T263H.

A non-naturally occurring mutated arylsulfotransferase may comprise all the amino acid substitutions P60, P7D, L8A, V9G, V11L, and A97S, and at least one amino acid substitution selected among W33R, K62D, N195D, and T263H.

A non-naturally occurring mutated arylsulfotransferase may comprise all the amino acid substitutions P60, P7D, L8A, V9G, V11L, and N195D, and optionally at least one amino acid substitution selected among W33R, K62D, A97S, and T263H.

A non-naturally occurring mutated arylsulfotransferase may comprise all the amino acid substitutions P60, P7D, L8A, V9G, V11L, and T263H, and optionally at least one amino acid substitution selected among W33R, K62D, A97S, and N195D.

A non-naturally occurring mutated arylsulfotransferase may comprise all the amino acid substitutions P60, P7D, L8A, V9G, V11L, and optionally at least one amino acid substitution selected among W33R, K62D, N195D, and T263H.

A non-naturally occurring mutated arylsulfotransferase may comprise all the amino acid substitutions P60, P7D, L8A, V9G, and V11L, may further comprise at least one amino acid substitution selected among W33R, K62D, and T263H.

A non-naturally occurring mutated arylsulfotransferase comprising all the amino acid substitutions P60, P7D, L8A, V9G, and V11L, may further comprise at least one amino acid substitution selected among W33R, and K62D.

In some embodiment, a non-naturally occurring mutated arylsulfotransferase comprising all the amino acid substitutions P60, P7D, L8A, V9G, and V11L, does not comprise the amino acid substitution A97S.

A non-naturally occurring mutated arylsulfotransferase may comprise all the amino acid substitutions W33R, K62D, A97S, N195D, and T263H. A non-naturally occurring mutated arylsulfotransferase comprising all the amino acid substitutions W33R, K62D, A97S, N195D, and T263H, may further comprise at least one amino acid substitution selected among P60, P7D, L8A, V9G, and V11L.

A non-naturally occurring mutated arylsulfotransferase may comprise at least all the amino acid substitutions W33R, K62D, A97S, N195D, T263H, and P60, and optionally at least one amino acid substitution selected among P7D, L8A, V9G, and V11L.

A non-naturally occurring mutated arylsulfotransferase may comprise at least all the amino acid substitutions W33R, K62D, A97S, N195D, T263H, and P7D, and optionally at least one amino acid substitution selected among P6Q, L8A, V9G, and V11L.

A non-naturally occurring mutated arylsulfotransferase may comprise at least all the amino acid substitutions W33R, K62D, A97S, N195D, T263H, and L8A, and optionally at least one amino acid substitution selected among P6Q, P7D, V9G, and V11L.

A non-naturally occurring mutated arylsulfotransferase may comprise at least all the amino acid substitutions W33R, K62D, A97S, N195D, T263H, and V9G, and optionally at least one amino acid substitution selected among P6Q, P7D, L8A, and V11L.

A non-naturally occurring mutated arylsulfotransferase may comprise at least all the amino acid substitutions W33R, K62D, A97S, N195D, T263H, and V11L, and optionally at least one amino acid substitution selected among P6Q, P7D, L8A, and V9G.

A non-naturally occurring mutated arylsulfotransferase may comprise the amino acid substitutions in all the positions W33R, K62D, A97S, N195D, and T263H, and optionally an amino acid substitution selected among P6Q and/or Y236F.

A non-naturally occurring mutated arylsulfotransferase may comprise the amino acid substitutions in all the positions P6Q, W33R, K62D, A97S, N195D, and T263H, and optionally at least one amino acid substitution selected among P7D, L8A, V9G, and V11L.

A non-naturally occurring mutated arylsulfotransferase may comprise the amino acid substitutions in all the positions P6Q, W33R, K62D, A97S, N195D, T263H, and Y236F, and optionally at least one amino acid substitution selected among P7D, L8A, V9G, and V11L.

A non-naturally occurring mutated arylsulfotransferase may comprise the amino acid substitutions in all the positions P6Q, W33R, K62D, N195D, T263H, and Y236F, and optionally at least one amino acid substitution selected among P7D, L8A, V9G, and V11L.

In some embodiment, a non-naturally occurring mutated arylsulfotransferase does not comprise the amino acid substitution A97S.

A non-naturally occurring mutated arylsulfotransferase may comprise the amino acid substitutions in all the positions P60, W33R, K62D, N195D, T263H, and Y236F, and optionally at least one amino acid substitution selected among P7D, L8A, V9G, and V11L, and does not comprise the amino acid substitution A97S.

In some embodiment, a non-naturally occurring mutated arylsulfotransferase does not comprise the amino acid substitution N195D.

A non-naturally occurring mutated arylsulfotransferase may comprise the amino acid substitutions in all the positions P60, W33R, K62D, T263H, and Y236F, and optionally at least one amino acid substitution selected among P7D, L8A, V9G, and V11L, and does not comprise the amino acid substitution N195D.

In some embodiments, a non-naturally occurring mutated arylsulfotransferase may comprise an amino acid substitution selected among P60, P7D, L8A, V9G, V11L, W33R, K62D, A97S, N195D, and T263H.

In some embodiments, a non-naturally occurring mutated arylsulfotransferase may comprise at least all the amino acid substitutions P60, P7D, L8A, V9G, V11L, W33R, K62D, A97S, N195D, and T263H.

A non-naturally occurring mutated arylsulfotransferase may comprise at least all the amino acid substitutions P60, L8A, V9G, V11L, W33R, K62D, A97S, N195D, and T263H, and optionally does not comprise the amino acid substitution P7D.

A non-naturally occurring mutated arylsulfotransferase may comprise at least all the amino acid substitutions P60, P7D, V9G, V11L, W33R, K62D, A97S, N195D, and T263H, and optionally does not comprise the amino acid substitution L8A.

A non-naturally occurring mutated arylsulfotransferase may comprise at least all the amino acid substitutions P60, P7D, L8A, V11L, W33R, K62D, A97S, N195D, and T263H, and optionally does not comprise the amino acid substitution V9G.

A non-naturally occurring mutated arylsulfotransferase may comprise at least all the amino acid substitutions P60, P7D, L8A, V9G, W33R, K62D, A97S, N195D, and T263H, and optionally does not comprise the amino acid substitution V11L.

A non-naturally occurring mutated arylsulfotransferase may comprise at least all the amino acid substitutions P6Q, P7D, L8A, V9G, V11L, K62D, A97S, N195D, and T263H, and optionally does not comprise the amino acid substitution W33R.

A non-naturally occurring mutated arylsulfotransferase may comprise at least all the amino acid substitutions P60, P7D, L8A, V9G, V11L, W33R, A97S, N195D, and T263H, and optionally does not comprise the amino acid substitution K62D.

A non-naturally occurring mutated arylsulfotransferase may comprise at least all the amino acid substitutions P60, P7D, L8A, V9G, V11L, W33R, K62D, N195D, and T263H, and optionally does not comprise the amino acid substitution A97S.

A non-naturally occurring mutated arylsulfotransferase may comprise at least all the amino acid substitutions P60, P7D, L8A, V9G, V11L, W33R, K62D, A97S, and T263H, and optionally does not comprise the amino acid substitution N195D.

A non-naturally occurring mutated arylsulfotransferase may comprise at least all the amino acid substitutions P60, P7D, L8A, V9G, V11L, W33R, K62D, A97S, and N195D, and optionally does not comprise the amino acid substitution T263H.

A non-naturally occurring mutated arylsulfotransferase may comprise at least all the amino acid substitutions P60, P7D, L8A, V9G, V11L, W33R, A97S, and N195D, and optionally does not comprise the amino acid substitution K62D and/or T263H.

A non-naturally occurring mutated arylsulfotransferase may comprise at least all the amino acid substitutions P60, P7D, L8A, V9G, V11L, W33R, and A97S, and optionally does not comprise the amino acid substitution K62D, N195D, and/or T263H.

In some embodiment, a non-naturally occurring mutated arylsulfotransferase does not comprise the amino acid substitution N195D.

In some embodiment, a non-naturally occurring mutated arylsulfotransferase does not comprise the amino acid substitution A97S.

In further embodiments, a non-naturally occurring mutated arylsulfotransferase may further comprise at least one, or at least 2, or at least 3, or at least 4, or at least 5, or at least 6 amino acid substitution(s) selected in the group comprising I17, F20, F138, Y236, I239, M244, and combinations thereof. Alternatively, a non-naturally occurring mutated arylsulfotransferase may further comprise an amino acid substitution in no more than 1, or no more than 2, or no more than 3, or no more than 4, or no more than 5 amino acid positions selected among I17, F20, F138, Y236, 239 and IM244. In a further embodiment, a non-naturally occurring mutated arylsulfotransferase may comprise an amino acid substitution in at least one amino acid position selected among I17, F20, F138, Y236, 239 and IM244, independently of (or without) an amino acid substitution selected among P60, P7D, L8A, V9G, V11L, W33R, K62D, A97S, N195D, and T263H.

In some embodiments, a non-naturally occurring mutated arylsulfotransferase may comprise at least all the amino acid substitutions P60, P7D, L8A, V9G, V11L, W33R, K62D, A97S, and T263H, and possibly at least one amino acid substitution selected in the group comprising I17F, I17Y, F20L, F20I, F138H, Y236F, I239D, M244N, and combinations thereof.

A non-naturally occurring mutated arylsulfotransferase may comprise at least the amino acid substitutions P60 and Y236F.

A non-naturally occurring mutated arylsulfotransferase may comprise at least the amino acid substitutions P60 and Y236F and does not comprise an amino acid substitution in positions 97 and/or 195.

A non-naturally occurring mutated arylsulfotransferase may comprise at least the amino acid substitutions P60 and Y236F and does not comprise the amino acid substitutions A97S and/or N195D.

A non-naturally occurring mutated arylsulfotransferase may comprise all the amino acid substitutions P60, P7D, L8A, V9G, V11L, W33R, K62D, A97S, N195D and T263H, and optionally Y236F.

A non-naturally occurring mutated arylsulfotransferase may comprise all the amino acid substitutions P60, L8A, V9G, V11L, W33R, K62D, A97S, N195D and T263H, and optionally Y236F, and optionally does not comprise the amino acid P7D.

A non-naturally occurring mutated arylsulfotransferase may comprise all the amino acid substitutions P60, P7D, V9G, V11L, W33R, K62D, A97S, N195D and T263H, and optionally Y236F, and optionally does not comprise the amino acid substitution L8A.

A non-naturally occurring mutated arylsulfotransferase may comprise all the amino acid substitutions P60, P7D, L8A, V11L, W33R, K62D, A97S, N195D and T263H, and optionally Y236F, and optionally does not comprise the amino acid substitution V9G.

A non-naturally occurring mutated arylsulfotransferase may comprise all the amino acid substitutions P60, P7D, L8A, V9G, W33R, K62D, A97S, N195D and T263H, and optionally Y236F, and optionally does not comprise the amino acid substitution V11L.

A non-naturally occurring mutated arylsulfotransferase may comprise all the amino acid substitutions P60, P7D, L8A, V9G, V11 L, K62D, A97S, N195D and T263H, and optionally Y236F, and optionally does not comprise the amino acid substitution W33R.

A non-naturally occurring mutated arylsulfotransferase may comprise all the amino acid substitutions P60, P7D, L8A, V9G, V11L, W33R, A97S, N195D and T263H, and optionally Y236F, and optionally does not comprise the amino acid substitution K62D.

A non-naturally occurring mutated arylsulfotransferase may comprise all the amino acid substitutions P60, P7D, L8A, V9G, V11L, W33R, K62D, N195D and T263H, and optionally Y236F, and optionally does not comprise the amino acid substitution A97S.

A non-naturally occurring mutated arylsulfotransferase may comprise all the amino acid substitutions P60, P7D, L8A, V9G, V11L, W33R, K62D, A97S, and T263H, and optionally Y236F, and optionally does not comprise the amino acid substitution N195D.

A non-naturally occurring mutated arylsulfotransferase may comprise all the amino acid substitutions P60, P7D, L8A, V9G, V11L, W33R, K62D, A97S and N195D, and optionally Y236F, and optionally does not comprise the amino acid substitution T263H.

A non-naturally occurring mutated arylsulfotransferase may comprise at least all the amino acid substitutions P60, P7D, L8A, V9G, V11L, W33R, K62D, A97S T263H, and Y236F, and optionally does not comprise the amino acid substitution N195D.

In some embodiments, a non-naturally occurring mutated arylsulfotransferase may comprise at least all the amino acid substitutions P60, P7D, L8A, V9G, V11L, W33R, K62D, A97S, N195D, T263H, and I17F, and at least one amino acid substitution selected in the group comprising F20L, F20I, F138H, Y236F, I239D, M244N, and combinations thereof.

In some embodiments, a non-naturally occurring mutated arylsulfotransferase may comprise at least all the amino acid substitutions P60, P7D, L8A, V9G, V11L, W33R, K62D, A97S, N195D, T263H, and I17Y, and at least one amino acid substitution selected in the group comprising F20L, F20I, F138H, Y236F, I239D, M244N, and combinations thereof.

In some embodiments, a non-naturally occurring mutated arylsulfotransferase may comprise at least all the amino acid substitutions P60, P7D, L8A, V9G, V11L, W33R, K62D, A97S, N195D, T263H, and F20L, and at least one amino acid substitution selected in the group comprising I17F, I17Y, F138H, Y236F, I239D, M244N, and combinations thereof.

In some embodiments, a non-naturally occurring mutated arylsulfotransferase may comprise at least all the amino acid substitutions P60, P7D, L8A, V9G, V11L, W33R, K62D, A97S, N195D, T263H, and F20I, and at least one amino acid substitution selected in the group comprising I17F, I17Y, F138H, Y236F, I239D, M244N, and combinations thereof.

In some embodiments, a non-naturally occurring mutated arylsulfotransferase may comprise at least all the amino acid substitutions P60, P7D, L8A, V9G, V11L, W33R, K62D, A97S, N195D, T263H, and F138H, and at least one amino acid substitution selected in the comprising I17F, I17Y, F20I, F20L, Y236F, I239D, M244N, and combinations thereof.

In some embodiments, a non-naturally occurring mutated arylsulfotransferase may comprise at least all the amino acid substitutions P60, P7D, L8A, V9G, V11L, W33R, K62D, A97S, N195D, T263H, and Y236F, and optionally at least one amino acid substitution selected in the group comprising I17F, I17Y, F20I, F20L, F138H, I239D, M244N, and combinations thereof.

In some embodiments, a non-naturally occurring mutated arylsulfotransferase may comprise at least all the amino acid substitutions P60, P7D, L8A, V9G, V11L, W33R, K62D, A97S, T263H, and Y236F, and optionally at least one amino acid substitution selected in the group comprising I17F, I17Y, F20I, F20L, F138H, I239D, M244N, and combinations thereof, and optionally does not comprise the amino acid substitution N195D.

In some embodiments, a non-naturally occurring mutated arylsulfotransferase may comprise at least all the amino acid substitutions P60, P7D, L8A, V9G, V11L, W33R, K62D, N195D, T263H, and Y236F, and optionally at least one amino acid substitution selected in the group comprising I17F, I17Y, F20I, F20L, F138H, I239D, M244N, and combinations thereof, and optionally does not comprise the amino acid substitution A97S.

In some embodiments, a non-naturally occurring mutated arylsulfotransferase may comprise at least all the amino acid substitutions P60, P7D, L8A, V9G, V11L, W33R, K62D, A97S, T263H, and Y236F, and does not comprise the amino acid substitution N195D.

In some embodiments, a non-naturally occurring mutated arylsulfotransferase may comprise at least all the amino acid substitutions P60, P7D, L8A, V9G, V11L, W33R, K62D, A97S, N195D, T263H, and I239D, and at least one amino acid substitution selected in the group comprising I17F, I17Y, F20I, F20L, F138H, Y236F, M244N, and combinations thereof.

In some embodiments, a non-naturally occurring mutated arylsulfotransferase may comprise at least all the amino acid substitutions P60, P7D, L8A, V9G, V11L, W33R, K62D, A97S, N195D, T263H, and M244N, and at least one amino acid substitution selected in the group comprising I17F, I17Y, F20I, F2L, F138H, Y236F, I239, and combinations thereof.

In some embodiments, a non-naturally occurring mutated arylsulfotransferase as disclosed herein may be a rat arylsulfotransferase IV of SEQ ID NO: 1 comprising the amino acid substitutions and combinations thereof as disclosed herein.

A non-naturally occurring arylsulfotransferase may comprise or may be an amino acids sequence as set forth in the following TABLE 3:

TABLE 3
Sequences of arylsulfotransferase mutants

| Mutants (Var) | SEQ ID NO: | SEQUENCE |
|---|---|---|
| Var01 | 5 | MEFSRPPLVHVKGIPLFKYFAETIGPLQNFTAWPDDLLISTYPK |
| I17F | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var04 | 6 | MEFSRPPLVHVKGIPLIKYLAETIGPLQNFTAWPDDLLISTYPK |
| F20L | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var03 | 7 | MEFSRPPLVHVKGIPLIKYIAETIGPLQNFTAWPDDLLISTYPK |
| F20I | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var05 | 8 | MEFSRPPLVHVKGIPLIKYFAETIGPLQNFTAWPDDLLISTYPK |
| F138H | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNHYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var06 | 9 | MEFSRPPLVHVKGIPLIKYFAETIGPLQNFTAWPDDLLISTYPK |
| Y236F | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNFTTIPTEIMDHNVSPFMRKGTTGDWKNTF |
| | | TVAQNERFDAHYAKTMTDCDFKFRCEL |
| Va07 | 10 | MEFSRPPLVHVKGIPLIKYFAETIGPLQNFTAWPDDLLISTYPK |
| M244N | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEINDHNVSPFMRKGTTGDWKNTF |
| | | TVAQNERFDAHYAKTMTDCDFKFRCEL |
| Var 02 | 11 | MEFSRPPLVHVKGIPLYKYFAETIGPLQNFTAWPDDLLISTYPK |
| I17Y | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var08 | 12 | MEFSRPPLVHVKGIPLIKYFAETIGPLQNFTAWPDDLLISTYPK |
| I239D | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTDPTEIMDHNVSPFMRKGTTGDWKNTF |
| | | TVAQNERFDAHYAKTMTDCDFKFRCEL |
| Var09 | 13 | MEFSRQDAGHLKGIPLIKYFAETIGPLQNFTARPDDLLISTYPK |
| P6Q | | SGTTWMSEILDMIYQGGDLEKCGRAPIYARVPFLEFKCPGVPSG |
| P7D | | LETLEETPSPRLLKTHLPLSLLPQSLLDQKVKVIYIARNAKDVV |
| L8A | | VSYYNFYNMAKLHPDPGTWDSFLENFMDGEVSYGSWYQHVKEWW |
| V9G | | ELRHTHPVLYLFYEDIKEDPKREIKKILEFLGRSLPEETVDSIV |
| V11L | | HHTSFKKMKENCMTNYTTIPTEIMDHNVSPFMRKGTTGDWKNHF |
| W33R | | TVAQNERFDAHYAKTMTDCDFKFRCEL |
| K62D | | |
| A97S | | |
| N195D | | |
| T263H | | |
| Var09-01 | 14 | MEFSRQPLVHVKGIPLIKYFAETIGPLQNFTAWPDDLLISTYPK |
| P6Q | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var09-02 | 15 | MEFSRPDLVHVKGIPLIKYFAETIGPLQNFTAWPDDLLISTYPK |
| P7D | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var09-03 | 16 | MEFSRPPAVHVKGIPLIKYFAETIGPLQNFTAWPDDLLISTYPK |
| L8A | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var09-04 | 17 | MEFSRPPLGHVKGIPLIKYFAETIGPLQNFTAWPDDLLISTYPK |
| V9G | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var09-05 | 18 | MEFSRPPLVHLKGIPLIKYFAETIGPLQNFTAWPDDLLISTYPK |
| V11L | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var09-06 | 19 | MEFSRPPLVHVKGIPLIKYFAETIGPLQNFTARPDDLLISTYPK |
| W33R | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var09-07 | 20 | MEFSRPPLVHVKGIPLIKYFAETIGPLQNFTAWPDDLLISTYPK |
| K62D | | SGTTWMSEILDMIYQGGDLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var09-08 | 21 | MEFSRPPLVHVKGIPLIKYFAETIGPLQNFTAWPDDLLISTYPK |
| A97S | | SGTTWMSEILDMIYQGGKLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var09-09 | 22 | MEFSRPPLVHVKGIPLIKYFAETIGPLQNFTAWPDDLLISTYPK |
| N195D | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var09-10 | 23 | MEFSRPPLVHVKGIPLIKYFAETIGPLQNFTAWPDDLLISTYPK |
| T263H | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 24 | MEFSRPDAGHLKGIPLIKYFAETIGPLQNFTARPDDLLISTYPK |
| -P6Q | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 25 | MEFSRQPAGHLKGIPLIKYFAETIGPLQNFTARPDDLLISTYPK |
| -P7D | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 26 | MEFSRQDLGHLKGIPLIKYFAETIGPLQNFTARPDDLLISTYPK |
| -L8A | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 27 | MEFSRQDAVHLKGIPLIKYFAETIGPLQNFTARPDDLLISTYPK |
| -V9G | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 28 | MEFSRQDAGHVKGIPLIKYFAETIGPLQNFTARPDDLLISTYPK |
| -V11L | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 29 | MEFSRQDAGHLKGIPLIKYFAETIGPLQNFTAWPDDLLISTYPK |
| -W33R | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 30 | MEFSRQDAGHLKGIPLIKYFAETIGPLQNFTARPDDLLISTYPK |
| -K62D | | SGTTWMSEILDMIYQGGKLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 31 | MEFSRQDAGHLKGIPLIKYFAETIGPLQNFTARPDDLLISTYPK |
| -A97S | | SGTTWMSEILDMIYQGGDLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 32 | MEFSRQDAGHLKGIPLIKYFAETIGPLQNFTARPDDLLISTYPK |
| -N195D | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 33 | MEFSRQDAGHLKGIPLIKYFAETIGPLQNFTARPDDLLISTYPK |
| -T263H | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| VAR09 | 34 | MEFSRQDAGHLKGIPLIKYFAETIGPLQNFTARPDDLLISTYPK |
| -K62D | | SGTTWMSEILDMIYQGGKLEKCGRAPIYARVPFLEFKCPGVPSG |
| -T263H | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| VAR09 | 35 | MEFSRQDAGHLKGIPLIKYFAETIGPLQNFTARPDDLLISTYPK |
| -K62D | | SGTTWMSEILDMIYQGGKLEKCGRAPIYARVPFLEFKCPGVPSG |
| -N195D | | LETLEETPSPRLLKTHLPLSLLPQSLLDQKVKVIYIARNAKDVV |
| -T263H | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var09 | 36 | MEFSRQDAGHLKGIPLFKYFAETIGPLQNFTARPDDLLISTYPK |
| +I17F | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 37 | MEFSRQDAGHLKGIPLYKYFAETIGPLQNFTARPDDLLISTYPK |
| +117Y | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 38 | MEFSRQDAGHLKGIPLIKYIAETIGPLQNFTARPDDLLISTYPK |
| +F20I | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 39 | MEFSRQDAGHLKGIPLIKYLAETIGPLQNFTARPDDLLISTYPK |
| +F20L | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 40 | MEFSRQDAGHLKGIPLIKYFAETIGPLQNFTARPDDLLISTYPK |
| +F138H | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNHYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var09 | 41 | MEFSRQDAGHLKGIPLIKYFAETIGPLQNFTARPDDLLISTYPK |
| +Y236F | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNFTTIPTEIMDHNVSPFMRKGTTGDWKNHF |
| | | TVAQNERFDAHYAKTMTDCDFKFRCEL |
| Var09 | 42 | MEFSRQDAGHLKGIPLIKYFAETIGPLQNFTARPDDLLISTYPK |
| +I239D | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTDPTEIMDHNVSPFMRKGTTGDWKNHF |
| | | TVAQNERFDAHYAKTMTDCDFKFRCEL |
| Var09 | 43 | MEFSRQDAGHLKGIPLIKYFAETIGPLQNFTARPDDLLISTYPK |
| +M244N | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEINDHNVSPFMRKGTTGDWKNHF |
| | | TVAQNERFDAHYAKTMTDCDFKFRCEL |
| Var5A | 44 | MEFSRQDAGHLKGIPLIKYFAETIGPLQNFTAWPDDLLISTYPK |
| P6Q | | SGTTWMSEILDMIYQGGKLEKCGRAPIYARVPFLEFKCPGVPSG |
| P7D | | LETLEETPAPRLLKTHLPLSLLPQSLLDQKVKVIYIARNAKDVV |
| L8A | | VSYYNFYNMAKLHPDPGTWDSFLENFMDGEVSYGSWYQHVKEWW |
| V9G | | ELRHTHPVLYLFYEDIKENPKREIKKILEFLGRSLPEETVDSIV |
| V11L | | HHTSFKKMKENCMTNYTTIPTEIMDHNVSPFMRKGTTGDWKNTF |
| | | TVAQNERFDAHYAKTMTDCDFKFRCEL |
| Var5B | 45 | MEFSRPPLVHVKGIPLIKYFAETIGPLQNFTARPDDLLISTYPK |
| W33R | | SGTTWMSEILDMIYQGGDLEKCGRAPIYARVPFLEFKCPGVPSG |
| K62D | | LETLEETPSPRLLKTHLPLSLLPQSLLDQKVKVIYIARNAKDVV |
| A97S | | VSYYNFYNMAKLHPDPGTWDSFLENFMDGEVSYGSWYQHVKEWW |
| N195D | | ELRHTHPVLYLFYEDIKEDPKREIKKILEFLGRSLPEETVDSIV |
| T263H | | HHTSFKKMKENCMTNYTTIPTEIMDHNVSPFMRKGTTGDWKNHF |
| | | TVAQNERFDAHYAKTMTDCDFKFRCEL |
| Var5A | 46 | MEFSRQDAGHLKGIPLIKYFAETIGPLQNFTARPDDLLISTYPK |
| +W33R | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var5A | 47 | MEFSRQDAGHLKGIPLIKYFAETIGPLQNFTAWPDDLLISTYPK |
| +K62D | | SGTTWMSEILDMIYQGGDLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var5A | 48 | MEFSRQDAGHLKGIPLIKYFAETIGPLQNFTAWPDDLLISTYPK |
| +A97S | | SGTTWMSEILDMIYQGGKLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var5A | 49 | MEFSRQDAGHLKGIPLIKYFAETIGPLQNFTAWPDDLLISTYPK |
| +N195D | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNTF |
| | | TVAQNERFDAHYAKTMTDCDFKFRCEL |
| Var5A | 50 | MEFSRQDAGHLKGIPLIKYFAETIGPLQNFTAWPDDLLISTYPK |
| +T263H | | SGTTWMSEILDMIYQGGKLEKCGRAPIYARVPFLEFKCPGVPSG |
| | | LETLEETPAPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKENPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var5B | 51 | MEFSRQPLVHVKGIPLIKYFAETIGPLQNFTARPDDLLISTYPK |
| +P6Q | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var5B | 52 | MEFSRPDLVHVKGIPLIKYFAETIGPLQNFTARPDDLLISTYPK |
| +P7D | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var5B | 53 | MEFSRPPAVHVKGIPLIKYFAETIGPLQNFTARPDDLLISTYPK |
| +L8A | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var5B | 54 | MEFSRPPLGHVKGIPLIKYFAETIGPLQNFTARPDDLLISTYPK |
| +V9G | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| Var5B | 55 | MEFSRPPLVHLKGIPLIKYFAETIGPLQNFTARPDDLLISTYPK |
| +V11L | | SGTTWMSEILDMIYQGGDLEKCGRAPIYARVPFLEFKCPGVPSG |
| | | LETLEETPSPRLLKTHLPLSLLPQSLLDQKVKVIYIARNAKDVV |
| | | VSYYNFYNMAKLHPDPGTWDSFLENFMDGEVSYGSWYQHVKEWW |
| | | ELRHTHPVLYLFYEDIKEDPKREIKKILEFLGRSLPEETVDSIV |
| | | HHTSFKKMKENCMTNYTTIPTEIMDHNVSPFMRKGTTGDWKNHF |
| | | TVAQNERFDAHYAKTMTDCDFKFRCEL |
| ″Var09- | 56 | MEFSRQDAGHLKGIPLIKYFAETIGPLQNFTARPDDLLISTYPK |
| N195D + Y236F″ | | SGTTWMSEILDMIYQGGDLEKCGRAPIYARVPFLEFKCPGVPSG |
| P6Q | | LETLEETPSPRLLKTHLPLSLLPQSLLDQKVKVIYIARNAKDVV |
| P7D | | VSYYNFYNMAKLHPDPGTWDSFLENFMDGEVSYGSWYQHVKEWW |
| L8A | | ELRHTHPVLYLFYEDIKENPKREIKKILEFLGRSLPEETVDSIV |
| V9G | | HHTSFKKMKENCMTNFTTIPTEIMDHNVSPFMRKGTTGDWKNHF |
| V11L | | TVAQNERFDAHYAKTMTDCDFKFRCEL |
| W33R | | |
| K62D | | |
| A97S | | |
| T263H | | |
| Y236F | | |

A non-naturally occurring mutated arylsulfotransferase may have an amino acid sequence selected among SEQ ID NO: 5 to 23, 25-35, 41, 45-47, and 49-56.

A non-naturally occurring mutated arylsulfotransferase may have an amino acids sequence having at least 60%, 65%, 70%, 75%, 80%, 85%, 90%, 95%, or 99% identity with a sequence selected among SEQ ID NO: 5 to 23, 25-35, 41, 45-47, and 49-56 and a sulfotransferase activity for converting adenosine 3′,5′-bisphosphate (PAP) into 3′-phosphoadenosine-5′-phosphosulfate (PAPS) at least about 1.3 times, or at least about twice, or at least about three times, greater than the said activity of the rat arylsulfotransferase IV of SEQ ID NO: 1.

A non-naturally occurring mutated arylsulfotransferase may have an amino acid sequence SEQ ID NO: 13.

A non-naturally occurring mutated arylsulfotransferase may have an amino acids sequence having at least 60%%, 65%, 70%, 75%, 80%, 85%, 90%, 95%, or 99% identity with a sequence SEQ ID NO: 13 (Var09) and a sulfotransferase activity for converting adenosine 3′,5′-bisphosphate (PAP) into 3′-phosphoadenosine-5′-phosphosulfate (PAPS) at least about 1.3 times or at least about twice, or at least about three times, greater than the said activity of the rat arylsulfotransferase IV of SEQ ID NO: 1.

A non-naturally occurring mutated arylsulfotransferase may have an amino acid sequence SEQ ID NO: 32.

A non-naturally occurring mutated arylsulfotransferase may have an amino acids sequence having at least 60%%, 65%, 70%, 75%, 80%, 85%, 90%, 95%, or 99% identity with a sequence SEQ ID NO: 32 (“Var09−N195D”) and a sulfotransferase activity for converting adenosine 3′,5′-bisphosphate (PAP) into 3′-phosphoadenosine-5′-phosphosulfate (PAPS) at least about 1.3 times or at least about twice, or at least about three times, greater than the said activity of the rat arylsulfotransferase IV of SEQ ID NO: 1.

A non-naturally occurring mutated arylsulfotransferase may have an amino acid sequence SEQ ID NO: 41.

A non-naturally occurring mutated arylsulfotransferase may have an amino acids sequence having at least 60%%, 65%, 70%, 75%, 80%, 85%, 90%, 95%, or 99% identity with a sequence SEQ ID NO: 41 (“Var09+Y236F”) and a sulfotransferase activity for converting adenosine 3′,5′-bisphosphate (PAP) into 3′-phosphoadenosine-5′-phosphosulfate (PAPS) at least about 1.3 times or at least about twice, or at least about three times, greater than the said activity of the rat arylsulfotransferase IV of SEQ ID NO: 1.

A non-naturally occurring mutated arylsulfotransferase may have an amino acid sequence SEQ ID NO: 45.

A non-naturally occurring mutated arylsulfotransferase may have an amino acids sequence having at least 60%%, 65%, 70%, 75%, 80%, 85%, 90%, 95%, or 99% identity with a sequence SEQ ID NO: 45 (“Var05B”) and a sulfotransferase activity for converting adenosine 3′,5′-bisphosphate (PAP) into 3′-phosphoadenosine-5′-phosphosulfate (PAPS) at least about 1.3 times or at least about twice, or at least about three times, greater than the said activity of the rat arylsulfotransferase IV of SEQ ID NO: 1.

A non-naturally occurring mutated arylsulfotransferase may have an amino acids sequence having at least 60%%, 65%, 70%, 75%, 80%, 85%, 90%, 95%, or 99% identity with a sequence SEQ ID NO: 45 (“Var05B”) and a sulfotransferase activity for converting adenosine 3′,5′-bisphosphate (PAP) into 3′-phosphoadenosine-5′-phosphosulfate (PAPS) substantially similar to the activity of the rat arylsulfotransferase IV of SEQ ID NO: 45.

A non-naturally occurring mutated arylsulfotransferase may have an amino acid sequence SEQ ID NO: 56.

A non-naturally occurring mutated arylsulfotransferase may have an amino acids sequence having at least 60%%, 65%, 70%, 75%, 80%, 85%, 90%, 95%, or 99% identity with a sequence SEQ ID NO: 56 (“Var09−N195D+Y236F”) and a sulfotransferase activity for converting adenosine 3′,5′-bisphosphate (PAP) into 3′-phosphoadenosine-5′-phosphosulfate (PAPS) at least about 1.3 times or at least about twice, or at least about three times, greater than the said activity of the rat arylsulfotransferase IV of SEQ ID NO: 1.

A non-naturally occurring mutated arylsulfotransferase is not an amino acids sequence selected among SEQ ID NO: 24, 36-40, 42-44 and 48.

The mutations disclosed herein may be introduced into the enzyme by using any methods known in the art, such as site directed mutagenesis of the enzyme, PCR and gene shuffling methods or by the use of multiple mutagenic oligonucleotides in cycles of site-directed mutagenesis. The mutations may be introduced in a directed or random manner. The mutagenesis method thus produces one or more polynucleotides encoding one or more different mutants. Typically, a library of mutant genes is produced which can be used to produce a library of mutant enzymes.

Recombinant Expression

The arylsulfotransferase mutants of the present disclosure can be produced by any suitable method, including recombinant and non-recombinant methods (e.g., chemical synthesis).

Where a non-naturally occurring mutated arylsulfotransferase is produced using recombinant techniques, the methods can involve any suitable construct and any suitable host cell, which can be a prokaryotic or eukaryotic cell, usually a bacterial or yeast host cell, more usually a bacterial cell. Methods for introduction of genetic material into host cells include, for example, transformation, electroporation, conjugation, calcium phosphate methods and the like. The method for transfer can be selected so as to provide for stable expression of the introduced non-naturally occurring arylsulfotransferase-encoding nucleic acid. The mutated arylsulfotransferase-encoding nucleic acid can be provided as an inheritable episomal element (e.g., plasmid) or can be genomically integrated.

The present disclosure provides nucleic acids, including isolated or recombinant nucleic acids, that comprise a nucleotide sequence encoding a non-naturally occurring mutated arylsulfotransferase as disclosed herein. In some embodiments, the present disclosure provides a nucleic acid (or nucleotide sequence) encoding a non-naturally occurring mutated arylsulfotransferase as disclosed herein. In some embodiments, the nucleotide sequence is operably linked to a transcriptional control element, e.g., a promoter. The promoter is in some cases constitutive. The promoter is in some cases inducible. In some cases, the promoter is suitable for use (e.g., active in) a prokaryotic host cell. In some cases, the promoter is suitable for use (e.g., active in) a eukaryotic host cell.

In some instances, a nucleic acid comprising a nucleotide sequence encoding a non-naturally occurring mutated arylsulfotransferase may be present in an expression vector. In some embodiments, the present disclosure provides a recombinant expression vector comprising a nucleic acid encoding a non-naturally occurring mutated arylsulfotransferase as disclosed herein. The present disclosure provides a recombinant expression vector (e.g., an isolated recombinant expression vector) that comprises a nucleotide sequence encoding a non-naturally occurring mutated arylsulfotransferase of the present disclosure.

In some embodiments, the nucleotide sequence encoding the mutated arylsulfotransferase is operably linked to a transcriptional control element, e.g., a promoter. The promoter is in some cases constitutive. The promoter is in some cases inducible. In some cases, the promoter is suitable for use (e.g., active in) a prokaryotic host cell. In some cases, the promoter is suitable for use (e.g., active in) a eukaryotic host cell.

Suitable vectors for transferring non-naturally occurring mutated arylsulfotransferase-encoding nucleic acid can vary in composition.

Integrative vectors can be conditionally replicative or suicide plasmids, bacteriophages, and the like. The constructs can include various elements, including for example, promoters, selectable genetic markers (e.g., genes conferring resistance to antibiotics (for instance kanamycin, erythromycin, chloramphenicol, or gentamycin)), origin of replication (to promote replication in a host cell, e.g., a bacterial host cell), and the like. The choice of vector will depend upon a variety of factors such as the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal. The choice of appropriate vector is well within the skill of the art. Many such vectors are available commercially.

In one example, the vector is an expression vector based on episomal plasmids containing selectable drug resistance markers and elements that provide for autonomous replication in different host cells (e.g., in both E. coli and N. meningitidis). One example of such a “shuttle vector” is the plasmid pFPIO (Pagotto et al. (2000) Gene 244: 13-19).

Constructs (recombinant vectors) can be prepared by, for example, inserting a polynucleotide of interest into a construct backbone, typically by means of DNA ligase attachment to a cleaved restriction enzyme site in the vector. Alternatively, the desired nucleotide sequence can be inserted by homologous recombination or site-specific recombination. Typically, homologous recombination is accomplished by attaching regions of homology to the vector on the flanks of the desired nucleotide sequence, while site-specific recombination can be accomplished through use of sequences that facilitate site-specific recombination (e.g., cre-lox, att sites, etc.). Nucleic acid containing such sequences can be added by, for example, ligation of oligonucleotides, or by polymerase chain reaction using primers comprising both the region of homology and a portion of the desired nucleotide sequence.

Vectors can provide for extrachromosomal maintenance in a host cell or can provide for integration into the host cell genome. Vectors are amply described in numerous publications well known to those in the art, including, e.g., Short Protocols in Molecular Biology, (1999) F. Ausubel, at al., eds., Wiley & Sons. Vectors may provide for expression of the nucleic acids encoding the protein of interest, may provide for propagating the subject nucleic acids, or both.

Examples of vectors that may be used include but are not limited to those derived from recombinant bacteriophage DNA, plasmid DNA or cosmid DNA. For example, plasmid vectors such as pBR322, pUC 19/18, pUC 118, 119 and the M13 mp series of vectors may be used. pET21 is also an expression vector that may be used. Bacteriophage vectors may include λgtl0, λgtl I, λgtl8-23, λZAP/R and the EMBL series of bacteriophage vectors. Further vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors.

For expression of a protein of interest, an expression cassette may be employed. Thus, the present disclosure provides a recombinant expression vector comprising a subject nucleic acid. The expression vector provides transcriptional and translational regulatory sequences, and may provide for inducible or constitutive expression, where the coding region is operably linked under the transcriptional control of the transcriptional initiation region, and a transcriptional and translational termination region. These control regions may be native to an arylsulfotransferase from which the non-naturally occurring mutated arylsulfotransferase is derived or may be derived from exogenous sources. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7, and the like).

Expression vectors generally have convenient restriction sites located near the promoter sequence to provide for the insertion of nucleic acid sequences encoding proteins of interest. A selectable marker operative in the expression host may be present to facilitate selection of cells containing the vector. In addition, the expression construct may include additional elements. For example, the expression vector may have one or two replication systems, thus allowing it to be maintained in organisms, for example in mammalian or insect cells for expression and in a prokaryotic host for cloning and amplification. In addition, the expression construct may contain a selectable marker gene to allow the selection of transformed host cells. Selection genes are well known in the art and will vary with the host cell used.

Isolation and purification of non-naturally occurring mutated arylsulfotransferases can be accomplished according to methods known in the art. For example, non-naturally occurring mutated arylsulfotransferases can be isolated from a lysate of cells genetically modified to express a non-naturally occurring mutated arylsulfotransferase, or from a synthetic reaction mix, by immunoaffinity purification, which generally involves contacting the sample with an anti-arylsulfotransferase antibody, washing to remove non-specifically bound material, and eluting specifically bound arylsulfotransferase. Isolated non-naturally occurring mutated arylsulfotransferase can be further purified by dialysis and other methods normally employed in protein purification methods. In one example, the non-naturally occurring mutated arylsulfotransferase can be isolated using metal chelate chromatography methods.

Any of a number of suitable host cells can be used in the production of non-naturally occurring mutated arylsulfotransferase. In general, the protein of interest described herein may be expressed in prokaryotes or eukaryotes, e.g., bacteria such as Escherichia coli in accordance with conventional techniques. Thus, the present disclosure further provides an in vitro host cell, which comprises a nucleic acid encoding a non-naturally occurring mutated arylsulfotransferase as disclosed herein. Host cells for production (including large scale production) of a protein of interest can be selected from any of a variety of available host cells.

Examples of host cells for expression include those of a prokaryotic or eukaryotic unicellular organism, such as bacteria (e.g., Escherichia coli strains), yeast (e.g., Saccharomyces cerevisiae, Pichia spp., and the like), and may include host cells originally derived from a higher organism such as insects, vertebrates, e.g., mammals. Suitable bacteria include but are not limited to BL21 Competent E. coli, BL21(DE3) Competent E. coli, NEB Express Competent E. col, NEB Express Iq Competent E. coli, T7 Express Competent E. coli, T7 Express Iq Competent E. coli, T7 Express lysY Competent E. coli, T7 Express lysY/lq Competent E. coli, T7 Express Crystal Competent E. coli, SHuffle Express Competent E. coli, SHuffle T7 Express Competent E. coli, SHuffle T7 Express lysY Competent E. coli, SHuffle T7 Competent E. coli, NiCo21(DE3) Competent E. coli, Lemo21(DE3) Competent E. coli. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, and the like). In some cases, bacterial host cells and yeast host cells are of particular interest for production of the protein of interest.

Non-naturally occurring mutated arylsulfotransferases can be prepared in substantially pure or substantially isolated form. Purified non-naturally occurring mutated arylsulfotransferases can be provided such that the polypeptide is present in a composition that is substantially free of other expressed polypeptides, e.g., less than 90%, usually less than 60% and more usually less than 50% of the composition is made up of other expressed polypeptides.

Kits

In some embodiments, the disclosure relates to a kit for sulfating a substrate.

A kit for sulfating a substrate may comprise at least:

one non-naturally occurring mutated arylsulfotransferase as disclosed herein in a first container; and

a sulfo group donor in a second container.

A kit as disclosed herein may be used for sulfating a polysaccharide. A kit as disclosed herein may be used for converting adenosine 3′,5′-bisphosphate (PAP) into 3′-phosphoadenosine-5′-phosphosulfate (PAPS). A kit as disclosed herein may be used for synthesizing a sulfated substrate. A kit as disclosed herein may be used for synthesizing an heparan sulfate. A kit as disclosed herein may be used for synthesizing a sulfated heparin.

A sulfo donor group may be an aryl sulfate compound.

An aryl sulfate compound may be pNPS.

The kit may further comprise instructions for sulfating a substrate, for example a polysaccharide. The instructions may concern the synthesis of heparin.

A kit may further contain a buffer suitable for the catalytic activity of the enzyme. The buffer may be packaged with an arylsulfotransferase as disclosed herein or may be packaged in a separate container. A suitable buffer may be, for example, TRIS-buffer, sodium phosphate buffer, and potassium phosphate buffer. A suitable pH is about 6.0 to about 7.5, and about 7.0.

In some embodiments, the kit may comprise at least one further enzyme. An additional enzyme may be a glycosyltransferase, an N-deacetylase/N-sulfotransferase, a C5-epimerase, or an O-sulfotransferase (OST) enzyme, such as for example 2-OST, 3-OST, 3-OST-1, 3-OST-3, 6-OST, 6-OST-1, 6-OST3. When a kit contains two or more enzyme, each enzyme may be packaged in separate container.

Arylsulfotransferase Mutant Catalytic Activity and Screening Methods

A non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least about 1.3 times greater than the said activity of the rat arylsulfotransferase IV of SEQ ID NO: 1.

An arylsulfotransferase activity may be detected and measured according to any known method in the art.

In some embodiments, the increase of activity of non-naturally occurring mutated arylsulfotransferase of at least about 1.3 times compared with the activity of the rat arylsulfotransferase IV of SEQ ID NO: 1 may be measured with a colorimetric method allowing measuring the amount of p-Nitrophenyl (pNP) released (or produced) by the transfer of the sulfuryl group from p-Nitrophenyl sulfate (pNPS) to 3′,5′-adenosine-phosphate (PAP) for the production of 3′-phosphoadenosine-5′-phosphosulfate (PAPS) according to the following scheme reaction:

PAP+pNPS→PAPS+pNP

The method may comprise the steps of:

a) contacting a non-naturally occurring mutated arylsulfotransferase, for example expressed in bacteria or provided in a lysate of bacteria expressing said non-naturally occurring mutated arylsulfotransferase, or provided in a purified form, with a sufficient amount of pNPS and PAP, in a suitable buffer,

b) acquiring a measure representative of pNP produced at step a),

c) contacting a rat arylsulfotransferase IV of SEQ ID NO: 1, for example expressed in bacteria or provided in a lysate of bacteria expressing said non-naturally occurring mutated arylsulfotransferase, or provided in a purified form, with a sufficient amount of pNPS and PAP, in a suitable buffer,

d) acquiring a measure representative of pNP produced at step c), and

e) comparing the measures obtained at step b) and at step d).

Bacteria suitable for the expression of a mutated or a wild-type arylsulfotransferase (such as the rat arylsulfotransferase IV of SEQ ID NO: 1) may be E. coli BL21 DE3. The amount of enzyme suitable for the reaction, whatever the manner it is provided, may be of about 30 ng/μL.

Sufficient amounts of pNPS and PAP, when 30 ng/μL of enzyme are used, may be, respectively of about 1 mM and of about 0.23 mM.

A measure representative of pNP produced during the reaction may be obtained by a measure of the optical density at 404 nm, for example using a SpectraMax® 190 from Molecular Devices according to manufacturer's recommendations. The obtained measure may be expressed in arbitrary Unit of absorbance.

A suitable buffer for the reaction may be a phosphate buffer at pH 7.0 comprising glycerol at 10%.

A suitable temperature of reaction may be about 37° C.

The acquisition of the measure may be carried out 10, 30 or 90 minutes after initiation of the reaction, for example 10 minutes after initiation of the reaction.

In some embodiments, a blank may be subtracted to normalize the acquired measures. A blank may be water or a buffer without enzyme and substrates.

Alternatively, in some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of at least one of the mutated arylsulfotransferase of SEQ ID NO: 5 to 23, 25-35, 41, 45-47, and 49-56.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of anyone of the mutated arylsulfotransferases of SEQ ID NO: 5 to 23, 25-35, 41, 45-47, and 49-56.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 5.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 6.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 7.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 8.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 9.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 10.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 11.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 12.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 13.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 14.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 15.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 16.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 17.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 18.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 19.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 20.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 21.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 22.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 23.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 25.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 26.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 27.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 28.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 29.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 30.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 31.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 32.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 33.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 34.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 35.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 41.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 45.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 46.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 47.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 49.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 50.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 51.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 52.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 53.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 54.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 55.

In some embodiments, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of the mutated arylsulfotransferase of SEQ ID NO: 56.

The arylsulfotransferase mutants of the present disclosure display an enhanced sulfation activity with respect to the corresponding wild-type rat AST IV enzyme. Enhanced sulfation activity may be characterized in terms of an increased catalytic efficiency or an increased product formation rate with one or more substrates for sulfation. The increased coupling efficiency or increased product formation rate may or may not be shared across all substrates utilized by the arylsulfotransferase mutants of the present disclosure. In some embodiments, the enhanced catalytic activity of the mutated arylsulfotransferases disclosed herein, compared to the wild-type rat AST IV enzyme, is the reverse reaction generating PAPS from PAP with a sulfo donor group, such as pNPS.

The enzymatic activity of an arylsulfotransferase as disclosed herein may be measured in vitro using any of the substrates or conditions suitable for giving a sulfation rate, a metabolite formation rate, or substrate rate disappearance. For example, an arylsulfotransferase activity may be detected and measured with a colorimetric method. In such method, a colorimetric enzyme substrate or colorimetric metabolite may be used. The disappearance of the colorimetric substrate may be detected and measured, and/or the appearance of the colorimetric metabolite may be detected and measured.

A transformation rate may be measured.

The substrate for the sulfation process may be any organic compound capable of being sulfated by an arylsulfotransferase enzyme. The suitability of any organic compound for sulfation by an arylsulfotransferase enzyme may be routinely determined by the methods described herein.

The substrate can either be a natural substrate of a wild-type arylsulfotransferase enzyme or a substrate which is not normally a substrate for the wild-type enzyme, but which is capable of being utilized as such in the mutant enzyme. Examples of natural substrates for the arylsulfotransferase enzymes are 3′,5′-adenosine-phosphate (PAP) or p-nitrophenyl sulfate (pNPS).

For detecting and measuring an arylsulfotransferase activity on the conversion of 3′,5′-adenosine-phosphate (PAP) into 3′-phosphoadenosine-5′-phosphosulfate (PAPS), one may use as sulfo donor group the p-nitrophenyl sulfate (pNPS) which is converted in a colorimetric metabolite the p-nitrophenyl (pNP), according to the following scheme:

PAP+pNPS→PAPS+pNP

The presence of pNP may be detected and measured by absorbance detection measured at 404 nm.

Suitable parameters for detecting and measuring an arylsulfotransferase activity in such method may be using about 30 ng/μL of enzyme, pNPS at 1 mM, PAP at 0.23 mM, in phosphate buffer at pH 7.0; with glycerol at 10%. The reaction mixture may be incubated at about 37° C., for 10, 30 or 90 minutes. The reaction is initiated by addition of PAP to a mixture of enzyme and pNPS.

In some embodiments, an arylsulfotransferase activity may be detected and measured according to the reaction: PAP+pNPS→PAPS+pNP, by measuring the amount of the formed metabolite pNP.

In some embodiments, the sulfotransferase activity for converting adenosine 3′,5′-bisphosphate (PAP) into 3′-phosphoadenosine-5′-phosphosulfate (PAPS) may be at least about 1.5 times, or at least about 1.8, or at least about 1.9, or at least about 2.0, or at least about 2.2, or at least about 2.5, or at least about 3.0, or at least about 3.2, or at least about 3.5, or at least about 4.0, or at least about 4.5, or at least about 5.0, or at least about 5.5, or at least about 6.0, or at least about 6.5, or at least about 7.0 times greater than the said activity of the rat arylsulfotransferase IV of SEQ ID NO: 1. The increase of activity of a non-naturally occurring mutated arylsulfotransferase compared with the activity of the rat arylsulfotransferase IV of SEQ ID NO: 1 may be measured with a colorimetric method as described herein.

The sulfotransferase activity for converting adenosine 3′,5′-bisphosphate (PAP) into 3′-phosphoadenosine-5′-phosphosulfate (PAPS) may be measured by detection of a metabolite resulting from the transformation of a sulfo donor group, e.g., the detection of pNP resulting from the transformation of the pNPS. In such case, an enhanced transformation rate of pNPS into pNP in the reaction PAP+pNPS→PAPS+pNP is tantamount to an enhanced transformation rate PAP into PAPS.

As above indicated the non-naturally occurring arylsulfotransferase may be used isolated and purified or within a recombinant host cell, such as a recombinant E. coli. Bacteria suitable for the expression of a mutated or a wild-type arylsulfotransferase (such as the rat arylsulfotransferase IV of SEQ ID NO: 1) may be E. coli BL21 DE3. The amount of enzyme suitable for the reaction, whatever the manner it is provided, may be of about 30 ng/μL.

Sufficient amounts of pNPS and PAP, when 30 ng/μL of enzyme are used, may be, respectively of about 1 mM and of about 0.23 mM.

For the catalytic enzyme reaction to occur, the reaction medium may be placed at a temperature ranging from about 20° C. to about 40° C., or from about 25° C. to about 37° C., or from about 30° C. to about 35° C. For example, a temperature of reaction may be at about 37° C. As other example, a temperature of reaction may be at about 40° C.

The optical density (OD) or absorbance may be read at different period of time after the initiation of the catalytic reaction, for example 0, 10, 30 or 90 minutes to measure a rate of catalytic activity. A blank may be subtracted to normalize the acquired measures. A blank may be water or a buffer without enzyme and substrates

The mutations disclosed herein may be introduced into the enzyme by using any methods known in the art, such as site directed mutagenesis of the enzyme, PCR and gene shuffling methods or by the use of multiple mutagenic oligonucleotides in cycles of site-directed mutagenesis. The mutations may be introduced in a directed or random manner. The mutagenesis method thus produces one or more polynucleotides encoding one or more different mutants. Typically, a library of mutant genes is produced which can be used to produce a library of mutant enzymes, which thereafter may be screened according to the methods disclosed hereafter. Alternatively, the nucleic acids encoding the mutated enzyme disclosed herein may be obtained using any gene synthesis methods known in the art.

The arylsulfotransferase mutants may be screened either after extraction and purification from the recombinant cells or within the recombinant cells used to produce them.

A screening method may use the conversion of 3′,5′-adenosine-phosphate (PAP) into 3′-phosphoadenosine-5′-phosphosulfate (PAPS) in presence of p-nitrophenyl sulfate (pNPS) as sulfo donor group. The measure of the p-nitrophenyl (pNP) metabolite formation may be used to search for enhanced catalytic activity.

An enhanced catalytic activity of at least 1.3-fold compared to the catalytic activity of the wild-type enzyme may be used as reference threshold to identify mutated arylsulfotransferase with enhanced catalytic activity of interest.

Alternatively, a catalytic activity corresponding to any one of the arylsulfotransferases disclosed herein, and for example having an amino acids sequence selected in the group comprising SEQ ID NO: 5 to 23, 25-35, 41, 45-47, and 49-56, may be used as reference threshold to identify mutated arylsulfotransferase with enhanced catalytic activity of interest.

In some embodiments, a method of screening and/or selecting a non-naturally occurring mutated arylsulfotransferase comprising at least one amino acid substitution mutation and comprising a sulfotransferase activity for converting adenosine 3′,5′-bisphosphate (PAP) into 3′-phosphoadenosine-5′-phosphosulfate (PAPS) being at least 1.3 times greater than the said activity of the rat arylsulfotransferase IV of SEQ ID NO: 1 or being at least substantially the same or greater than said activity of a non-naturally occurring mutated arylsulfotransferase having an amino acids sequence selected in the group comprising SEQ ID NO: 5 to 23, 25-35, 41, 45-47, and 49-56, may comprise at least the steps of:

a) contacting a non-naturally occurring mutated arylsulfotransferase candidate comprising at least one amino acid substitution mutation with a sulfo group donor in conditions suitable for a transfer of the sulfo group from the sulfo group donor to PAP to obtain PAPS,

b) detecting a rate or an amount of formation of PAPS,

c) comparing the rate or amount of formation of PAPS obtained at step b) with a rate or an amount of reference obtained with a rat arylsulfotransferase IV of SEQ ID NO: 1 or obtained with a non-naturally occurring mutated arylsulfotransferase having an amino acids sequence selected in the group comprising SEQ ID NO: 5 to 23, 25-35, 41, 45-47, and 49-56, and

d) selecting any non-naturally occurring mutated arylsulfotransferase candidate comprising at least one amino acid substitution mutation and comprising a sulfotransferase activity for converting adenosine 3′,5′-bisphosphate (PAP) into 3′-phosphoadenosine-5′-phosphosulfate (PAPS) being at least 1.3 times greater than said activity of the rat arylsulfotransferase IV of SEQ ID NO: 1 or being at least substantially the same or greater than said activity of a non-naturally occurring mutated arylsulfotransferase having an amino acids sequence selected in the group comprising SEQ ID NO: 5 to 23, 25-35, 41, 45-47, and 49-56.

The increase of activity of a non-naturally occurring mutated arylsulfotransferase compared with the activity of the rat arylsulfotransferase IV of SEQ ID NO: 1 may be measured with a colorimetric method as described herein.

The detection of a rate or an amount of formation of PAPS at step b) may be carried out directly by measuring the amount of metabolite PAPS, indirectly by measuring the amount of the product to be transformed PAP.

Alternatively, the detection of a rate or an amount of formation of PAPS at step b) may be carried out by measuring the amount of a sulfo donor group, e.g., pNPS, or by measuring the amount of the metabolite of the sulfo donor group, e.g., pNP.

In one embodiment, the method disclosed herein may implement a sulfo group donor such as p-nitrophenyl sulfate.

In one embodiment, the method disclosed herein implements pNPS as a sulfo donor group, and step b) of detecting a rate or an amount of formation of PAPS is indirectly carried out by detecting a rate or an amount of formation of pNP from pNPS.

In some embodiments, the present disclosure also relates to a non-naturally occurring mutated arylsulfotransferase comprising at least one amino acid substitution mutation and comprising a sulfotransferase activity for converting adenosine 3′,5′-bisphosphate (PAP) into 3′-phosphoadenosine-5′-phosphosulfate (PAPS) being at least 1.3 times greater than the said activity of the rat arylsulfotransferase IV of SEQ ID NO: 1 or being at least the same than said activity of a non-naturally occurring mutated arylsulfotransferase having an amino acids sequence selected in the group comprising SEQ ID NO: 5 to 23, 25-35, 41, 45-47, and 49-56 identified by a method as disclosed herein.

Uses and Methods for Sulfating a Substrate

Sulfation

In some embodiments the disclosure, relates to a use of a non-naturally occurring mutated arylsulfotransferase as disclosed herein for sulfating a substrate.

In some embodiments the disclosure, relates to a method of sulfating a substrate comprising at least a step of contacting the substrate to be sulfated with a) a non-naturally occurring mutated arylsulfotransferase as disclosed herein and b) a sulfo group donor in conditions suitable for a transfer of the sulfo group from the sulfo group donor to said substrate.

The uses or methods of the disclosure may be for converting adenosine 3′,5′-bisphosphate (PAP) into 3′-phosphoadenosine-5′-phosphosulfate (PAPS).

The uses or methods of the disclosure may be synthesizing heparin.

A non-naturally occurring mutated arylsulfotransferase as disclosed herein comprises an amino acid substitution in at least one amino acid position selected among 6, 7, 8, 9, 11, 17, 20, 33, 62, 97, 138, 195, 236, 239, 244, 263, and combinations thereof, wherein the amino acid position is relative to the amino acids sequence of rat arylsulfotransferase IV SEQ ID NO: 1, and comprises an amino acid sequence having at least 60% sequence identity with amino acids sequence SEQ ID NO: 1, with the proviso that when said arylsulfotransferase is the rat arylsulfotransferase IV, the mutations are not F138A and/or Y236A.

A non-naturally occurring mutated arylsulfotransferase has a sulfotransferase activity for converting adenosine 3′,5′-bisphosphate (PAP) into 3′-phosphoadenosine-5′-phosphosulfate (PAPS) at least about 1.3 times greater than the said activity of the rat arylsulfotransferase IV of SEQ ID NO: 1.

A non-naturally occurring mutated arylsulfotransferase comprises an amino acid substitution in at least one amino acid position selected among positions 6, 7, 8, 9, 11, 17, 20, 33, 62, 97, 138, 195, 236, 239, 244, 263, and combinations thereof, wherein the amino acid position is relative to the amino acids sequence of rat arylsulfotransferase IV SEQ ID NO: 1, and comprises an amino acid sequence having at least 60% sequence identity with amino acids sequence SEQ ID NO: 1, and wherein said non-naturally occurring mutated arylsulfotransferase has a sulfotransferase activity for converting adenosine 3′,5′-bisphosphate (PAP) into 3′-phosphoadenosine-5′-phosphosulfate (PAPS) at least about 1.3 times greater than the said activity of the rat arylsulfotransferase IV of SEQ ID NO: 1.

Alternatively, in some embodiments, in the uses and methods disclosed herein, a non-naturally occurring mutated arylsulfotransferase as disclosed herein may have a sulfotransferase activity for converting adenosine 3′,5′-bisphosphate (PAP) into 3′-phosphoadenosine-5′-phosphosulfate (PAPS) at least substantially similar to or greater than the catalytic activity of at least one of the mutated arylsulfotransferase of SEQ ID NO: 5 to 23, 25-35, 41, 45-47, and 49-56.

A method for sulfating a substrate as disclosed herein may comprise at least a step of contacting said substrate to be sulfated with:

a) a non-naturally occurring mutated arylsulfotransferase as disclosed herein, and

b) a sulfo group donor in conditions suitable for a transfer of the sulfo group from the sulfo group donor to said substrate.

The method may further comprise a step of retrieving the sulfated substrate.

A substrate to be sulfated may be selected in a group comprising adenosine 3′,5′-bisphosphate (PAP), a polysaccharide, an heparan, an heparosan sulfate, or a sulfated heparin.

The disclosure relates to a method for obtaining a sulfated substrate by sulfating a substrate with at least one sulfotransferase and PAPS, said method including at least one step of converting PAP into PAPS by contacting said PAP with a non-naturally occurring mutated arylsulfotransferase comprising (1) an amino acid substitution in at least one amino acid position selected among positions 6, 7, 8, 9, 11, 17, 20, 33, 62, 97, 138, 195, 236, 239, 244, 263, and combinations thereof, wherein the amino acid position is relative to the amino acids sequence of rat arylsulfotransferase IV SEQ ID NO: 1, and (2) an amino acid sequence having at least 60% amino acid sequence identity to SEQ ID NO: 1.

According to a specific embodiment, the step of converting PAP into PAPS is simultaneous to the step of sulfation.

According to another embodiment, the step of converting PAP into PAPS and the step of sulfation are sequential.

The disclosure relates to a method for sulfating a substrate with a sulfotransferase and PAPS in conditions suitable to transfer a sulfo group from PAPS to the substrate to be sulfated and to obtain a sulfated substrate and PAP, comprising at least a step of converting the PAP so-obtained into PAPS by contacting the PAP with:

(i) a non-naturally occurring mutated arylsulfotransferase comprising (1) at least one amino acid substitution mutation in an amino acid position selected in the group of positions 6, 7, 8, 9, 11, 17, 20, 33, 62, 97, 138, 195, 236, 239, 244, 263, and combination thereof, wherein the amino acid position is relative to the rat arylsulfotransferase IV of SEQ ID NO: 1, and (2) an amino acid sequence having at least 60% amino acid sequence identity to SEQ ID NO: 1, and

(ii) a sulfo group donor

in conditions suitable for a transfer of the sulfo group from the sulfo group donor to PAP to obtain PAPS

The disclosure relates to a method for sulfating a substrate, comprising at least the steps of:

a) sulfating a substrate with a sulfotransferase and PAPS in conditions suitable to transfer a sulfo group from PAPS to the substrate to be sulfated and to obtain a sulfated substrate and PAP, and

b) converting the PAP obtained at step a) into PAPS by contacting the PAP with

(i) a non-naturally occurring mutated arylsulfotransferase comprising (1) an amino acid substitution in at least one amino acid position selected among positions 6, 7, 8, 9, 11, 17, 20, 33, 62, 97, 138, 195, 236, 239, 244, 263, and combinations thereof, wherein the amino acid position is relative to the amino acids sequence of rat arylsulfotransferase IV SEQ ID NO: 1, and (2) an amino acid sequence having at least 60% sequence identity with amino acids sequence SEQ ID NO: 1, and

(ii) a sulfo group donor,

in conditions suitable for a transfer of the sulfo group from the sulfo group donor to PAP to obtain PAPS.

The methods may further comprise a step of recovering the so-formed sulfated substrate.

A method disclosed herein may be for recycling PAP into PAPS, and may comprise at least a step of contacting said PAP with:

a) a non-naturally occurring mutated aryl sulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, and (ii) an amino acid sequence having at least 60% sequence identity with amino acids sequence SEQ ID NO: 1, and

b) a sulfo group donor in conditions suitable for a transfer of the sulfo group from the sulfo group donor to PAP to obtain PAPS.

In some embodiments, the sequences of the non-naturally occurring mutated arylsulfotransferases may have at least 75%, 80%, 85%, 90%, 95%, or 99% identity over the entire sequence of SEQ ID NO:1.

In some embodiments, the sequences of the non-naturally occurring mutated arylsulfotransferases may have at least 85%, 90%, 95%, or 99% identity over the entire sequence of SEQ ID NO:1.

In some embodiments, the sequences of the non-naturally occurring mutated arylsulfotransferases may have at least 90%, 95%, or 99% identity over the entire sequence of SEQ ID NO:1.

In some embodiments, the sequences of the non-naturally occurring mutated arylsulfotransferases may have at least 90% identity over the entire sequence of SEQ ID NO:1.

In some embodiments, the sequences of the non-naturally occurring mutated arylsulfotransferases may have at least 95% identity over the entire sequence of SEQ ID NO:1.

In some embodiments, the sequences of the non-naturally occurring mutated arylsulfotransferases may have at least 99% identity over the entire sequence of SEQ ID NO:1.

A method as disclosed herein may be for synthesizing a heparin.

When a method for sulfating a substrate comprise the conversion of PAP in PAPS with a mutated arylsulfotransferase as disclosed herein, the sulfotransferase used for sulfating the substrate may be different from the mutated arylsulfotransferase. Sulfation of a substrate may be carried out with an O-sulfotransferase (OST) enzyme, such as for example 2-OST, 3-OST, 3-OST-1, 3-OST-3, 6-OST, 6-OST-1, 6-OST3, or a N-sulfotransferase such as NDST1, NDST2.

Step of sulfation of a substrate and step of converting PAP in PAPS may be carried out sequentially or simultaneously in a one-pot reaction.

Step of converting PAP in PAPS may comprise providing a reaction mixture comprising PAP, an arylsulfotransferase as disclosed herein and a sulfo group donor.

In some embodiments, step of sulfation of a substrate of the methods disclosed herein may comprise a plurality of sub-steps a₁), a₂), a₃), . . . , during which the substrate may undergo successive enzymatically catalyzed reactions. Those reactions may be sulfation at different locations within the substrate, carried out by different sulfotransferases using PAPS as sulfo donor group. The different sulfotransferases may be, for example, different OSTs. Each sub-step a₁), a₂), a₃), . . . , in which a sulfation occur may be associated with a single step of converting PAP in PAPS or a plurality of associated sub-steps b₁), b₂), b₃), . . . , during which the PAP resulting from the different sulfation steps is converted in PAPS by a mutated arylsulfotransferase as disclosed herein.

In some embodiments, step of sulfation of a substrate may comprise a plurality of simultaneous or sequential sub-steps a₁), a₂), a₃), . . . , aₙ), and wherein at least two sub-steps comprise each a sulfation catalyzed by a sulfotransferase using PAPS as a sulfo group donor to obtain PAP and a sulfated substrate.

In some embodiments, step of converting PAP in PAPS may comprise a single step or a plurality of simultaneous or sequential sub-steps b₁), b₂), b₃), . . . , bₙ), and wherein the PAP obtained at each sub-steps a₁), a₂), a₃) during which a sulfation catalyzed by a sulfotransferase using PAPS has occurred is converted in PAPS.

An aspect of the disclosure is directed to a PAPS regeneration system usable in a method for sulfation of a polysaccharide substrate. A PAPS regeneration system may be used at step of converting PAP in PAPS. The method can be of a type wherein the sulfation of a polysaccharide substrate is catalyzed by a sulfotransferase, such as one or more OSTs, with a conversion of 3′-phosphoadenosine-5′-phosphosulfate (PAPS) to adenosine 3′,5′-diphosphate (PAP). The sulfation process can be coupled with the PAPS regeneration system allowing an enzymatic regeneration of the 3′-phosphoadenosine-5′-phosphosulfate from the adenosine 3′,5′-diphosphate. The enzymatic regeneration system employs a mutated non-naturally occurring arylsulfotransferase as disclosed herein and an aryl sulfate as a substrate.

In a PAPS regeneration system as disclosed herein, the mutated non-naturally occurring arylsulfotransferase can be grafted to a support. Suitable support may be a bead, a plate, a cellulose sheet. Therefore, reaction vessel may contain a reaction mixture comprising the substrate to be sulfated, one or more sulfotransferases distinct from the mutated arylsulfotransferase as disclosed herein, and PAPS as a sulfo donor group, and grafted to a support the mutated arylsulfotransferase allowing to continuously convert PAP into PAPS.

A mutated arylsulfotransferase as disclosed herein may be grafted, covalently or not, to a suitable support according to any known method in the art.

Coupling a sulfotransferase catalyzed sulfation reaction with a PAPS regeneration system as disclosed herein can provide a further advantage of generating PAPS utilized in the reaction directly from PAP. That is, the reaction mixture can be formulated to combine PAP with a PAPS regeneration system prior to or simultaneously with addition of a sulfotransferase to the reaction mixture. The mutated arylsulfotransferase can then generate PAPS from the PAP for use by the sulfotransferase, thereby alleviating the need of supplying any of the more expensive and unstable PAPS to the reaction mixture.

Time and Temperature

The mutated arylsulfotransferase as disclosed herein may be contacted with PAP and a sulfo donor group for a time period sufficient to catalyze the production of PAPS from the PAP by the arylsulfotransferase as disclosed herein utilizing the sulfo group donor as a substrate, such as for example a time period from about 1 minute to about 90 minutes, from about 10 minutes to about 30 minutes.

In some embodiments, the mutated arylsulfotransferase as disclosed herein may be contacted with PAP and a sulfo donor group for a time period compatible with the production of PAPS from the PAP by the arylsulfotransferase in industrial scaled-up process, such as about 2 hrs, or about 3 hrs, or about 6 hrs, or about 12 hrs, or about 24 hrs, or about 48 hrs, or about 72 hrs.

A temperature of reaction may from about 20° C. to about 40° C., or from about 25° C. to about 37° C., or from about 30° C. to about 35° C. For example, a temperature of reaction may be at about 37° C. As other example, a temperature of reaction may be at about 40° C.

Sulfo Group Donor

The sulfo group donor may be an aryl sulfate compound. An aryl sulfate compound may be p-Nitrophenyl sulfate (pNPS).

Substrates

In a method for sulfating a substrate where a mutated arylsulfotransferase as disclosed herein is used to convert PAP in PAPS, a substrate to be sulfated may be selected in a group comprising a polysaccharide, an heparan, an heparosan sulfate, a chemically desulfated N-sulfated (CDSNS) heparin, a glycosaminoglycan (GAG), an heparan sulfate or a sulfated heparin.

A polysaccharide substrate may be partially sulfated prior to reaction mixture incubation. In some embodiments, the sulfated polysaccharide is a glycosaminoglycan (GAG), such as for example a heparan sulfate (HS). In some embodiments, the sulfated polysaccharide is an HS that is an anticoagulant-active HS, an antithrombin-binding HS, a fibroblast growth factor (FGF)-binding HS, a herpes simplex virus envelope glycoprotein D-binding HS or has a combination of these properties.

In some embodiments, a substrate to be sulfated may undergo further to sulfation at least one additional enzymatically catalyzed reaction. This or these additional reaction(s) may be carried out before or after the sulfation.

A substrate to be sulfated may be a polysaccharide substrate previously N,O-desulfated and re-N-sulfated polysaccharide, such as for example a chemically desulfated N-sulfated (CDSNS) heparin. For example, a polysaccharide, such as CDSNS, can be reacted with a particular OST in presence of PAPS to produce a sulfated polysaccharide intermediate product that can then be reacted subsequently with a different OST in presence PAPS to further sulfate the polysaccharide at different locations. This sequential process of reacting the polysaccharide substrate with different OSTs can be continued until a final polysaccharide is produced exhibiting desired biological activities. The PAP resulting from each successive sulfation step may be then converted in PAPS in a single step or during successive steps for example succeeding to each sulfation step

The sulfation methods disclosed herein allows producing a multitude of sulfated polysaccharides, such as heparan sulfate molecules having varied biological activities by selecting appropriate sulfotransferases and by sequentially controlling the addition of those sulfotransferases to the reaction system to facilitate appropriate timing of sulfation of the polysaccharide. For example, heparan sulfate having specific biological activities which can be synthesized includes anticoagulant heparan sulfate, heparin, fibroblast growth factor-2-binding activity, herpes simplex virus glycoprotein D (gD)-binding HS, and fibroblast growth factor 2 (FGF2) receptor-binding HS. Only two or three enzymatic steps are required for the synthesis of each of these biologically-active heparan sulfate molecules. Thus, the methods disclosed herein, because of the high-yield PAPS regenerating system, provide efficient and effective methods for the large-scale synthesis of a wide range of heparan sulfate with specific activities.

In some embodiments, the sulfated polysaccharide substrate can be a glycosaminoglycan (GAG). GAGs are the most abundant heteropolysaccharides in the body. These molecules are long unbranched polysaccharides containing a repeating disaccharide unit. The disaccharide units can contain either of two modified sugars: N-acetylgalactosamine (GalNAc) or N-acetylglucosamine (GlcNAc) and a uronic acid such as glucuronate or iduronate. GAGs are highly negatively charged molecules, with extended conformation that imparts high viscosity to the solution. Along with the high viscosity of GAGs comes low compressibility, which makes these molecules ideal for a lubricating fluid in the joints. At the same time, their rigidity provides structural integrity to cells and provides passageways between cells, allowing for cell migration. The specific GAGs of physiological significance are hyaluronic acid, dermatan sulfate, chondroitin sulfate, heparin, heparan sulfate (including heparin), and keratan sulfate. Thus, in some embodiments, the sulfated polysaccharide product is a HS. In some embodiments, the sulfated polysaccharide product is an anticoagulant-active HS, an antithrombin-binding HS, an FGF-binding HS, and an HSV gD-binding HS.

Heparin Synthesis

In some embodiments, the presently disclosed subject matter provides a method of synthesizing a heparin compound.

Heparin has been known as a heparan sulfate, a highly acidic linear polysaccharide with a very variable structure, having an anticoagulant activity. Therefore, the presently disclosed subject matter provides a method of synthetizing heparin, low-molecular weight heparins such as low-molecular weight heparin having a weight average molecular weight of 4000 to 6000 and that has been increasingly used because of its less side effects such as bleeding, or an heparan sulfate having an anticoagulant activity; heparin, low-molecular weight heparins, heparan sulfate and heparan sulfate precursors being commonly designated as heparin compound or heparin.

A method for synthesizing heparin may comprise obtaining a sulfated heparin precursor by sulfating the heparin precursor with at least one sulfotransferase and PAPS, said method including at least one step of converting PAP into PAPS by contacting said PAP with a non-naturally occurring mutated arylsulfotransferase comprising (1) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, and (2) an amino acid sequence having at least 60% sequence identity with amino acids sequence SEQ ID NO: 1.

A method for synthesizing heparin may comprise sulfating a heparin precursor with a sulfotransferase and PAPS in conditions suitable to transfer a sulfo group from PAPS to the heparin precursor to be sulfated and to obtain a heparin precursor and PAP, converting the PAP so-obtained into PAPS by contacting the PAP with:

(i) a non-naturally occurring mutated arylsulfotransferase comprising (1) an amino acid substitution in at least one amino acid position selected in the group of positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, and (2) an amino acid sequence having at least 60% sequence identity with amino acids sequence SEQ ID NO: 1, and

(ii) a sulfo group donor.

in conditions suitable for a transfer of the sulfo group from the sulfo group donor to PAP to obtain PAPS.

Heparosan may be used as the polysaccharide raw material for the method for synthetizing heparin of the present invention. Heparosan is a polysaccharide composed of repetitive structures of a disaccharide composed of a glucuronic acid (GlcA) residue and an N-acetyl-D-glucosamine (GlcNAc) residue [→4)-β-D-GlcA-(1→4)-α-D-GlcNAc-(1→]. Heparosan can be produced, for example, by a fermentation method utilizing a bacterium having an ability to produce heparosan.

In some embodiments, the method disclosed herein for synthetizing heparin may use heparosan as heparin precursor. Heparin may be produced by subjecting heparosan as a starting material to different steps comprising N-deacetylation, N-sulfation, C5-epimerization, 2-O-sulfation, 6-O-sulfation, 3-O-sulfation. Heparin may be produced by subjecting heparosan to one, several or all of these steps or a combination of some of these steps or all of these steps. The method for producing heparin may further comprise a depolymerization step. The implementation order of the steps in the heparin production process is not particularly limited, so long as heparin having desired properties can be obtained.

In the method disclosed herein for synthetizing heparin these steps can be performed chemically or enzymatically or can be a combination between chemically and enzymatically performed steps. An enzymatic step may be, for example, a N-sulfation, a 2-O-sulfation enzymatic step, a 3-O-sulfation enzymatic step, a 6-O-sulfation enzymatic step or a succession or a combination of these steps. Such an enzymatic step can be performed by using, for example, a N-sulfotransferase enzyme such as NDST1 or NDST2, an O-sulfotransferase (OST) enzyme such as, for example, 2-OST, 3-OST, 3-OST-1, 3-OST-3, 6-OST, 6-OST-1, 6-OST-3. An enzymatic step for synthetizing heparin according to the method disclosed herein may be performed by more than one sulfotransferase or by a combination between one or more sulfotransferases and another enzyme, for example, a 2-OST and a C5 epimerase. The method as disclosed herein comprises at least one step with one sulfotransferase and PAPS including at least one step for converting PAP into PAPS by contacting said PAP with a non-naturally occurring arylsulfotransferase according to the present invention.

A method for synthesizing heparin may comprise further additional enzymatically catalyzed reactions.

A method for synthesizing heparin may comprise:
providing a saccharide substrate; elongating the saccharide substrate to a saccharide of a desired or predetermined length; performing an epimerization reaction; and performing one or more sulfation reactions with sulfotransferases and PAPS as sulfo donor group, whereby a heparin compound is synthesized, and
converting the PAP obtained at step a) into PAPS by contacting the PAP with a mutated non-naturally occurring arylsulfotransferase as disclosed herein and a sulfo group donor in conditions suitable for a transfer of the sulfo group from the sulfo group donor to PAP to obtain PAPS.

In some embodiments, the presently disclosed subject matter provides a method of synthesizing a heparin compound, comprising at least the steps consisting of:
providing a disaccharide substrate; elongating the disaccharide substrate to a tetrasaccharide; elongating the tetrasaccharide to a hexasaccharide or heptasaccharide, wherein the hexasaccharide or heptasaccharide comprises a N-sulfotransferase substrate residue; converting the N-sulfotransferase substrate residue on the hexasaccharide or heptasaccharide to a N-sulfo glucosamine (GlcNS) residue; performing an epimerization reaction; and performing one or more sulfation reactions selected from the group consisting of a N-sulfation, a 2-O-sulfation reaction, a 6-O-sulfation reaction, a 3-O-sulfation reaction in contact with at least 3′-phosphoadenosine 5′-phosphosulfate (PAPS) as a sulfo donor group, and combinations thereof, whereby a heparin compound and PAP are synthesized, and
converting the PAP obtained at step a) into PAPS by contacting the PAP with a mutated non-naturally occurring arylsulfotransferase as disclosed herein and a sulfo group donor in conditions suitable for a transfer of the sulfo group from the sulfo group donor to PAP to obtain PAPS.

Elongating the disaccharide substrate to a tetrasaccharide may be carried out using enzymes N-acetyl glucosaminyl transferase and heparosan synthase-2, and substrates glucuronic acid (GlcUA) and N-trifluoroacetyl glucosamine (GlcNTFA).

Elongating the tetrasaccharide to a heptasaccharide may be carried out using enzymes N-acetyl glucosaminyl transferase and heparosan synthase-2, and substrates glucuronic acid (GlcUA), N-trifluoroacetyl glucosamine (GlcNTFA), and N-acetylated glucosamine (GlcNAc).

Elongating a hexasaccharide to a heptasaccharide may be carried out using a glycosyl transferase. The glycosyl transferase may be a N-acetyl glucosaminyl transferase. In another aspect, the N-sulfotransferase substrate residue is a N-trifluoroacetyl glucosamine (GlcNTFA) residue.

Converting N-trifluoroacetyl glucosamine (GlcNTFA) residue(s) on the heptasaccharide to N-sulfo glucosamine (GlcNS) residues ay be carried out using N-sulfotransferase (NST), 3′-phosphoadenosine 5′-phosphosulfate (PAPS), triethylamine, CH₃OH, and H₂O.

Epimerizing the heptasaccharide may be carried out using C5-epimerase (C5-epi).

Sulfating the heptasaccharide may be carried out using 2-O-sulfotransferase (2-OST) and 3′-phosphoadenosine 5′-phosphosulfate (PAPS).

Sulfating the heptasaccharide may be carried out using 6-O-sulfotransferase (6-OST) and 3′-phosphoadenosine 5′-phosphosulfate (PAPS).

And sulfating the heptasaccharide may be carried out using 3-O-sulfotransferase (3-OST) and 3′-phosphoadenosine 5′-phosphosulfate (PAPS).

The invention will be further understood from the following non-limiting examples. The following examples are provided to describe in detail some of the representative, presently preferred methods and materials of the invention. These examples are provided for purposes of illustration of the inventive concepts and are not intended to limit the scope of the invention as defined by the appended claims.

### EXAMPLES

### Example 1: Preparation of Arylsulfotransferase Mutants

Mutants from rat's arylsulfotransferase IV (AST-IV-EC 2.8.2.9) were obtained by gene synthesis and cloned using the automated system BioXp™ 3200 from the company CODEX DNA according to manufacturer recommendations. into a pET-Duet vector.

The following AST IV mutants were prepared:

Single mutants:

Var01 (I17F)—SEQ ID NO:5,

Var04 (F20L)—SEQ ID NO:6,

Var03 (F20I)—SEQ ID NO:7,

Var05 (F138H)—SEQ ID NO:8,

Var06 (Y236F)—SEQ ID NO:9,

Var07 (M244N)—SEQ ID NO:10,

Var02 (I17Y)—SEQ ID NO:11,

Var08 (I239D)—SEQ ID NO:12,

Var09−01 (P6Q)—SEQ ID NO:14,

Var09−02 (P7D)—SEQ ID NO:15,

Var09−03 (L8A)—SEQ ID NO:16,

Var09−04 (V9G)—SEQ ID NO:17,

Var09−05 (V11L)—SEQ ID NO:18,

Var09−06 (W33R)—SEQ ID NO:19,

Var09−07 (K62D)—SEQ ID NO:20,

Var09−08 (A97S)—SEQ ID NO:21,

Var09−09 (N195D)—SEQ ID NO:22, and

Var09−10 (T263H)—SEQ ID NO:23.

Multiple Mutants:

Var09 comprising 10 combined mutations: P6Q-P7D-L8A-V9G-V11L-W33R-K62D-A97S-N195D-T263H—SEQ ID NO:13.

“Var09−P6Q” comprising 9 combined mutations: P7D-L8A-V9G-V11 L-W33R-K62D-A97S-N195D-T263H—SEQ ID NO:24. Mutation P6Q was removed.

“Var09−P7D” comprising 9 combined mutations: P6Q-L8A-V9G-V11L-W33R-K62D-A97S-N195D-T263H—SEQ ID NO:24. Mutation P7D was removed

“Var09−L8A” comprising 9 combined mutations: P6Q-P7D-V9G-V11L-W33R-K62D-A97S-N195D-T263H—SEQ ID NO: 26. Mutation L8A was removed.

“Var09−V9G” comprising 9 combined mutations: P6Q-P7D-L8A-V11L-W33R-K62D-A97S-N195D-T263H—SEQ ID NO: 27. Mutation V9G was removed.

“Var09−V11L” comprising 9 combined mutations: P6Q-P7D-L8A-V9G-W33R-K62D-A97S-N195D-T263H—SEQ ID NO: 28. Mutation V11L was removed.

“Var09−W33R” comprising 9 combined mutations: P6Q-P7D-L8A-V9G-V11L-A97S-N195D-T263H—SEQ ID NO: 29. Mutation W33R was removed.

“Var09−K62D” comprising 9 combined mutations: P6Q-P7D-L8A-V9G-V11L-W33R-K62D-A97S-N195D-T263H—SEQ ID NO: 30. Mutation K62D was removed.

“Var09−A97S” comprising 9 combined mutations: P6Q-P7D-L8A-V9G-V11 L-W33R-K62D-N195D-T263H—SEQ ID NO: 31. Mutation A97S was removed.

“Var09−N195D” comprising 9 combined mutations: P6Q-P7D-L8A-V9G-V11L-W33R-K62D-A97S-T263H—SEQ ID NO: 32. Mutation N195D was removed.

“Var09−T263H” comprising 9 combined mutations: P6Q-P7D-L8A-V9G-V11L-W33R-K62D-A97S-N195D—SEQ ID NO: 33. Mutation T263H was removed.

“Var09−K62D-T263H” comprising 8 combined mutations: P6Q-P7D-L8A-V9G-V11L-W33R-A97S-N195D—SEQ ID NO: 34. Mutations K62D and T263H were removed.

“Var09−K62D-N195D-T263H” comprising 7 combined mutations: P6Q-P7D-L8A-V9G-V11L-W33R-A97S—SEQ ID NO: 35. Mutations K62D, N195D and T263H were removed.

“Var09+I17F” comprising 11 combined mutations: P6Q-P7D-L8A-V9G-V11L-I17F-W33R-K62D-A97S-N195D-T263H—SEQ ID NO: 36.

“Var09+I17Y” comprising 11 combined mutations: P6Q-P7D-L8A-V9G-V11L-I17Y-W33R-K62D-A97S-N195D-T263H—SEQ ID NO: 37.

“Var09+F20I” comprising 11 combined mutations: P6Q-P7D-L8A-V9G-V11L-F20I-W33R-K62D-A97S-N195D-T263H—SEQ ID NO: 38.

“Var09+F20L” comprising 11 combined mutations: P6Q-P7D-L8A-V9G-V11L-F20L-W33R-K62D-A97S-N195D-T263H—SEQ ID NO: 39.

“Var09+F138H” comprising 11 combined mutations: P6Q-P7D-L8A-V9G-V11L-W33R-K62D-A97S-F138H-N195D-T263H—SEQ ID NO: 40.

“Var09+Y236F” comprising 11 combined mutations: P6Q-P7D-L8A-V9G-V11L-W33R-K62D-A97S-Y236F-N195D-T263H—SEQ ID NO: 41.

“Var09+I239D” comprising 11 combined mutations: P6Q-P7D-L8A-V9G-V11L-W33R-K62D-A97S-I239D-N195D-T263H—SEQ ID NO: 42.

“Var09+M244N” comprising 11 combined mutations: P6Q-P7D-L8A-V9G-V11L-W33R-K62D-A97S-N195D-M244N-T263H—SEQ ID NO: 43.

Var5A comprising 5 combined mutations: P6Q-P7D-L8A-V9G-V11L—SEQ ID NO: 44.

Var5B comprising 5 combined mutations: W33R-K62D-A97S-N195D-T263H—SEQ ID NO: 45.

“Var5A+W33R” comprising 6 combined mutations: P6Q-P7D-L8A-V9G-V11L-W33R—SEQ ID NO: 46.

“Var5A+K62D” comprising 6 combined mutations: P6Q-P7D-L8A-V9G-V11L-K62D—SEQ ID NO: 47.

“Var5A+A97S” comprising 6 combined mutations: P6Q-P7D-L8A-V9G-V11L-A97S—SEQ ID NO: 48.

“Var5A+N195D” comprising 6 combined mutations: P6Q-P7D-L8A-V9G-V11L-N195D—SEQ ID NO: 49.

“Var5A+T263H” comprising 6 combined mutations: P6Q-P7D-L8A-V9G-V11L-T263H—SEQ ID NO: 50.

“Var5B+P6Q” comprising 6 combined mutations: P6Q-W33R-K62D-A97S-N195D-T263H—SEQ ID NO: 51.

“Var5B+P7D” comprising 6 combined mutations: P7D-W33R-K62D-A97S-N195D-T263H—SEQ ID NO: 52.

“Var5B+L8A” comprising 6 combined mutations: L8A-W33R-K62D-A97S-N195D-T263H—SEQ ID NO: 53.

“Var5B+V9G” comprising 6 combined mutations: V9G-W33R-K62D-A97S-N195D-T263H—SEQ ID NO: 54.

“Var5B+V11L” comprising 6 combined mutations: V11L-W33R-K62D-A97S-N195D-T263H—SEQ ID NO: 55.

E. coli BL21 DE3 cells were transformed with the obtained plasmids encoding for the different mutants and for the wild-type AST IV (SEQ ID NO:1). 2 μL of cloned plasmids (1/10 diluted) were mixed with 40 volumes of BL21 electrocompetent cells which were then submitted to electroporation. In brief, 40 μl of cells were mixed with 2 μl of DNA, and transferred in the electroporation cuvette. Electroporation device was Gene pulser XCell electroporation system by BioRad used according to manufacturer recommendation.

The transformed cells were resuspended with 950 μL of SOC media (ThermoFisher Scientific). 200 μL of the resuspension were plated onto appropriate antibiotic (Ampicillin 100 mg/L LB plates) agar plate(s).

To produce wild-type enzyme and the different mutants of AST-IV, the transformed E. coli BL21 DE3 cells were grown in TB medium (Terrific Broth medium—ThermoFisher Scientific (0002123806), 37° C. until OD_{600 nm} reaches 0.5 measured with a Genesys 10 Bio by Thermo Scientific, then the production of mutants was induced by addition of 1 mM ITPG to the growth medium and maintaining the bacteria culture at 25° C. for 24 hours.

Cells expressing the wild-type and mutant enzymes were then harvested, washed with phosphate buffer and frozen at −80° C. until the analysis of the enzyme activity.

### Example 2: Analysis of Arylsulfotransferase Mutants Catalytic Activity

Materials and Methods

For the test of arylsulfotransferase activity, a colorimetric method has been developed in order to measure the amount of p-Nitrophenyl (pNP) released by the transfer of the sulfuryl group from p-Nitrophenyl sulfate (pNPS) to 3′,5′-adenosine-phosphate (PAP) for producing 3′-phosphoadenosine-5′-phosphosulfate (PAPS) according to the following scheme reaction:

PAP+pNPS→PAPS+pNP

For the test, 10 μL of E. coli BL21 DE3 cells expressing one mutant, as prepared in Example 1, at OD_{600 nm}=100 (corresponding to 30 ng/μL of enzyme) were incubated with (final concentrations):

pNPS 1 mM;

PAP 0.23 mM;

Phosphate buffer at pH 7.0; and

Glycerol 10%.

E. coli BL21 DE3 cells expressing the wild-type AST IV (EC 2.8.2.9) were used as control.

The reaction mixture was further incubated at 37° C., and the optical density (OD) was measured at 404 nm at 10, 30 or 90 minutes with a SpectraMax® 190 from Molecular Devices according to manufacturer's recommendations.

As negative control a sample of reaction mixture without PAP addition is added for each enzyme preparation.

The enzyme activity of the mutant is expressed as pNP production in arbitrary Unit of absorbance at 404 nm. Blank is subtracted to normalize the results.

Results

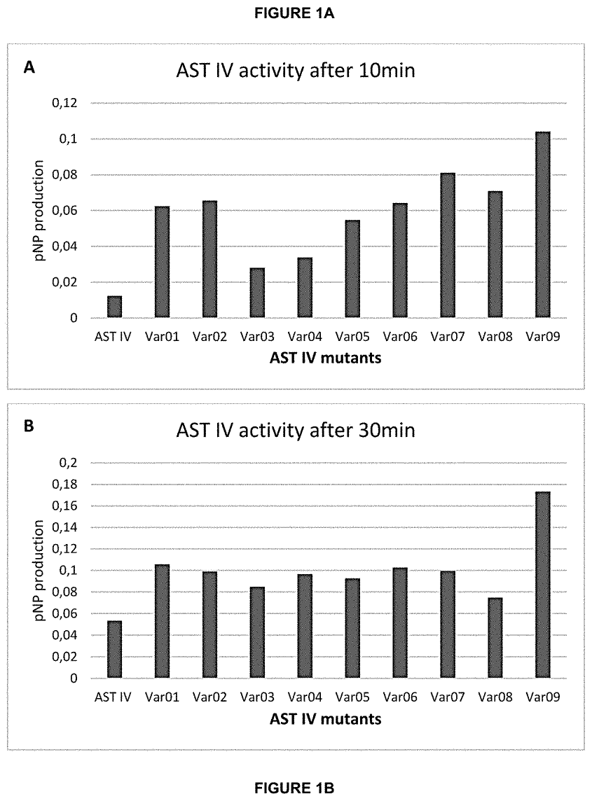

In a first series of experiments, the enzyme activity of the non-mutated (wild-type) arylsulfotransferase IV (AST IV) and of the different mutants Var01 to Var09 at 10 and 30 minutes. Results are presented on FIGS. 1A and 1B.

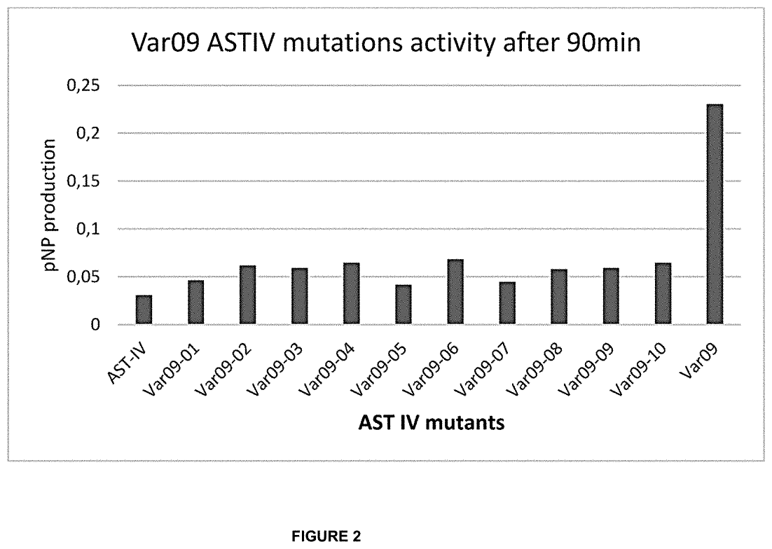

The enzyme activity of the non-mutated arylsulfotransferase IV (AST IV) and of the different mutants Var09−01 to Var09−10 and Var09 at 90 minutes are presented on FIG. 2.

As shown on the Figures, compared to the wild-type AST IV enzyme, the mutants have an increased enzyme activity of at least about 1.3-folds compared to the wild-type enzyme activity.

FIG. 1A shows that, 10 minutes after the initiation of the reaction, the single mutants Var01 to Var08 have an enzyme activity which is at least 2-folds the activity of the wild-type enzyme. Furthermore, the mutant Var05 has an enzyme activity increased by 4-folds and the mutants Var01, Var02, Var06, Var07 and Var08 have an enzyme activity increased by five-folds compared to the activity of the wild-type enzyme.

The multiple mutant Var09 has enzyme activity increased by at least 8-folds compared to the wild-type enzyme.

Those results show that the identified mutants have an increased catalytic efficiency compared to the wild-type enzyme.

FIG. 1A shows that, 30 minutes after the initiation of the reaction, the single mutants Var01 to Var08 have an enzyme activity which is increased by about 1.4 to about 1.9 times greater than the activity of the wild-type enzyme. The multiple mutant Var09 has an enzyme activity which is increased by at least about 3 times greater than the activity of the wild-type enzyme.

Those results show that the identified mutants have an increased catalytic rate compared to the wild-type enzyme.

FIG. 2 shows that, 90 minutes after the initiation of the reaction, the multiple mutant Var09 has an enzyme activity which is increased by at least about 7 times greater than the activity of the wild-type enzyme. The single mutants Var09−01 to Var09−10 have an enzyme activity which is increased from about 1.3 times to about 2.0-2.2 times greater than the activity of the wild-type enzyme. Notably, Var09−01 and Var09−07 have an enzyme activity increased by about 1.4 compared to the wild-type, and the Var09−02, Var09−03, Var09−04, Var09−06, Var09−08, Var09−09 and Var09−10 have an enzyme activity increased by about 1.8 to about 2.2-folds compared to the activity of the wild-type enzyme.

Those results show that the mutations of Var09, alone or in combination, induce an increase of the catalytic rate compared to the wild-type enzyme.

In a second series of experiments, the importance of the combined mutations of Var09 was explored with different constructs in which one, two or three substitutions from Var09 were removed.

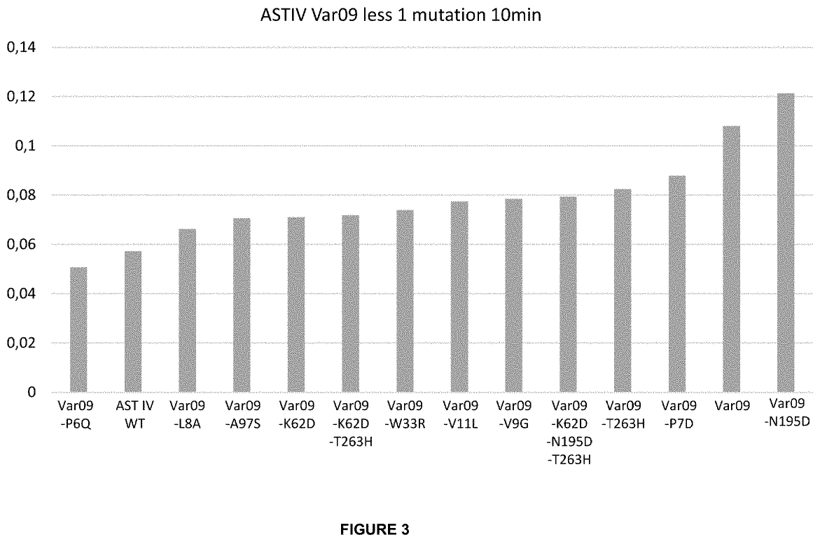

Catalytic activity of the mutants was measured as previously detailed above, 10 minutes after the initiation of the reaction. The results are presented on FIG. 3. As shown on FIG. 3, except for Var09−P6Q (mutant Var09 in which the substitution P6Q has been removed) and Var09−N195D, all the mutants maintained an activity above the wild-type enzyme but below Var09. The different substitutions appear to all contribute to a certain extent to the increase of the catalytic activity of Var09. Furthermore, they appear to cooperate together to enhance the catalytic activity.

The removal of substitution P60 reduced the catalytic activity of the mutant containing the other substitutions of Var09 below the activity of the wild-type enzyme (FIG. 3). When implemented alone the P60 substitution (Var09−01; FIG. 2) results in an increase of the catalytic activity, however in a modest manner. It appears therefore that P60 cooperates positively with the other mutations and could contribute more than the other mutations to strongly increase the catalytic activity of Var09 AST IV.

The removal of substitution N195D enhanced the catalytic activity of the mutant containing the other substitutions of Var09 above the activity of the mutant Var09 (FIG. 3). When implemented alone the N195D substitution (Var09−09; FIG. 2) results in a sensible increase of the catalytic activity compared to the wild-type enzyme. It appears therefore that, when combined with the other substitutions of Var09, N195D cooperates negatively with the other mutations resulting in a decrease of the catalytic activity of the mutated AST IV.

The removal of 2 (K62D & T263H) or 3 (K62D, N195D & T263H) substitutions results in mutants having an enhance catalytic activity compared to the wild-type AST IV enzyme, but lower than Var09. Interestingly, removal of N195D in the mutant already deprived of K62D and T263H does not result in a substantial increase of the enzyme activity which suggests that the negative cooperation of N195D with the other mutations as mentioned above does not apply for K62D & T263H combination. Of note, when implemented alone, each of the substitution K62D, N195D and T263H results in a sensible increase of the enzyme activity compared to the wild-type enzyme (see Var09−07, Var09−09 and Var09−10 on FIG. 2).

Those results taken together show that each of the substitution in Var09, while being able to increase the enzyme activity when taken alone, tends to cooperate with each other to further enhance the enzyme activity. In addition to Var09 and Var09−N195D, FIG. 3 results also show that several multiple mutants of AST display an enzyme activity above the wild-type enzyme activity (Var09−L8A, Var09−A97S, Var09−K62D, Var09−K62D-T263H, Var09−W33R, Var09−V11L, Var09−V9G, Var09−K62D-N195D-T263H, Var09−T263H).

In another set of experiments, each of the single substitution Var01 to Var08 was combined with Var09 to give “Var09+I17F”, “Var09+I17Y”, “Var09+F20I”, “Var09+F20L”, “Var09+F138H”, “Var09+Y236F”, “Var09+I239D” and “Var09+M244N”.

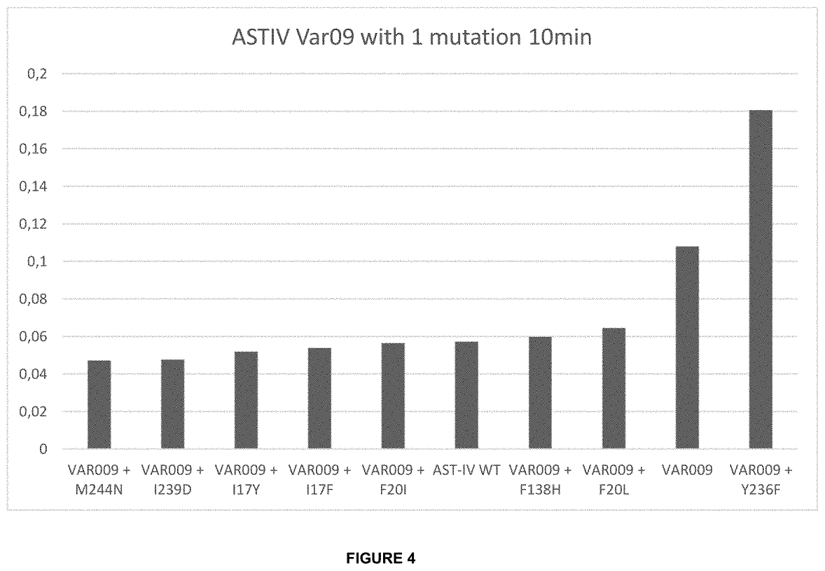

The results on the catalytic activity of the enzyme are presented on FIG. 4. Among the tested mutants, the combination of 10 substitutions of Var09 with Y236F resulted in strong activity increase far above the Var09 enzyme, suggesting that a mutation in this position cooperate positively with the other mutations to increase the enzyme activity.

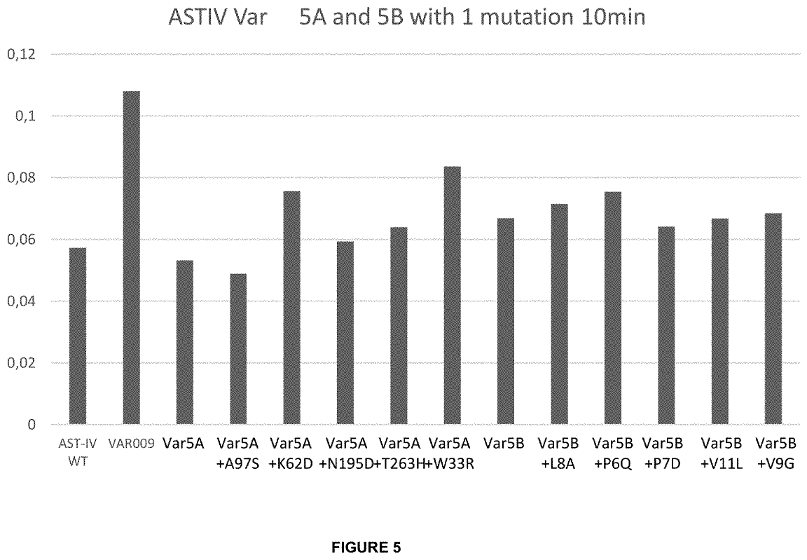

With respect to the conformation of the enzyme, the mutations in positions 6, 7, 8, 9 and 11 are randomly distributed to the 3-D conformation and can produce small rearrangements on the protein structure in order to promote a better activity, while the mutations in positions 33, 62, 97, 195 and 263 are rather at the surface of the 3-D conformation and can affect the thermal stability of the protein. It has therefore been explored the impact of these 2 classes of mutations with regard to their impact on the enzyme; by building two mutants: Var5B comprising the mutations at the surface (W33R, K62D, A97S, N195D, and T263H) and Var5A comprising the other mutations (P6Q, P7D, L8A, V9G, and V11L). Thereafter each new mutant was used to build a series of mutants in which one of each other mutation was added: Var5A+W33R, Var5A+K62D, Var5A+A97S, Var5A+N195D and Var5A+T263H on one hand, and Var5B+P6Q, Var5B+P7D, Var5B+L8A, Var5B+V9G, and Var5B+V11L. The catalytic activity of this new set of mutants was measured as previously indicated and compared to the wild-type enzyme and the Var09 mutant. Results are presented on FIG. 5.

The results shows that the combination of surface mutations increased the catalytic activity and that the addition of other mutations have slight positive or quite neutral effect. The combination of 5A mutations has a slight negative effect which can be rescued by addition of the other mutations in positions 33 or 62, 195, 263, with the mutations K62d and W33R having the strongest positive effect.

The arylsulfotransferase mutants disclosed herein have an increase enzyme rate and enzyme efficiency compared to the wild-type enzyme. Such mutants are therefore useful for converting, or recycling, PAP into PAPS. Those mutants may advantageously be used in enzymatic process requiring PAPS as a substrate, as for example the enzymatic production of sulfated polysaccharides (e.g., heparin), in order to efficiently convert, or recycle, PAP in PAPS and ensure maintaining a high rate and efficiency of such enzymatic process.

### Example 3: Analysis of Arylsulfotransferase Mutants Thermal Stability

Materials and Methods

For the test of arylsulfotransferase thermal stability, a Thermo Shift Assay was implemented using a C1000 Touch Thermal Cycler, Bio-Rad with a CFX96 Optical Reaction Module. Melting point of each variant is obtained with the software CFX Maestro from BioRad for the analysis of the results calculating the d(Fluorescence)/dT.

A temperature gradient from 15° C. during 15 seconds to 100° C. was applied with an increase of +0.5° C. every 5 seconds on different arylsulfotransferase mutants and wild-type enzyme.

The reaction mix used is as follows:

| | | |
|---|---|---|
| Enzyme | 20 | μg |
| Sodium Phosphate Buffer pH 7.0 | 50 | mM |

| | |
|---|---|
| Glycerol | 10% |
| Sypro Orange | 1x |

Results

In another set of experiments, the melting points of simple variants W33R, K62D, A97S, N195D, and T263H, constitutive of variant 5B, have been measured by a Thermo Shift Assay to determine their individual thermal stability effects. Results are presented on Table 4 below. Most of the variants show a low but significative increase in their respective melting point from 1° C. to 3° C. Variant Var5B displays a melting point increased by 6° C. compared to the wild-type enzyme which clearly benefits from the individual positive effects on thermal stability of these mutations.

The results are presented in the Table 4 below.

TABLE 4
Impact of amino acid substitution on the thermal
stability of the rat aryl sulfotransferase

| Variant | Mutation | Melting T° |
|---|---|---|
| AST-IV | wild-type | 50 |
| Var5B | W33R-K62D-A97S- | 56 |
| | N195D-T263H | |
| Var09-06 | W33R | 53 |
| Var09-07 | K62D | 53 |
| Var09-08 | A97S | 51 |
| Var09-09 | N195D | 52 |
| Var09-10 | T263H | 53 |

As shown in Table 4, the amino acid substitutions, alone or in combination, increase the thermal stability of the mutated enzyme from at least 1° C. up to 6° C. compared to the wild-type enzyme.

A more thermostable enzyme may be used at a higher temperature of incubation for enzyme reaction, for example for conversion of PAP into PAPS, which may accelerate the rate of reaction. This may be advantageously used in a bioprocess system, for example for sulfation of an heparosan sulfate, for example for heparin production, to enhance the rate of recycling PAP into PAPS. This may further enhance the yield of reaction and further reduce the costs of production.

### Example 4: Arylsulfotransferase Mutants Activities in a Coupling Reaction

Materials & Methods

A test for indirectly measuring arylsulfotransferase PAPS recycling activity in a sulfation coupling reaction with 20-sulfotransferase and C5-epimerase on N-Sulfated heparosan (NS heparosan) was implemented. Several arylsulfotransferase mutants were first purified before to be added in the reaction medium as below.

Enzymes Purification Method

AST-IV enzymes (wild-type and mutants), C5 epimerase (D-glucuronyl C5-epimerase; EC:5.1.3.17 from Danio rerio as referenced in Yi Qin at al., J Biol Chem. 2015 Feb. 20; 290(8):4620-4630. doi: 10.1074/jbc.M114.602201. Epub 2015 Jan. 7) and 2-OST (Heparan sulfate 2-O-sulfotransferase 1; EC:2.8.2.—from Cricetulus longicaudatus as referenced in M. Kobayashi at al. J Biol Chem. 1996 Mar. 29; 271(13):7645-53. doi: 10.1074/jbc.271.13.7645) were obtained by gene synthesis, cloned into pET-Duet vectors and produced in E. coli BL21 DE3 cells as detailed in example 1.

Enzymes were purified on a Ni-NTA resin. Ni-NTA was first equilibrated with a 50 mM Sodium Phosphate pH7, 20 mM Imidazole equilibration buffer. Enzyme lysates from bacteria were applied to the resin and incubated overnight on a rotating wheel at 4° C. Ni-NTA resin was washed 3 times with a 50 mM Sodium Phosphate pH7, 20 mM Imidazole washing buffer. 50 mM Sodium Phosphate pH7, 250 mM Imidazole Elution buffer was added for 2 hours on a rotating wheel at 4° C. Eluate was dialyzed with an Amicon Ultra (10 kDa cut-off) in a 50 mM Sodium Phosphate pH7, 10% glycerol buffer and stored at −80° C.

The arylsulfotransferase mutants tested were: “Var09” (SEQ ID NO: 13), “Var09−N195D” (SEQ ID NO: 32) and “Var09+Y236F” (SEQ ID NO: 41).

Enzymatic Reaction

The composition of enzymatic reaction medium is given in the Table 5 below:

TABLE 5
composition of enzymatic reaction medium

| | | |
|---|---|---|
| MES-KOH buffer pH 7 | 50 | mM |
| NaCl | 100 | mM |
| CaCl2•2H2O | 1.32 | mM |
| PNPS (4-Nitrophenylsulfate | 1-10 | mM |
| potassium Salt) | | |
| Reducing agent | 1 | mM |
| PAPS | 0.1-0.5 | mM |
| NSHeparosan | 1.2 | g/L |
| C5epimerase | 22 | mU/mL |
| 2OSulfotransferase | 60 | mU/mL |

| | |
|---|---|
| rat AST IV wild-type and | Amount according to |
| mutants | experiment (0.1 or 0.03 g/L) |

All the raw materials were firstly added and dissolved in the medium before addition of enzymes. The mix was then incubated at 37° C. for 24 hours under agitation. The enzymatic reaction was stopped with thermal shock at 95° C. during 45 min. Then, the sample was centrifuged at 9100 g at 4° C. for 10 min. Supernatant was recovered to be analyzed.

Sample Preparation for LC-MS Analysis

40 μL of sample resulting from enzymatic reaction (1 g/L) was mixed with 20 μL of citric acid (2 M) and 10 μL NaNO2 (1.05 M) and was incubated for 2 hrs at 65° C. under 1000 rpm agitation in Thermomixer. 30 μL of DNPH Dinitrophenyl hydrazine (51.5 mM) was added and incubated for 2 hrs at 65° C. under 1000 rpm agitation in Thermomixer. Samples were centrifugated and supernatants were transferred into HPLC vials.

LC-MS Analysis

TABLE 6
Ultra Performance Liquid Chromatography (UPLC)
analysis is applied with following parameters:

| | |
|---|---|
| Equipment | UPLC: Acquity Waters |

Settings

| Acquisition Software | Unifi | | |
|---|---|---|---|
| Column | SUMIPAX ODS Z-CLUE 250 × 2 mm 3 μm | | |
| Flow Rate | 0.3 mL/min | | |
| Run Time | 40 min | | |
| Column Temperature | 50° C. | | |
| Mobile Phase | Phase A: 50 mM HCOONH4 adjusted | | |
| | to pH 4.24 with HCOOH (5%) | | |
| | Phase B: Acetonitrile | | |
| Gradient | Time (min) | Phase A (%) | Phase B (%) |
| | 0 | 90 | 10 |
| | 13 | 80 | 20 |
| | 27 | 20 | 80 |
| | 27.1 | 90 | 10 |
| | 40 | 90 | 10 |

| | |
|---|---|
| UV | 365 nm |

After peak separation a mass spectrometry (MS) with a Xevo G2-XS QT of Waters is applied for peaks identification. MS is applied for the identification of the monosaccharide corresponding to each peak. The rate of sulfation is calculated as the percentage of the monosaccharides showing 2-O sulfation compared to the total of all monosaccharides analyzed.

Results

2-O sulfotransferase activity on N-Sulfated heparosan (NS heparosan) was measured in presence of C5-epimerase and several AST-IV mutants [Var-09 (SEQ ID NO: 13), Var-09-N195D (SEQ ID NO: 32) and Var-09+Y236F (SEQ ID NO: 41)]. Sulfation rates obtained are compared to wild-type AST-IV.

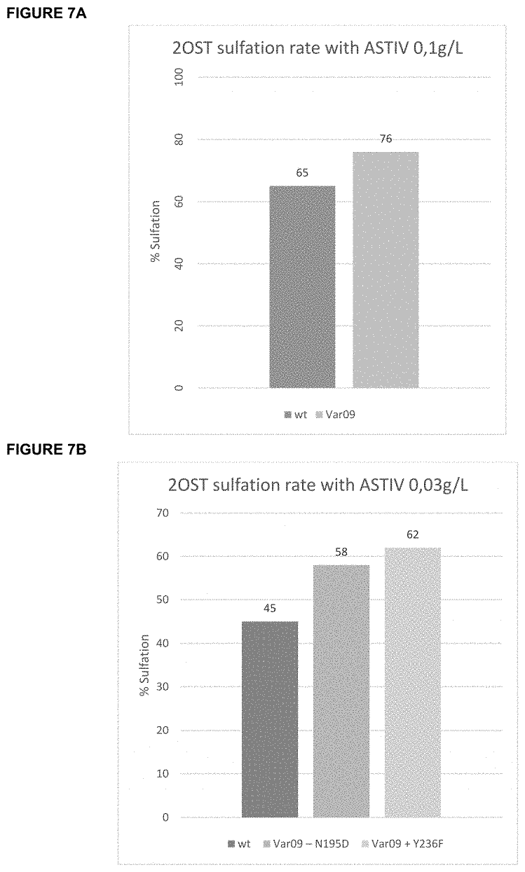

On two experiments using two different AST-IV enzyme quantities, respectively 0.1 g/L (FIG. 7A) and 0.03 g/L (FIG. 7B), sulfation rates obtained are clearly higher in presence of the three mutants Var-09, Var-09-N195D and Var-09+Y236F when compared to wild-type AST-IV.

In other words, the three mutants Var-09, Var-09-N195D and Var-09+Y236F allow increasing 2-O sulfation level compared to AST-IV WT, demonstrating an improvement in PAPS recycling activity compatible with their advantageous uses in a bioprocess system, for example for sulfation of an N-sulfated heparosan or heparan sulfate, for example for heparin production wherein an enhancement in recycling PAP to PAPS is needed.

### REFERENCES

Altschul S F. A protein alignment scoring system sensitive at all evolutionary distances. J Mol Evol. 1993; 36(3):290-300. doi:10.1007/BF00160485
Altschul S F, Gish W, Miller W, Myers E W, Lipman D J. Basic local alignment search tool. J Mol Biol. 1990; 215(3):403-410. doi:10.1016/S0022-2836(05)80360-2
Berger I, Guttman C, Amar D, Zarivach R, Aharoni A. The molecular basis for the broad substrate specificity of human sulfotransferase 1A1. PLoS One. 2011; 6(11):e26794. doi:10.1371/journal.pone.0026794
Burkart M D, Izumi M, Chapman E, Lin C H, Wong C H. Regeneration of PAPS for the enzymatic synthesis of sulfated oligosaccharides. J Org Chem. 2000; 65(18):5565-5574. doi:10.1021/jo000266o
Chen J, Avci F Y, Muñoz E M, et al. Enzymatic redesigning of biologically active heparan sulfate. J Biol Chem. 2005; 280(52):42817-42825. doi:10.1074/jbc.M504338200
Devereux J, Haeberli P, Smithies O. A comprehensive set of sequence analysis programs for the VAX. Nucleic Acids Res. 1984 Jan. 11; 12(1 Pt 1):387-95. doi: 10.1093/nar/12.1part1.387. PMID: 6546423; PMCID: PMC321012
Esko J D, Selleck S B. Order out of chaos: assembly of ligand binding sites in heparan sulfate. Annu Rev Biochem. 2002; 71:435-471. doi:10.1146/annurev.biochem.71.110601.135458
Fu L, Suflita M, Linhardt R J. Bioengineered heparins and heparan sulfates. Adv Drug Deliv Rev. 2016; 97:237-249. doi:10.1016/j.addr.2015.11.002
Guo W X, Yang Y S, Chen X, McPhie P, Jakoby W B. Changes in substrate specificity of the recombinant form of phenol sulfotransferase IV (tyrosine-ester sulfotransferase). Chem Biol Interact. 1994; 92(1-3):25-31. doi:10.1016/0009-2797(94)90050-7
Henikoff S, Henikoff J G. Amino acid substitution matrices from protein blocks. Proc Natl Acad Sci USA. 1992; 89(22):10915-10919. doi:10.1073/pnas.89.22.10915
Karlin S, Altschul S F. Applications and statistics for multiple high-scoring segments in molecular sequences. Proc Natl Acad Sci USA. 1993; 90(12):5873-5877. doi:10.1073/pnas.90.12.5873
Lin C H, Lin E S, Su T M, Hung K S, Yang Y S. A nano switch mechanism for the redox-responsive sulfotransferase. Biochem Pharmacol. 2012; 84(2):224-231. doi:10.1016/j.bcp.2012.04.003
Liu J, Thorp S C. Cell surface heparan sulfate and its roles in assisting viral infections. Med Res Rev. 2002; 22(1):1-25. doi:10.1002/med.1026
Marshall A D, Darbyshire J F, Hunter A P, McPhie P, Jakoby W B. Control of activity through oxidative modification at the conserved residue Cys66 of aryl sulfotransferase IV. J Biol Chem. 1997; 272(14):9153-9160. doi:10.1074/jbc.272.14.9153
Paul P, Suwan J, Liu J, Dordick J S, Linhardt R J. Recent advances in sulfotransferase enzyme activity assays. Anal Bioanal Chem. 2012; 403(6):1491-1500. doi:10.1007/s00216-012-5944-4
Sheng J J, Saxena A, Duffel M W. Influence of phenylalanines 77 and 138 on the stereospecificity of aryl sulfotransferase IV. Drug Metab Dispos. 2004; 32(5):559-565. doi:10.1124/dmd.32.5.559
WO 2010/040973—Biosynthetic Heparin
Xiong J, Bhaskar U, Li G, et al. Immobilized enzymes to convert N-sulfo, N-acetyl heparosan to a critical intermediate in the production of bioengineered heparin. J Biotechnol. 2013; 167(3):241-247. doi:10.1016/j.jbiotec.2013.06.018
Zhou Z, Li Q, Xu R, Wang B, Du G, Kang Z. Secretory expression of the rat aryl sulfotransferases IV with improved catalytic efficiency by molecular engineering. 3 Biotech. 2019; 9(6):246. doi:10.1007/s13205-019-1781-x
Yi Qin, Jiyuan Ke, Xin Gu, Jianping Fang, Wucheng Wang, Qifei Cong, Jie U, Jinzhi Tan, Joseph S Brunzelle, Chenghai Zhang, Yi Jiang, Karsten Melcher, Jin-Ping Li, H Eric Xu, Kan Ding, Structural and functional study of D-glucuronyl C5-epimerase J Biol Chem. 2015 Feb. 20; 290(8):4620-4630. doi: 10.1074/jbc.M114.602201. Epub 2015 Jan. 7.
M Kobayashi, H Habuchi, O Habuchi, M Saito, K Kimata Purification and characterization of heparan sulfate 2-sulfotransferase from cultured Chinese hamster ovary cells J Biol Chem. 1996 Mar. 29; 271(13):7645-53. doi: 10.1074/jbc.271.13.7645

1. (canceled)

2. (canceled)

3. A method for sulfating a substrate, comprising at least a step of contacting said substrate to be sulfated with:
a) a non-naturally occurring mutated arylsulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, and (ii) an amino acid sequence having at least 60% sequence identity with amino acids sequence of SEQ ID NO: 1, with the proviso that when said arylsulfotransferase is the rat arylsulfotransferase IV, the mutations are not F138A and/or Y236A, and
b) a sulfo group donor in conditions suitable for a transfer of the sulfo group from the sulfo group donor to said substrate.

4. A method for sulfating a substrate, comprising at least a step of contacting said substrate to be sulfated with:
a) a non-naturally occurring mutated arylsulfotransferase comprising (i) an amino acid substitution in at least one amino acid position selected among positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, (ii) an amino acid sequence having at least 60% sequence identity with amino acids sequence of SEQ ID NO: 1 and (iii) having a sulfotransferase activity for converting adenosine 3′,5′-bisphosphate (PAP) into 3′-phosphoadenosine-5′-phosphosulfate (PAPS) at least 1.3 times the said activity of the rat arylsulfotransferase IV of SEQ ID NO: 1, and
b) a sulfo group donor in conditions suitable for a transfer of the sulfo group from the sulfo group donor to said substrate.

5. A method for sulfating a substrate with a sulfotransferase and PAPS in conditions suitable to transfer a sulfo group from PAPS to the substrate to be sulfated and to obtain a sulfated substrate and PAP or for recycling PAP into PAPS, comprising at least a step of converting the PAP so-obtained into PAPS by contacting the PAP with:
(i) a non-naturally occurring mutated arylsulfotransferase comprising (1) an amino acid substitution in at least one amino acid position selected from positions 6, 7, 8, 9, 11, 17, 20, 33, 62, 97, 138, 195, 236, 239, 244, 263, and combinations thereof, wherein the position is relative to the amino acids sequence of rat arylsulfotransferase IV SEQ ID NO: 1, and (2) an amino acid sequence having at least 60% sequence identity with amino acids sequence pf SEQ ID NO: 1, and
(ii) a sulfo group donor in conditions suitable for a transfer of the sulfo group from the sulfo group donor to PAP to obtain PAPS.

6. The method according to claim 5, wherein the non-naturally occurring mutated arylsulfotransferase has a sulfotransferase activity for converting adenosine 3′,5′-bisphosphate (PAP) into 3′-phosphoadenosine-5′-phosphosulfate (PAPS) at least 1.3 times the said activity of the rat arylsulfotransferase IV of SEQ ID NO: 1.

7. The method according to claim 3, wherein the substrate is sulfated with one or a plurality of sulfotransferases to carry out a plurality of sulfation.

8. The method according to claim 7, wherein the plurality of sulfation is carried out concomitantly or sequentially.

9. The method according to anyone claim 5, wherein the step of converting PAP into PAPS is carried out concomitantly with the sulfation or separately.

10. The method according to claim 9, wherein the step of sulfation and the step of converting PAP into PAPS are carried out concomitantly in a same reaction mixture.

11. The method according to claim 3, further comprising a step of recovering the so-sulfated substrate.

12. The method according to claim 3, wherein the substrate is selected from the group consisting of adenosine 3′,5′-bisphosphate (PAP), a polysaccharide, an heparan, an heparosan sulfate, a chemically desulfated N-sulfated (CDSNS) heparin, a glycosaminoglycan (GAG), an heparan sulfate or a sulfated heparin, and combinations thereof.

13. The method according to claim 3, wherein the method is for converting adenosine 3′,5′-bisphosphate (PAP) into 3′-phosphoadenosine-5′-phosphosulfate (PAPS).

14. The method according to claim 3, wherein the method is for preparing a heparin.

15. (canceled)

16. The method according to claim 3, wherein the sulfo group donor is an aryl sulfate compound.

17. The method according to claim 16, wherein the aryl sulfate compound is p-Nitrophenyl sulfate (pNPS).

18. The method according to claim 3, wherein the non-naturally occurring mutated arylsulfotransferase is grafted onto a support.

19. The method according to claim 3, wherein the non-naturally occurring mutated arylsulfotransferase has an amino acids sequence selected from SEQ ID NOs: 5 to 23, 25-35, 41, 45-47, and 49-56 or has n amino acids sequence having at least 60% identity with a sequence selected from SEQ ID NOs: 5 to 23, 25-35, 41, 45-47, and 49-56 and a sulfotransferase activity for converting adenosine 3′,5′-bisphosphate (PAP) into 3′-phosphoadenosine-5′-phosphosulfate (PAPS) at least about 1.3 times greater than the said activity of the rat arylsulfotransferase IV of SEQ ID NO: 1.

20. The method according to claim 4, wherein the non-naturally occurring mutated arylsulfotransferase has an amino acids sequence selected from SEQ ID NOs: 5 to 23, 25-35, 41, 45-47, and 49-56 or has an amino acids sequence having at least 60% identity with a sequence selected from SEQ ID NOs: 5 to 23, 25-35, 41, 45-47, and 49-56 and a sulfotransferase activity for converting adenosine 3′,5′-bisphosphate (PAP) into 3′-phosphoadenosine-5′-phosphosulfate (PAPS) at least about 1.3 times greater than the said activity of the rat arylsulfotransferase IV of SEQ ID NO: 1.

21. The method according to claim 5, wherein the non-naturally occurring mutated arylsulfotransferase has an amino acids sequence selected from SEQ ID NOs: 5 to 23, 25-35, 41, 45-47, and 49-56 or has an amino acids sequence having at least 60% identity with a sequence selected from SEQ ID NOs: 5 to 23, 25-35, 41, 45-47, and 49-56 and a sulfotransferase activity for converting adenosine 3′,5′-bisphosphate (PAP) into 3′-phosphoadenosine-5′-phosphosulfate (PAPS) at least about 1.3 times greater than the said activity of the rat arylsulfotransferase IV of SEQ ID NO: 1.

22. The method according to claim 4, wherein the sulfo group donor is an aryl sulfate compound.

23. The method according to claim 5, wherein the sulfo group donor is an aryl sulfate compound.
